(19) United States
(12) Patent Application Publication
(10) Pub. No.: US 2005/0260653 A1
(43) Pub. Date: Nov. 24, 2005

(54) Nucleic-acid programmable protein arrays

(76) Inventors: Joshua Labaer, Medfield, MA (US); Niroshan Ramachandran, Boston, MA (US)

Correspondence Address: FISH & RICHARDSON PC, P.O. BOX 1022, MINNEAPOLIS, MN, 55440-1022, US

(21) Appl. No.: 11/107,101

(22) Filed: Apr. 14, 2005

Related U.S. Application Data
(60) Provisional application No. 60/562,293, filed on Apr. 14, 2004.

(51) Int. Cl. C12Q 1/68; C12M 1/34; B05D 3/00

(52) U.S. Cl. 435/6; 435/287.2; 427/2.11

(57) ABSTRACT

Arrays of polypeptides can be generated by translation of nucleic acid sequences encoding the polypeptides at individual addresses on the array. This allows for the rapid and versatile development of a polypeptide microarray platform for analyzing and manipulating biological information. In one embodiment, one or more nucleic acids that include a coding region and an anchoring agent are to stably attached to the substrate. The substrate can also be modified to include a binding agent.

### CROSS-REFERENCE TO RELATED APPLICATIONS

This application claims priority to U.S. Application Serial No. 60/562,293, filed on Apr. 14, 2004, and incorporates its contents by reference in its entirety.

### STATEMENT OF GOVERNMENT SUPPORT

This project was funded by the United States NIH/NCI grant R21 CA99191-01. The United States government may have certain rights in the invention.

### BACKGROUND OF THE INVENTION

The concept of peptide and protein arrays has drawn considerable attention because this approach to high-throughput experimentation allows the direct analysis of discrete protein binding and enzymatic activities without the complications of adverse in vivo effects.

### SUMMARY OF THE INVENTION

The inventors have discovered, among other things, that arrays of polypeptides can be generated by translation of nucleic acid sequences encoding the polypeptides at individual addresses on the array. This allows for the rapid and versatile development of a polypeptide microarray platform for analyzing and manipulating biological information.

In one aspect, the invention features a method that includes: disposing, on a substrate, one or more nucleic acids that include a coding region and an anchoring agent, maintaining the substrate under conditions which enable the anchoring agent of each disposed nucleic acid to stably attached to the substrate, and contacting the substrate with a translation effector. The substrate can include a plurality of addresses. The nucleic acid and the anchoring agent can be disposed separately or concurrently (e.g., in a single solution).

Nucleic acid can be disposed at the different addresses, e.g., step-wise or in a multiplex format, e.g., using a plurality of pins or nozzles, e.g., to deliver nucleic acid separately to separate addresses.

In one embodiment, the nucleic acid is (covalently or non-covalently) bound to an anchoring agent that stably attaches the nucleic acid to the substrate.

The substrate can be planar, e.g., have a horizontal plane in which the addresses are located at different discrete locations. The surface of the substrate can be flat (e.g., a glass slide) or can include indentations (e.g., wells) or partitions (e.g. barriers) and so forth.

In one embodiment, the method includes amplifying, at each address, a first attached nucleic acid using a nucleic acid amplification technique. For example, the amplifying includes rolling circle amplification and concatamers are formed. In another example, the amplifying includes extension of a primer.

The nucleic acid can be, e.g., RNA or DNA. It may be linear or circular, e.g., supercoiled (positively or negatively supercoiled). The nucleic acids at the different addresses can have a common region that is invariant amount the nucleic acid of the different addresses (e.g., which may be a majority of all available addresses or some subset of the available addresses). The nucleic acid can also include a variant region, e.g., to allow for different amino acid sequences of interest to be include or to allow for other variations, e.g., random or controlled variations at one or more locations in a protein, e.g., in a domain such as a scaffold domain.

In one embodiment, the step of contacting the substrate with a translation effector includes disposing or flowing the translation effector onto the surface, for example, using a single dispensing action or multiple dispensing actions. In one embodiment, the substrate is also contacted with a transcription effector.

In one embodiment, the anchoring agent is covalently attached to the respective nucleic acid. In one example, the anchoring agent is incorporated into the nucleic acid, e.g., during synthesis of the nucleic acid. For example, the nucleic acid can be synthesized in the presence of a digoxygenin-nucleotide. In another example, the anchoring agent includes a crosslinking moiety that becomes covalently attached to the respective nucleic acid. In another example, the anchoring agent includes an intercalating agent, e.g., a psoralen moiety. The anchoring agent can include a capture component, e.g., a small organic molecule, e.g., biotin. The substrate can include a biotin-binding protein (e.g., avidin or streptavidin). The capture component can also be a peptide or protein. For example, it can include hexahistidine, and the substrate includes a metal, e.g., Ni2⁺. The capture component can be a peptide and the substrate includes a peptide binding agent (e.g., an antibody or a metal). In one embodiment, the capture component includes a thiol and the substrate includes a thiol reactive agent (or vice versa). In one embodiment, the anchoring agent includes a moiety that non-covalently interacts with nucleic acid. For example, the moiety is a nucleic acid binding protein, an intercalating agent, or a non-protein nucleic acid binding molecule.

In one embodiment, the anchoring agent includes a crosslinking moiety separated from a capture component (e.g., biotin) by a linker, e.g., a linker of between about 5-500, e.g., 5-50 Angstroms.

In one embodiment, the nucleic acid is stably attached to the substrate by a covalent bond.

In one embodiment, the coding region encodes a polypeptide that includes a first amino acid sequence, e.g., an amino acid sequence of interest, and an affinity tag. The affinity tag binds to a binding agent. The method can also include disposing the binding agent on the substrate. In some cases, it is useful to prepare a solution that includes the nucleic acid and the binding agent, and to dispose the solution onto the substrate.

The method can include forming aggregates, e.g., between molecules of the binding agent, and optional between molecules of the binding agent, and molecules of an agent that is a part of or becomes associated with the anchoring agent. Aggregates can be formed, e.g., by using a chemical crosslinker. The aggregates can include greater than 5, 8, or 10 protein molecules. The aggregates can be greater than 200 kDa, 500 kDa or 2000 kDa in molecular weight.

The method can include other features described herein.

In another aspect, the invention features a method that includes: disposing, on a planar substrate, one or more nucleic acids that include a coding region and an anchoring agent, and maintaining the substrate under conditions which enable the anchoring agent of each disposed nucleic acid to stably attached to the substrate.

In one embodiment, the nucleic acid is (covalently or non-covalently) bound to an anchoring agent that stably attaches the nucleic acid to the substrate.

In one embodiment, the method includes amplifying, at each address, a first attached nucleic acid using a nucleic acid amplification technique. For example, the amplifying includes rolling circle amplification and concatamers are formed. In another example, the amplifying includes extension of a primer.

The nucleic acid can be, e.g., RNA or DNA. It may be linear or circular, e.g., supercoiled (positively or negatively supercoiled).

In one embodiment, the step of contacting the substrate with a translation effector includes disposing or flowing the translation effector onto the surface, for example, using a single dispensing action or multiple dispensing actions. In one embodiment, the substrate is also contacted with a transcription effector.

In one embodiment, the anchoring agent is covalently attached to the respective nucleic acid. For example, the anchoring agent includes a crosslinking moiety that becomes covalently attached to the respective nucleic acid. In another example, the anchoring agent includes an intercalating agent, e.g., a psoralen moiety. The anchoring agent can include a capture component, e.g., a small organic molecule, e.g., biotin. For example, the substrate includes a biotin-binding protein (e.g., avidin or streptavidin). The capture component can also be a peptide or protein. For example, it can include hexahistidine, and the substrate includes a metal, e.g., Ni2⁺. The capture component can be a peptide and the substrate includes a peptide binding agent (e.g., an antibody or a metal). In one embodiment, the capture component includes a thiol and the substrate includes a thiol reactive agent (or vice versa). In one embodiment, the anchoring agent includes a moiety that non-covalently interacts with nucleic acid. For example, the moiety is a nucleic acid binding protein, an intercalating agent, or a non-protein nucleic acid binding molecule.

In one embodiment, the nucleic acid is stably attached to the substrate by a covalent bond.

The method can include other features described herein.

In another aspect, the invention features a method that includes: providing a substrate that includes a plurality of addresses, each addresses including a nucleic acid that includes a coding region and that is stably attached to the substrate, and contacting the substrate with a translation effector.

In one embodiment, the nucleic acid is (covalently or non-covalently) bound to an anchoring agent that stably attaches the nucleic acid to the substrate.

In one embodiment, the step of providing the substrate includes amplifying, at each address, a first attached nucleic acid using a nucleic acid amplification technique. For example, the amplifying includes rolling circle amplification and concatamers are formed. In another example, the amplifying includes extension of a primer.

The nucleic acid can be, e.g., RNA or DNA. It may be linear or circular, e.g., supercoiled (positively or negatively supercoiled).

In one embodiment, the step of contacting the substrate with a translation effector includes disposing or flowing the translation effector onto the surface, for example, using a single dispensing action or multiple dispensing actions. In one embodiment, the substrate is also contacted with a transcription effector.

In one embodiment, the anchoring agent is covalently attached to the respective nucleic acid. For example, the anchoring agent includes a crosslinking moiety that becomes covalently attached to the respective nucleic acid. In another example, the anchoring agent includes an intercalating agent, e.g., a psoralen moiety. The anchoring agent can include a capture component, e.g., a small organic molecule, e.g., biotin. For example, the substrate includes a biotin-binding protein (e.g., avidin or streptavidin). The capture component can also be a peptide or protein. For example, it can include hexahistidine, and the substrate includes a metal, e.g., Ni2⁺. The capture component can be a peptide and the substrate includes a peptide binding agent (e.g., an antibody or a metal). In one embodiment, the capture component includes a thiol and the substrate includes a thiol reactive agent (or vice versa). In one embodiment, the anchoring agent includes a moiety that non-covalently interacts with nucleic acid. For example, the moiety is a nucleic acid binding protein, an intercalating agent, or a non-protein nucleic acid binding molecule.

In one embodiment, the nucleic acid is stably attached to the substrate by a covalent bond.

The method can include other features described herein.

In another aspect, the invention features a method that includes: providing a substrate that includes an agent that can capture and stably attach a nucleic acid (e.g., a modified or unmodified nucleic acid) and an agent that can capture and stably attach an affinity tag. The substrate can be contacted with the nucleic acid to stably attach the nucleic acid to the substrate. For example, the nucleic acid can be modified to include a biotin or other small molecule agent (e.g., FK506 or digoxygenin) and the substrate can include a biotin binding protein or other moiety that specifically binds or reacts with the small molecule agent. The substrate can also include another protein that interacts with the affinity tag. Unmodified nucleic acids can be attached, e.g., using site-specific DNA binding proteins. In certain embodiments, the protein that interacts with the affinity tag and with the nucleic acid are the same.

The substrate can be contacted with a transcription and/or translation effector, to produce a protein encoded by the nucleic acid, the protein including the affinity tag. The substrate can include a plurality of addresses. The method can include other features described herein.

In another aspect, the invention features a method that includes: providing a plurality of coding nucleic acids, modifying each nucleic acid of the plurality to include an anchoring agent, and disposing each nucleic acid of the plurality at an address on a substrate. For example, each coding nucleic acid encodes a polypeptide that includes a first amino acid sequence and an affinity tag. Each address can further include a binding agent that recognizes the affinity tag. In one embodiment, each nucleic acid of the plurality is disposed at a different address. In one embodiment, some nucleic acids of the plurality are disposed at the same address. In another embodiment, some nucleic acids of the plurality are disposed at at least two different addresses.

In one embodiment, the step of providing at least one coding nucleic acid of the plurality includes extending a source nucleic acid using a polymerase and a tagged nucleotide. Exemplary tagged nucleotides can include a biotin or digoxygenin moiety The method can include other features described herein.

In another aspect, the invention features a method that includes: providing a plurality of coding nucleic acids, stably attaching each nucleic acid of the plurality at an address on a substrate, and translating each nucleic acid of the plurality with a translation. The stable attachment formed can be covalent or non-covalent.

In one embodiment, the substrate includes positively charged groups that can interact with negative charges on nucleic acid. In one embodiment, the nucleic acid is crosslinked to the substrate, e.g., at at least one position, or at a single position, or at fewer than three positions. For example, the position can be predetermined or specified, e.g., by using a modified nucleotide or a sequence that is recognized by the substrate (e.g., using a site-specific nucleic acid binding protein). In one embodiment, the nucleic acids of the plurality are stably attached by formation of a concatamer with a nucleic acid anchored to the surface. The method can include other features described herein.

In another aspect, the invention features a method that includes: providing a substrate that includes a plurality of addresses, each addresses including a nucleic acid that includes a coding region and an anchoring agent that stably attaches the nucleic acid to the substrate, and contacting the substrate with a translation effector. The method can include other features described herein.

In another aspect, the invention features a method that includes: providing a plurality of coding nucleic acids, each coding nucleic acid encodes a polypeptide that includes a first amino acid sequence and an affinity tag, and disposing a binding agent and each nucleic acid of the plurality at an address on a substrate, thereby forming an array including a plurality of addresses. In one embodiment, the nucleic acid and the binding agent are disposed on an outer layer of the substrate. For example the substrate includes a porous outer layer. The nucleic acid and/or binding agent can be disposed within the porous layer. In one embodiment, the nucleic acid and the binding agent are disposed on different layers. For example, the nucleic acid can be associated with an inner layer and the binding agent can be associated with an outer layer, or vice versa. It is also possible to have additional layers, e.g., between the layer associated with the nucleic acid and the layer associated with the binding agent. In one embodiment, the nucleic acid and the binding agent are disposed on the surface of the substrate.

In one embodiment, each address further includes a binding agent that recognizes the affinity tag.

In one embodiment, the binding agent and the nucleic acid are disposed as a single mixture.

In one embodiment, the method includes forming a plurality of mixtures, each mixture including at least one of the plurality of coding nucleic acids and the binding agent.

In one embodiment, the binding agent includes an anchoring agent and each coding nucleic acid includes an anchoring agent. For example, the nucleic acid includes an anchoring agent that includes biotin, and the mixture further includes a biotin binding protein and a crosslinker (e.g., an amine reactive compound). Exemplary binding agents include GST or an antibody. For example, the tag is GST and the binding agent is an antibody that specifically binds to GST.

In another aspect, the invention features a method that includes: contemporaneously providing (e.g., depositing) (i) a binding agent that can interact with a tag and (ii) a nucleic acid that can be stably attached to a substrate and that includes a sequence encoding a first amino acid sequence and the tag onto a substrate. For example, the step of depositing includes providing a mixture that includes the binding agent and the nucleic acid. The method can further include repeating the depositing for a plurality of nucleic acids, each being disposed at a different address on the substrate. The method can further include other features described herein.

In another aspect, the invention features a substrate that includes a plurality of addresses, wherein each address includes (i) a binding agent that can interact with a tag and (ii) a nucleic acid that can be stably attached to a substrate and that includes a nucleic acid sequence encoding a first amino acid sequence and the tag. The substrate can include other features described herein.

In another aspect, the invention features a substrate that includes (i) a binding agent that can interact with a tag and that is stably attached to the substrate, and (ii) a plurality of nucleic acids that are stably attached to the substrate and that includes a nucleic acid sequence encoding a first amino acid sequence and the tag, each nucleic acid of the plurality being located at a discrete location on the substrate. In one embodiment, the nucleic acids of the plurality are covalently attached to the substrate. In one embodiment, the binding agent is covalently attached to the substrate. In one embodiment, the nucleic acids of the plurality are covalently attached to an anchoring agent, which interacts with a protein stably attached to the substrate. In one embodiment, the nucleic acids of the plurality are covalently attached to a biotin-psoralen moiety, which interacts with a biotin-binding protein stably attached to the substrate.

In one embodiment, the nucleic acids of the plurality are supercoiled. The substrate can include other features described herein.

In another aspect, the invention features a substrate that comprises a plurality of layers and, optionally, a plurality of addresses. A nucleic acid encoding a polypeptide that includes a first sequence and an affinity tag is associated with at least one address of at least one of the layers. A binding agent that recognizes the affinity tag is associated with a corresponding address in the same or a different layer.

For example at least one of the layers can be porous (e.g., polyacrylamide or agarose). The nucleic acid and/or binding agent can be disposed within the porous layer. In one embodiment, the nucleic acid and the binding agent are associated with different layers. For example, the nucleic acid can be associated with an inner layer and the binding agent can be associated with an outer layer, or vice versa. It is also possible to have additional layers, e.g., between the layer associated with the nucleic acid and the layer associated with the binding agent.

In one aspect, the invention features an array including a substrate having a plurality of addresses. Each address of the plurality includes: (1) a nucleic acid (e.g., a DNA or an RNA) encoding a hybrid amino acid sequence which includes a test amino acid sequence and an affinity tag; and, optionally, (2) a binding agent that recognizes the affinity tag. Optionally, each address of the plurality also includes one or both of (i) an RNA polymerase; and (ii) a translation effector.

In a preferred embodiment, each test amino acid sequence in the plurality of addresses is unique. For example, a test amino acid sequence can differ from all other test amino acid sequence of the plurality by 1, or more amino acid differences, (e.g., about 2, 3, 4, 5, 8, 16, 32, 64 or more differences; and, by way of example, has about 800, 256, 128, 64, or 32, 16, 8, 4, or fewer differences). In another preferred embodiment, the test amino acid sequence encoded by the nucleic acid at each address of the plurality is identical to all other test amino acid sequences in the plurality of addresses. In a preferred embodiment, the affinity tag encoded by the nucleic acid at each address of the plurality is the same, or substantially identical to all other affinity tags in the plurality of addresses. In another preferred embodiment, the nucleic acid at each address of the plurality encodes more than one affinity tag. In yet another preferred embodiment, the affinity tag encoded by the nucleic acid at an address of the plurality differs from at least one other affinity tag in the plurality of addresses.

In a preferred embodiment, the affinity tag is fused directly to the test amino acid sequence, e.g., directly amino-terminal, or directly carboxy-terminal. In another preferred embodiment, the affinity tag is separated from the test amino acid by one or more linker amino acids, e.g., 1, 2, 3, 4, 5, 6, 8, 10, 12, 20, 30 or more amino acids, preferably about 1 to 20, or about 3 to 12 amino acids. The linker amino acids can include a cleavage site, flexible amino acids (e.g., glycine, alanine, or serine, preferably glycine), and/or polar amino acids. The linker and affinity tag can be amino-terminal or carboxy-terminal to the test amino acid sequence.

The nucleic acid can be a RNA, or a DNA (e.g., a single-stranded DNA, or a double stranded DNA). In a preferred embodiment, the nucleic acid includes a plasmid DNA or a fragment thereof; an amplification product (e.g., a product generated by RCA, PCR, NASBA); or a synthetic DNA.

The nucleic acid can further include one or more of: a transcription promoter; a transcription regulatory sequence; a untranslated leader sequence; a sequence encoding a cleavage site; a recombination site; a 3′ untranslated sequence; a transcriptional terminator; and an internal ribosome entry site. In one embodiment, the nucleic acid sequence includes a plurality of cistrons (also termed “open reading frames”), e.g., the sequence is dicistronic or polycistronic. In another embodiment, the nucleic acid also includes a sequence encoding a reporter protein, e.g., a protein whose abundance can be quantitated and can provide an indication of the quantity of test polypeptide fixed to the plate. The reporter protein can be attached to the test polypeptide, e.g., covalently attached, e.g., attached as a translational fusion. The reporter protein can be an enzyme, e.g., β-galactosidase, chloramphenicol acetyl transferase, β-glucuronidase, and so forth. The reporter protein can produce or modulate light, e.g., a fluorescent protein (e.g., green fluorescent protein, variants thereof, red fluorescent protein, variants thereof, and the like), and luciferase.

A circular plasmid can include a bacterial and/or phage origin of replication. A transcription start site (e.g., a T7 promoter), and a selectable marker such as an antibiotic resistance gene. Some exemplary plasmids include recombination sites for simple insertion of a sequence of interest, e.g., to excise a counter-selectable marker.

The transcription promoter can be a prokaryotic promoter, a eukaryotic promoter, or a viral promoter. In a preferred embodiment, the promoter is the T7 RNA polymerase promoter. The regulatory components, e.g., the transcription promoter, can vary among nucleic acids at different addresses of the plurality. For example, different promoters can be used to vary the amount of polypeptide produced at different addresses.

In one embodiment, the nucleic acid also includes at least one site for recombination, e.g., homologous recombination or site-specific recombination, e.g., a lambda att site or variant thereof; a lox site; or a FLP site. In a preferred embodiment, the recombination site lacks stop codons in the reading frame of a nucleic acid encoding a test amino acid sequence. In another preferred embodiment, the recombination site includes a stop codon in the reading frame of a nucleic acid encoding a test amino acid sequence.

In another embodiment, the nucleic acid includes a sequence encoding a cleavage site, e.g., a protease site, e.g., a site cleaved by a site-specific protease (e.g., a thrombin site, an enterokinase site, a PreScission site, a factor Xa site, or a TEV site), or a chemical cleavage site (e.g., a methionine, preferably a unique methionine (cleavage by cyanogen bromide) or a proline (cleavage by formic acid)).

The nucleic acid can include a sequence encoding a second polypeptide tag in addition to the affinity tag. The second tag can be C-terminal to the test amino acid sequence and the affinity tag can be N-terminal to the test amino acid sequence; the second tag can be N-terminal to the test amino acid sequence, and the affinity tag can be C-terminal to the test amino acid sequence; the second tag and the affinity tag can be adjacent to one another, or separated by a linker sequence, both being N-terminal or C-terminal to the test amino acid sequence. In one embodiment, the second tag is an additional affinity tag, e.g., the same or different from the first tag. In another embodiment, the second tag is a recognition tag. For example, the recognition tag can report the presence and/or amount of test polypeptide at an address. Preferably the recognition tag has a sequence other than the sequence of the affinity tag. In still another embodiment, a plurality of polypeptide tags (e.g., less than 3, 4, 5, about 10, or about 20 tags) are encoded in addition to the first affinity tag. Each polypeptide tag of the plurality can be the same as or different from the first affinity tag.

The nucleic acid sequence can further include an identifier sequence, e.g., a non-coding nucleic acid sequence, e.g., one that is synthetically inserted, and allows for uniquely identifying the nucleic acid sequence. The identifier sequence can be sufficient in length to uniquely identify each sequence in the plurality; e.g., it is about 5 to 500, 10 to 100, 10 to 50, or about 10 to 30 nucleotides in length. The identifier can be selected so that it is not complementary or identical to another identifier or any region of each nucleic acid sequence of the plurality on the array.

The test amino acid sequence can further include a protein splicing sequence or intein. The intein can be inserted in the middle of a test amino acid sequence. The intein can be a naturally-occurring intein or a mutated intein.

The nucleic acids encoding the test amino acid sequences can be obtained from a collection of full-length expressed genes (e.g., a repository of clones), a cDNA library, or a genomic library. The encoding nucleic acids can be nucleic acids (e.g., an mRNA or cDNA) expressed in a tissue, e.g., a normal or diseased tissue. The test polypeptides (i.e., test amino acid sequences) can be mutants or variants of a scaffold protein (e.g., an antibody, zinc-finger, polypeptide hormone etc.). In yet another embodiment, the test polypeptides are random amino acid sequences, patterned amino acids sequences, or designed amino acids sequences (e.g., sequence designed by manual, rational, or computer-aided approaches). The plurality of test amino acid sequences can include a plurality from a first source, and plurality from a second source. For example, the test amino acid sequences on half the addresses of an array are from a diseased tissue or a first species, whereas the sequences on the remaining half are from a normal tissue or a second species.

In a preferred embodiment, each address of the plurality further includes one or more second nucleic acids, e.g., a plurality of unique nucleic acids. Hence, the plurality in toto can encode a plurality of test sequences. For example, each address of the plurality can encode a pool of test polypeptide sequences, e.g., a subset of a library or clone bank. A second array can be provided in which each address of the plurality of the second array includes a single or subset of members of the pool present at an address of the first array. The first and the second array can be used consecutively.

In other preferred embodiments, each address of the plurality further includes a second nucleic acid encoding a second amino acid sequence.

In one preferred embodiment, each address of the plurality includes a first test amino acid sequence that is common to all addresses of the plurality, and a second test amino acid sequence that is unique among all the addresses of the plurality. For example, the second test amino acid sequences can be query sequences whereas the first amino test amino acid sequence can be a target sequence. In another preferred embodiment, each address of the plurality includes a first test amino acid sequence that is unique among all the addresses of the plurality, and a second test amino acid sequence that is common to all addresses of the plurality. For example, the first test amino acid sequences can be query sequences whereas the second amino test amino acid sequence can be a target sequence. The second nucleic acid encoding the second test amino acid sequence can include a sequence encoding a recognition tag and/or an affinity tag.

At at least one address of the plurality, the first and second amino acid sequences can be such that they interact with one another. In one preferred embodiment, they are capable of binding to each other. The second test amino acid sequence is optionally fused to a detectable amino acid sequence, e.g., an epitope tag, an enzyme, a fluorescent protein (e.g., GFP, BFP, variants thereof). The second test amino acid sequence can be itself detectable (e.g., an antibody is available which specifically recognizes it). In another preferred embodiment, one is capable of modifying the other (e.g., making or breaking a bond, preferably a covalent bond, of the other). For example, the first amino acid sequence is kinase capable of phosphorylating the second amino acid sequence; the first is a methylase capable of methylating the second; the first is a ubiquitin ligase capable of ubiquitinating the second; the first is a protease capable of cleaving the second; and so forth.

These embodiments can be used to identify an interaction or to identify a compound that modulates, e.g., inhibits or enhances, an interaction.

The binding agent can be attached to the substrate. For example, the substrate can be derivatized and the binding agent covalently attached thereto. The binding agent can be attached via a bridging moiety, e.g., a specific binding pair. (e.g., the substrate contains a first member of a specific binding pair, and the binding agent is linked to the second member of the binding pair, the second member being attached to the substrate).

In yet another embodiment, an insoluble substrate (e.g., a bead or particle), is disposed at each address of the plurality, and the binding agent is attached to the insoluble substrate. The insoluble substrate can further contain information encoding its identity, e.g., a reference to the address on which it is disposed. The insoluble substrate can be tagged using a chemical tag, or an electronic tag (e.g., a transponder). The insoluble substrate can be disposed such that it can be removed for later analysis.

Also featured is a database, e.g., in computer memory or a computer readable medium. Each record of the database can include a field for the amino acid sequence encoded by the nucleic acid sequence and a descriptor or reference for the physical location of the nucleic acid sequence on the array. Optionally, the record also includes a field representing a result (e.g., a qualitative or quantitative result) of detecting the polypeptide encoded by the nucleic acid sequence. The database can include a record for each address of the plurality present on the array. The records can be clustered or have a reference to other records (e.g., including hierarchical groupings) based on the result.

In another aspect, the invention features an array including a substrate having a plurality of addresses. Each address of the plurality includes: (1) an RNA encoding a hybrid amino acid sequence comprising a test amino acid sequence and an affinity tag; and (2) a binding agent that recognizes the affinity tag. Optionally, each address of the plurality also includes one or both of (i) a transcription effector; and (ii) a translation effector.

In a preferred embodiment, each test amino acid sequence in the plurality of addresses is unique. For example, a test amino acid sequence can differ from all other test amino acid sequence of the plurality by 1, or more amino acid differences, (e.g., about 2, 3, 4, 5, 8, 16, 32, 64 or more differences; and, by way of example, has about 800, 256, 128, 64, or 32, 16, 8, 4, or fewer differences). In another preferred embodiment, the test amino acid sequence encoded by the nucleic acid at each address of the plurality is identical to all other test amino acid sequences in the plurality of addresses. In a preferred embodiment, the affinity tag encoded by the nucleic acid at each address of the plurality is the same, or substantially identical to all other affinity tags in the plurality of addresses. In another preferred embodiment, the nucleic acid at each address of the plurality encodes more than one affinity tag. In yet another preferred embodiment, the affinity tag encoded by the nucleic acid at an address of the plurality differs from at least one other affinity tag in the plurality of addresses.

In a preferred embodiment, the affinity tag is fused directly to the test amino acid sequence, e.g., directly amino-terminal, or directly carboxy-terminal. In another preferred embodiment, the affinity tag is separated from the test amino acid by one or more linker amino acids, e.g., 1, 2, 3, 4, 5, 6, 8, 10, 12, 20, 30 or more amino acids, preferably about 1 to 20, or about 3 to 12 amino acids. The linker amino acids can include a cleavage site, flexible amino acids (e.g., glycine, alanine, or serine, preferably glycine), and/or polar amino acids. The linker and affinity tag can be amino-terminal or carboxy-terminal to the test amino acid sequence.

The nucleic acid can further include one or more of: a untranslated leader sequence; a sequence encoding a cleavage site; a recombination site; a 3′ untranslated sequence; and an internal ribosome entry site. In one embodiment, the nucleic acid sequence includes a plurality of cistrons (also termed “open reading frames”), e.g., the sequence is dicistronic or polycistronic. In another embodiment, the nucleic acid also includes a sequence encoding a reporter protein, e.g., a protein whose abundance can be quantitated and can provide an indication of the quantity of test polypeptide fixed to the plate. The reporter protein can be attached to the test polypeptide, e.g., covalently attached, e.g., attached as a translational fusion. The reporter protein can be an enzyme, e.g., β-galactosidase, chloramphenicol acetyl transferase, β-glucuronidase, and so forth. The reporter protein can produce or modulate light, e.g., a fluorescent protein (e.g., green fluorescent protein, variants thereof, red fluorescent protein, variants thereof, and the like), and luciferase.

In one embodiment, the nucleic acid also includes at least one site for recombination, e.g., homologous recombination or site-specific recombination, e.g., a lambda att site or variant thereof; a lox site; or a FLP site. In a preferred embodiment, the recombination site lacks stop codons in the reading frame of a nucleic acid encoding a test amino acid sequence. In another preferred embodiment, the recombination site includes a stop codon in the reading frame of a nucleic acid encoding a test amino acid sequence.

In another embodiment, the nucleic acid includes a sequence encoding a cleavage site, e.g., a protease site, e.g., a site cleaved by a site-specific protease (e.g., a thrombin site, an enterokinase site, a PreScission site, a factor Xa site, or a TEV site), or a chemical cleavage site (e.g., a methionine, preferably a unique methionine (cleavage by cyanogen bromide) or a proline (cleavage by formic acid)).

The nucleic acid can include a sequence encoding a second polypeptide tag in addition to the affinity tag. The second tag can be C-terminal to the test amino acid sequence and the affinity tag can be N-terminal to the test amino acid sequence; the second tag can be N-terminal to the test amino acid sequence, and the affinity tag can be C-terminal to the test amino acid sequence; the second tag and the affinity tag can be adjacent to one another, or separated by a linker sequence, both being N-terminal or C-terminal to the test amino acid sequence. In one embodiment, the second tag is an additional affinity tag, e.g., the same or different from the first tag. In another embodiment, the second tag is a recognition tag. For example, the recognition tag can report the presence and/or amount of test polypeptide at an address. Preferably the recognition tag has a sequence other than the sequence of the affinity tag. In still another embodiment, a plurality of polypeptide tags (e.g., less than 3, 4, 5, about 10, or about 20 tags) are encoded in addition to the first affinity tag. Each polypeptide tag of the plurality can be the same as or different from the first affinity tag.

The nucleic acid sequence can further include an identifier sequence, e.g., a non-coding nucleic acid sequence, e.g., one that is synthetically inserted, and allows for uniquely identifying the nucleic acid sequence. The identifier sequence can be sufficient in length to uniquely identify each sequence in the plurality; e.g., it is about 5 to 500, 10 to 100, 10 to 50, or about 10 to 30 nucleotides in length. The identifier can be selected so that it is not complementary or identical to another identifier or any region of each nucleic acid sequence of the plurality on the array.

The test amino acid sequence can further include a protein splicing sequence or intein. The intein can be inserted in the middle of a test amino acid sequence. The intein can be a naturally-occurring intein or a mutated intein.

The nucleic acids encoding the test amino acid sequences can be obtained from a collection of full-length expressed genes (e.g., a repository of clones), a cDNA library, or a genomic library. The encoding nucleic acids can be nucleic acids (e.g., an mRNA or cDNA) expressed in a tissue, e.g., a normal or diseased tissue. The test polypeptides (i.e., test amino acid sequences) can be mutants or variants of a scaffold protein (e.g., an antibody, zinc-finger, polypeptide hormone etc.). In yet another embodiment, the test polypeptides are random amino acid sequences, patterned amino acids sequences, or designed amino acids sequences (e.g., sequence designed by manual, rational, or computer-aided approaches). The plurality of test amino acid sequences can include a plurality from a first source, and plurality from a second source. For example, the test amino acid sequences on half the addresses of an array are from a diseased tissue or a first species, whereas the sequences on the remaining half are from a normal tissue or a second species.

In a preferred embodiment, each address of the plurality further includes one or more second nucleic acids, e.g., a plurality of unique nucleic acids. Hence, the plurality in toto can encode a plurality of test sequences. For example, each address of the plurality can encode a pool of test polypeptide sequences, e.g., a subset of a library or clone bank. A second array can be provided in which each address of the plurality of the second array includes a single or subset of members of the pool present at an address of the first array. The first and the second array can be used consecutively.

In other preferred embodiments, each address of the plurality further includes a second nucleic acid encoding a second amino acid sequence.

In one preferred embodiment, each address of the plurality includes a first test amino acid sequence that is common to all addresses of the plurality, and a second test amino acid sequence that is unique among all the addresses of the plurality. For example, the second test amino acid sequences can be query sequences whereas the first amino test amino acid sequence can be a target sequence. In another preferred embodiment, each address of the plurality includes a first test amino acid sequence that is unique among all the addresses of the plurality, and a second test amino acid sequence that is common to all addresses of the plurality. For example, the first test amino acid sequences can be query sequences whereas the second amino test amino acid sequence can be a target sequence. The second nucleic acid encoding the second test amino acid sequence can include a sequence encoding a recognition tag and/or an affinity tag.

At at least one address of the plurality, the first and second amino acid sequences can be such that they interact with one another. In one preferred embodiment, they are capable of binding to each other. The second test amino acid sequence is optionally fused to a detectable amino acid sequence, e.g., an epitope tag, an enzyme, a fluorescent protein (e.g., GFP, BFP, variants thereof). The second test amino acid sequence can be itself detectable (e.g., an antibody is available which specifically recognizes it). In another preferred embodiment, one is capable of modifying the other (e.g., making or breaking a bond, preferably a covalent bond, of the other). For example, the first amino acid sequence is kinase capable of phosphorylating the second amino acid sequence; the first is a methylase capable of methylating the second; the first is a ubiquitin ligase capable of ubiquitinating the second; the first is a protease capable of cleaving the second; and so forth.

These embodiments can be used to identify an interaction or to identify a compound that modulates, e.g., inhibits or enhances, an interaction.

The binding agent can be attached to the substrate. For example, the substrate can be derivatized and the binding agent covalent attached thereto. The binding agent can be attached via a bridging moiety, e.g., a specific binding pair. (e.g., the substrate contains a first member of a specific binding pair, and the binding agent is linked to the second member of the binding pair, the second member being attached to the substrate). In yet another embodiment, an insoluble substrate (e.g., a bead or particle), is disposed at each address of the plurality, and the binding agent is attached to the insoluble substrate. The insoluble substrate can further contain information encoding its identity, e.g., a reference to the address on which it is disposed. The insoluble substrate can be tagged using a chemical tag, or an electronic tag (e.g., a transponder). The insoluble substrate can be disposed such that it can be removed for later analysis.

In still another aspect, the invention features an array including a substrate having a plurality of addresses. Each address of the plurality includes: (1) a polypeptide comprising a test amino acid sequence and an affinity tag; and optionally (2) a binding agent. The binding agent is optimally capable of attaching to the affinity tag of the polypeptide. Optionally, each address of the plurality also includes a translation effector and/or a transcription effector.

In a preferred embodiment, each test amino acid sequence in the plurality of addresses is unique. For example, a test amino acid sequence can differ from all other test amino acid sequence of the plurality by 1, or more amino acid differences, (e.g., about 2, 3, 4, 5, 8, 16, 32, 64 or more differences; and, by way of example, has about 800, 256, 128, 64, or 32, 16, 8, 4, or fewer differences). In another preferred embodiment, the test amino acid sequence of the polypeptide is identical to all other test amino acid sequences in the plurality of addresses. In a preferred embodiment, the affinity tag of the polypeptide at each address of the plurality is the same, or substantially identical to all other affinity tags in the plurality of addresses.

In a preferred embodiment, the polypeptide has more than one affinity tag. In another embodiment, the polypeptide of an address has an affinity tag that differs from at least one other affinity tag of a polypeptide in the plurality of addresses.

In a preferred embodiment, the affinity tag is fused directly to the test amino acid sequence, e.g., directly amino-terminal, or directly carboxy-terminal. In another preferred embodiment, the affinity tag is separated from the test amino acid by one or more linker amino acids, e.g., 1, 2, 3, 4, 5, 6, 8, 10, 12, 20, 30 or more amino acids, preferably about 1 to 20, or about 3 to 12 amino acids. The linker amino acids can include a cleavage site, flexible amino acids (e.g., glycine, alanine, or serine, preferably glycine), and/or polar amino acids. The linker and affinity tag can be amino-terminal or carboxy-terminal to the test amino acid sequence.

In another embodiment, each address of the plurality further includes a nucleic acid. The nucleic acid at each address of the plurality encodes the polypeptide. The nucleic acid can be a RNA, or a DNA (e.g., a single-stranded DNA, or a double stranded DNA). In a preferred embodiment, the nucleic acid includes a plasmid DNA or a fragment thereof; an amplification product (e.g., a product generated by RCA, PCR, NASBA); or a synthetic DNA.

The nucleic acid can further include one or more of: a transcription promoter; a transcription regulatory sequence; a untranslated leader sequence; a sequence encoding a cleavage site; a recombination site; a 3′ untranslated sequence; a transcriptional terminator; and an internal ribosome entry site. In one embodiment, the nucleic acid sequence includes a plurality of cistrons (also termed “open reading frames”), e.g., the sequence is dicistronic or polycistronic.

The transcription promoter can be a prokaryotic promoter, a eukaryotic promoter, or a viral promoter. In a preferred embodiment, the promoter is the T7 RNA polymerase promoter. The regulatory components, e.g., the transcription promoter, can vary among nucleic acids at different addresses of the plurality. For example, different promoters can be used to vary the amount of polypeptide produced at different addresses.

In one embodiment, the nucleic acid also includes at least one site for recombination, e.g., homologous recombination or site-specific recombination, e.g., a lambda att site or variant thereof; a lox site; or a FLP site. In a preferred embodiment, the recombination site lacks stop codons in the reading frame of a nucleic acid encoding a test amino acid sequence. In another preferred embodiment, the recombination site includes a stop codon in the reading frame of a nucleic acid encoding a test amino acid sequence.

The nucleic acid sequence can further include an identifier sequence, e.g., a non-coding nucleic acid sequence, e.g., one that is synthetically inserted, and allows for uniquely identifying the nucleic acid sequence. The identifier sequence can be sufficient in length to uniquely identify each sequence in the plurality; e.g., it is about 5 to 500, 10 to 100, 10 to 50, or about 10 to 30 nucleotides in length. The identifier can be selected so that it is not complementary or identical to another identifier or any region of each nucleic acid sequence of the plurality on the array.

In another embodiment, the polypeptide further includes a reporter protein, e.g., a protein whose abundance can be quantitated and can provide an indication of the quantity of test polypeptide fixed to the plate. The reporter protein can be attached to the test polypeptide, e.g., covalently attached, e.g., attached as a translational fusion. The reporter protein can be an enzyme, e.g., β-galactosidase, chloramphenicol acetyl transferase, β-glucuronidase, and so forth. The reporter protein can produce or modulate light, e.g., a fluorescent protein (e.g., green fluorescent protein, variants thereof, red fluorescent protein, variants thereof, and the like), and luciferase.

In another embodiment, the polypeptide includes a cleavage site, e.g., a protease site, e.g., a site cleaved by a site-specific protease (e.g., a thrombin site, an enterokinase site, a PreScission site, a factor Xa site, or a TEV site), or a chemical cleavage site (e.g., a methionine, preferably a unique methionine (cleavage by cyanogen bromide) or a proline (cleavage by formic acid)).

The polypeptide can also include a sequence encoding a second polypeptide tag in addition to the affinity tag. The second tag can be C-terminal to the test amino acid sequence and the affinity tag can be N-terminal to the test amino acid sequence; the second tag can be N-terminal to the test amino acid sequence, and the affinity tag can be C-terminal to the test amino acid sequence; the second tag and the affinity tag can be adjacent to one another, or separated by a linker sequence, both being N-terminal or C-terminal to the test amino acid sequence. In one embodiment, the second tag is an additional affinity tag, e.g., the same or different from the first tag. In another embodiment, the second tag is a recognition tag. For example, the recognition tag can report the presence and/or amount of test polypeptide at an address. Preferably the recognition tag has a sequence other than the sequence of the affinity tag. In still another embodiment, a plurality of polypeptide tags (e.g., less than 3, 4, 5, about 10, or about 20 tags) are encoded in addition to the first affinity tag. Each polypeptide tag of the plurality can be the same as or different from the first affinity tag.

The test amino acid sequence can further includes a protein splicing sequence or intein. The intein can be inserted in the middle of a test amino acid sequence. The intein can be a naturally-occurring intein or a mutated intein.

A variety of test amino acid sequences can be disposed at different addresses of the plurality. For example, the test amino acid sequences can be polypeptides expressed in a tissue, e.g., a normal or diseased tissue. The test polypeptides can be mutants or variants of a scaffold protein (e.g., an antibody, zinc-finger, polypeptide hormone etc.). In yet another embodiment, the test polypeptides are random amino acid sequences, patterned amino acids sequences, or designed amino acids sequences (e.g., sequence designed by manual, rational, or computer-aided approaches). The plurality of test amino acid sequences can include a plurality from a first source, and plurality from a second source. For example, the test amino acid sequences on half the addresses of an array are from a diseased tissue or a first species, whereas the sequences on the remaining half are from a normal tissue or a second species.

In a preferred embodiment, each address of the plurality further includes one or more second polypeptides. Hence, the plurality, in toto, can encode a plurality of test polypeptides. For example, each address of the plurality can include a pool of test polypeptide sequences, e.g., a subset of polypeptides encoded by a library or clone bank. A second array can be provided in which each address of the plurality of the second array includes a single or subset of members of the pool present at an address of the first array. The first and the second array can be used consecutively.

In other preferred embodiments, each address of the plurality further includes a second polypeptide.

In one preferred embodiment, each address of the plurality includes a first test amino acid sequence that is common to all addresses of the plurality, and a second test amino acid sequence that is unique among all the addresses of the plurality. For example, the second test amino acid sequences can be query sequences whereas the first amino test amino acid sequence can be a target sequence. In another preferred embodiment, each address of the plurality includes a first test amino acid sequence that is unique among all the addresses of the plurality, and a second test amino acid sequence that is common to all addresses of the plurality. For example, the first test amino acid sequences can be query sequences whereas the second amino test amino acid sequence can be a target sequence. The second test amino acid sequence can include a recognition tag and/or an affinity tag.

At at least one address of the plurality, the first and second amino acid sequences can be such that they interact with one another. In one preferred embodiment, they are capable of binding to each other. The second test amino acid sequence is optionally fused to a detectable amino acid sequence, e.g., an epitope tag, an enzyme, a fluorescent protein (e.g., GFP, BFP, variants thereof). The second test amino acid sequence can be itself detectable (e.g., an antibody is available which specifically recognizes it). In another preferred embodiment, one is capable of modifying the other (e.g., making or breaking a bond, preferably a covalent bond, of the other). For example, the first amino acid sequence is kinase capable of phosphorylating the second amino acid sequence; the first is a methylase capable of methylating the second; the first is a ubiquitin ligase capable of ubiquitinating the second; the first is a protease capable of cleaving the second; and so forth. These embodiments can be used to identify an interaction or to identify a compound that modulates, e.g., inhibits or enhances, an interaction.

The binding agent can be attached to the substrate. For example, the substrate can be derivatized and the binding agent covalent attached thereto. The binding agent can be attached via a bridging moiety, e.g., a specific binding pair. (e.g., the substrate contains a first member of a specific binding pair, and the binding agent is linked to the second member of the binding pair, the second member being attached to the substrate). In yet another embodiment, an insoluble substrate (e.g., a bead or particle), is disposed at each address of the plurality, and the binding agent is attached to the insoluble substrate. The insoluble substrate can further contain information encoding its identity, e.g., a reference to the address on which it is disposed. The insoluble substrate can be tagged using a chemical tag, or an electronic tag (e.g., a transponder). The insoluble substrate can be disposed such that it can be removed for later analysis.

Also featured is a database, e.g., in computer memory or a computer readable medium. Each record of the database can include a field for the amino acid sequence of the polypeptide at an address and a descriptor or reference for the physical location of the address on the array. Optionally, the record also includes a field representing a result (e.g., a qualitative or quantitative result) of detecting the polypeptide. The database can include a record for each address of the plurality present on the array. The records can be clustered or have a reference to other records (e.g., including hierarchical groupings) based on the result.

The invention also features a method of providing an array. The method includes: (1) providing a substrate with a plurality of addresses; and (2) providing at each address of the plurality at least (i) a nucleic acid encoding an amino acid sequence comprising a test amino acid sequence and an affinity tag, and optionally (ii) a binding agent that recognizes the affinity tag.

The method can further include contacting each address of the plurality with one or more of (i) a transcription effector, and (ii) a translation effector. Optionally, the substrate is maintained under conditions permissive for the amino acid sequence to bind the binding agent. One or more addresses can then be washed, e.g., to remove at least one of (i) the nucleic acid, (ii) the transcription effector, (iii) the translation effector, and/or (iv) an unwanted polypeptide, e.g., an unbound polypeptide or unfolded polypeptide. The array can optionally be contacted with a compound, e.g., a chaperone; a protease; a protein-modifying enzyme; a small molecule, e.g., a small organic compound (e.g., of molecular weight less than 5000, 3000, 1000, 700, 500, or 300 Daltons); nucleic acids; or other complex macromolecules e.g., complex sugars, lipids, or matrix molecules.

The array can be further processed, e.g., prepared for storage. It can be enclosed in a package, e.g., an air- or water-resistant package. The array can be desiccated, frozen, or contacted with a storage agent (e.g., a cryoprotectant, an anti-bacterial, an anti-fungal). For example, an array can be rapidly frozen after being optionally contacted with a cryoprotectant. This step can be done at any point in the process (e.g., before or after contacting the array with an RNA polymerase; before or after contacting the array with a translation effector; or before or after washing the array). The packaged product can be supplied to a user with or without additional contents, e.g., a transcription effector, a translation effector, a vector nucleic acid, an antibody, and so forth.

In a preferred embodiment, each test amino acid sequence in the plurality of addresses is unique. For example, a test amino acid sequence can differ from all other test amino acid sequence of the plurality by 1, or more amino acid differences, (e.g., about 2, 3, 4, 5, 8, 16, 32, 64 or more differences; and, by way of example, has about 800, 256, 128, 64, or 32, 16, 8, 4, or fewer differences). In another preferred embodiment, the test amino acid sequence encoded by the nucleic acid at each address of the plurality is identical to all other test amino acid sequences in the plurality of addresses. In a preferred embodiment, the affinity tag encoded by the nucleic acid at each address of the plurality is the same, or substantially identical to all other affinity tags in the plurality of addresses. In another preferred embodiment, the nucleic acid at each address of the plurality encodes more than one affinity tag. In yet another preferred embodiment, the affinity tag encoded by the nucleic acid at an address of the plurality differs from at least one other affinity tag in the plurality of addresses.

In a preferred embodiment, the affinity tag is fused directly to the test amino acid sequence, e.g., directly amino-terminal, or directly carboxy-terminal. In another preferred embodiment, the affinity tag is separated from the test amino acid by one or more linker amino acids, e.g., 1, 2, 3, 4, 5, 6, 8, 10, 12, 20, 30 or more amino acids, preferably about 1 to 20, or about 3 to 12 amino acids. The linker amino acids can include a cleavage site, flexible amino acids (e.g., glycine, alanine, or serine, preferably glycine), and/or polar amino acids. The linker and affinity tag can be amino-terminal or carboxy-terminal to the test amino acid sequence.

The nucleic acid can be a RNA, or a DNA (e.g., a single-stranded DNA, or a double stranded DNA). In a preferred embodiment, the nucleic acid includes a plasmid DNA or a fragment thereof; an amplification product (e.g., a product generated by RCA, PCR, NASBA); or a synthetic DNA.

The nucleic acid can further include one or more of: a transcription promoter; a transcription regulatory sequence; a untranslated leader sequence; a sequence encoding a cleavage site; a recombination site; a 3′ untranslated sequence; a transcriptional terminator; and an internal ribosome entry site. In one embodiment, the nucleic acid sequence includes a plurality of cistrons (also termed “open reading frames”), e.g., the sequence is dicistronic or polycistronic. In another embodiment, the nucleic acid also includes a sequence encoding a reporter protein, e.g., a protein whose abundance can be quantitated and can provide an indication of the quantity of test polypeptide fixed to the plate. The reporter protein can be attached to the test polypeptide, e.g., covalently attached, e.g., attached as a translational fusion. The reporter protein can be an enzyme, e.g., β-galactosidase, chloramphenicol acetyl transferase, β-glucuronidase, and so forth. The reporter protein can produce or modulate light, e.g., a fluorescent protein (e.g., green fluorescent protein, variants thereof, red fluorescent protein, variants thereof, and the like), and luciferase.

The transcription promoter can be a prokaryotic promoter, a eukaryotic promoter, or a viral promoter. In a preferred embodiment, the promoter is the T7 RNA polymerase promoter. The regulatory components, e.g., the transcription promoter, can vary among nucleic acids at different addresses of the plurality. For example, different promoters can be used to vary the amount of polypeptide produced at different addresses.

In one embodiment, the nucleic acid also includes at least one site for recombination, e.g., homologous recombination or site-specific recombination, e.g., a lambda att site or variant thereof; a lox site; or a FLP site. In a preferred embodiment, the recombination site lacks stop codons in the reading frame of a nucleic acid encoding a test amino acid sequence. In another preferred embodiment, the recombination site includes a stop codon in the reading frame of a nucleic acid encoding a test amino acid sequence.

In another embodiment, the nucleic acid includes a sequence encoding a cleavage site, e.g., a protease site, e.g., a site cleaved by a site-specific protease (e.g., a thrombin site, an enterokinase site, a PreScission site, a factor Xa site, or a TEV site), or a chemical cleavage site (e.g., a methionine, preferably a unique methionine (cleavage by cyanogen bromide) or a proline (cleavage by formic acid)).

The nucleic acid can include a sequence encoding a second polypeptide tag in addition to the affinity tag. The second tag can be C-terminal to the test amino acid sequence and the affinity tag can be N-terminal to the test amino acid sequence; the second tag can be N-terminal to the test amino acid sequence, and the affinity tag can be C-terminal to the test amino acid sequence; the second tag and the affinity tag can be adjacent to one another, or separated by a linker sequence, both being N-terminal or C-terminal to the test amino acid sequence. In one embodiment, the second tag is an additional affinity tag, e.g., the same or different from the first tag. In another embodiment, the second tag is a recognition tag. For example, the recognition tag can report the presence and/or amount of test polypeptide at an address. Preferably the recognition tag has a sequence other than the sequence of the affinity tag. In still another embodiment, a plurality of polypeptide tags (e.g., less than 3, 4, 5, about 10, or about 20 tags) are encoded in addition to the first affinity tag. Each polypeptide tag of the plurality can be the same as or different from the first affinity tag.

The nucleic acid sequence can further include an identifier sequence, e.g., a non-coding nucleic acid sequence, e.g., one that is synthetically inserted, and allows for uniquely identifying the nucleic acid sequence. The identifier sequence can be sufficient in length to uniquely identify each sequence in the plurality; e.g., it is about 5 to 500, 10 to 100, 10 to 50, or about 10 to 30 nucleotides in length. The identifier can be selected so that it is not complementary or identical to another identifier or any region of each nucleic acid sequence of the plurality on the array.

The test amino acid sequence can further include a protein splicing sequence or intein. The intein can be inserted in the middle of a test amino acid sequence. The intein can be a naturally-occurring intein or a mutated intein.

The nucleic acid sequences encoding the test amino acid sequences can be obtained from a collection of full-length expressed genes (e.g., a repository of clones), a cDNA library, or a genomic library. The test amino acid sequences can be genes expressed in a tissue, e.g., a normal or diseased tissue. The test polypeptides can be mutants or variants of a scaffold protein (e.g., an antibody, zinc-finger, polypeptide hormone etc.). In yet another embodiment, the test polypeptides are random amino acid sequences, patterned amino acids sequences, or designed amino acids sequences (e.g., sequence designed by manual, rational, or computer-aided approaches). The plurality of test amino acid sequences can include a plurality from a first source, and plurality from a second source. For example, the test amino acid sequences on half the addresses of an array are from a diseased tissue or a first species, whereas the sequences on the remaining half are from a normal tissue or a second species.

In a preferred embodiment, each address of the plurality further includes one or more second nucleic acids, e.g., a plurality of unique nucleic acids. Hence, the plurality in toto can encode a plurality of test sequences. For example, each address of the plurality can encode a pool of test polypeptide sequences, e.g., a subset of a library or clone bank. A second array can be provided in which each address of the plurality of the second array includes a single or subset of members of the pool present at an address of the first array. The first and the second array can be used consecutively.

In other preferred embodiments, each address of the plurality further includes a second nucleic acid encoding a second amino acid sequence.

In one preferred embodiment, each address of the plurality includes a first test amino acid sequence that is common to all addresses of the plurality, and a second test amino acid sequence that is unique among all the addresses of the plurality. For example, the second test amino acid sequences can be query sequences whereas the first amino test amino acid sequence can be a target sequence. In another preferred embodiment, each address of the plurality includes a first test amino acid sequence that is unique among all the addresses of the plurality, and a second test amino acid sequence that is common to all addresses of the plurality. For example, the first test amino acid sequences can be query sequences whereas the second amino test amino acid sequence can be a target sequence. The second nucleic acid encoding the second test amino acid sequence can include a sequence encoding a recognition tag and/or an affinity tag.

At at least one address of the plurality, the first and second amino acid sequences can be such that they interact with one another. In one preferred embodiment, they are capable of binding to each other. The second test amino acid sequence is optionally fused to a detectable amino acid sequence, e.g., an epitope tag, an enzyme, a fluorescent protein (e.g., GFP, BFP, variants thereof). The second test amino acid sequence can be itself detectable (e.g., an antibody is available which specifically recognizes it). The method can further include detecting the second test amino acid sequence at each address of the plurality, e.g., by detecting the detectable amino acid sequence (e.g., the epitope tag, enzyme or fluorescent protein).

In another preferred embodiment, one is capable of modifying the other (e.g., making or breaking a bond, preferably a covalent bond, of the other). For example, the first amino acid sequence is kinase capable of phosphorylating the second amino acid sequence; the first is a methylase capable of methylating the second; the first is a ubiquitin ligase capable of ubiquitinating the second; the first is a protease capable of cleaving the second; and so forth. The method can further include detecting the modification at each address of the plurality.

These embodiments can be used to identify an interaction or to identify a compound that modulates, e.g., inhibits or enhances, an interaction.

The binding agent can be attached to the substrate. For example, the substrate can be derivatized and the binding agent covalent attached thereto. The binding agent can be attached via a bridging moiety, e.g., a specific binding pair. (e.g., the substrate contains a first member of a specific binding pair, and the binding agent is linked to the second member of the binding pair, the second member being attached to the substrate).

In yet another embodiment, an insoluble substrate (e.g., a bead or particle), is disposed at each address of the plurality, and the binding agent is attached to the insoluble substrate. The insoluble substrate can further contain information encoding its identity, e.g., a reference to the address on which it is disposed. The insoluble substrate can be tagged using a chemical tag, or an electronic tag (e.g., a transponder). The insoluble substrate can be disposed such that it can be removed for later analysis.

The method can further include providing a database, e.g., in computer memory or a computer readable medium. Each record of the database can include a field for the amino acid sequence encoded by the nucleic acid sequence and a descriptor or reference for the physical location of the nucleic acid sequence on the array. The database can include a record for each address of the plurality present on the array. Optionally, the method includes entering into the record also includes a field representing a result (e.g., a qualitative or quantitative result) of detecting the polypeptide encoded by the nucleic acid sequence. The method can also further include clustering or grouping the records based on the result.

The invention also features a method of providing an array to a user. The method includes providing the user with a substrate having a plurality of addresses and a vector nucleic acid. The vector nucleic acid can include one or more sites for insertion of a test amino acid sequence (e.g., a recombination site or a restriction site), and a sequence encoding an affinity tag. In a preferred embodiment, the vector nucleic acid has two sites for insertion, and a toxic gene inserted between the two sites. In another embodiment, the sites for insertion are homologous recombination or site-specific recombination sites, e.g., a lambda att site or variant thereof; a lox site; or a FLP site. In a preferred embodiment, one or both recombination sites lack stop codons in the reading frame of a nucleic acid encoding a test amino acid sequence. In another preferred embodiment, one or both recombination sites include a stop codon in the reading frame of a nucleic acid encoding a test amino acid sequence.

In a much preferred embodiment, the affinity tag is in frame with the translation frame of a nucleic acid sequence (e.g., a sequence to be inserted) encoding a test amino acid sequence. In a preferred embodiment, the affinity tag is fused directly to the test amino acid sequence, e.g., directly amino-terminal, or directly carboxy-terminal. In another preferred embodiment, the affinity tag is separated from the test amino acid by one or more linker amino acids, e.g., 1, 2, 3, 4, 5, 6, 8, 10, 12, 20, 30 or more amino acids, preferably about 1 to 20, or about 3 to 12 amino acids. The linker amino acids can include a cleavage site, flexible amino acids (e.g., glycine, alanine, or serine, preferably glycine), and/or polar amino acids. The linker and affinity tag can be amino-terminal or carboxy-terminal to the test amino acid sequence. The cleavage site can be a protease site, e.g., a site cleaved by a site-specific protease (e.g., a thrombin site, an enterokinase site, a PreScission site, a factor Xa site, or a TEV site), or a chemical cleavage site (e.g., a methionine, preferably a unique methionine (cleavage by cyanogen bromide) or a proline (cleavage by formic acid)).

In a preferred embodiment, the method includes providing the user with at least a second vector nucleic acid. The second vector nucleic acid can include one or more sites for insertion of a test amino acid sequence (e.g., a recombination site or a restriction site). In one embodiment, the second vector nucleic acid has a second test amino acid sequence inserted therein. Multiple nucleic acids can be provided, each having a unique test amino acid sequence, e.g., for disposal at a unique address of the substrate. The method can further include contacting each address with a transcription effector and/or a translation effector.

In a preferred embodiment, the second vector nucleic acid has a recognition tag, e.g., an epitope tag, an enzyme, a fluorescent protein (e.g., GFP, BFP, variants thereof).

In a preferred embodiment, each test amino acid sequence in the plurality of addresses is unique. For example, a test amino acid sequence can differ from all other test amino acid sequence of the plurality by 1, or more amino acid differences, (e.g., about 2, 3, 4, 5, 8, 16, 32, 64 or more differences; and, by way of example, has about 800, 256, 128, 64, or 32, 16, 8, 4, or fewer differences). In another preferred embodiment, the test amino acid sequence encoded by the nucleic acid at each address of the plurality is identical to all other test amino acid sequences in the plurality of addresses.

The first and/or second vector nucleic acid can further include one or more of: a transcription promoter; a transcription regulatory sequence; a untranslated leader sequence; a sequence encoding a cleavage site; a recombination site; a 3′ untranslated sequence; a transcriptional terminator; and an internal ribosome entry site. In one embodiment, the nucleic acid sequence includes a plurality of cistrons (also termed “open reading frames”), e.g., the sequence is dicistronic or polycistronic. In another embodiment, the nucleic acid also includes a sequence encoding a reporter protein, e.g., a protein whose abundance can be quantitated and can provide an indication of the quantity of test polypeptide fixed to the plate. The reporter protein can be attached to the test polypeptide, e.g., covalently attached, e.g., attached as a translational fusion. The reporter protein can be an enzyme, e.g., β-galactosidase, chloramphenicol acetyl transferase, β-glucuronidase, and so forth. The reporter protein can produce or modulate light, e.g., a fluorescent protein (e.g., green fluorescent protein, variants thereof, red fluorescent protein, variants thereof, and the like), and luciferase.

The transcription promoter can be a prokaryotic promoter, a eukaryotic promoter, or a viral promoter. In a preferred embodiment, the promoter is the T7 RNA polymerase promoter.

In a preferred embodiment, the method further includes contacting the vector nucleic acid, and optionally the second vector nucleic acid, with a test nucleic acid which includes a nucleic acid encoding a test amino acid sequence so as to insert the test amino acid sequence into the vector nucleic acid. The test nucleic acid can be flanked, e.g., on both ends by a site, e.g., a site compatible with the vector nucleic acid (e.g., having sequence for recombination with a sequence in the vector; or having a restriction site which leaves an overhang or blunt end such that the overhang or blunt end can be ligated into the vector nucleic acid (e.g., the restricted vector nucleic acid)). The contact step can include contacting the vector nucleic acid with a recombinase, a ligase, and/or a restriction endonuclease. For example, the recombinase can mediate recombination, e.g., site-specific recombination or homologous recombination, between a recombination site on the test nucleic acid and a recombination sequence on the vector nucleic acid.

In a preferred embodiment, each address of the plurality has a binding agent capable of recognizing the affinity tag. The binding agent can be attached to the substrate. For example, the substrate can be derivatized and the binding agent covalent attached thereto. The binding agent can be attached via a bridging moiety, e.g., a specific binding pair. (e.g., the substrate contains a first member of a specific binding pair, and the binding agent is linked to the second member of the binding pair, the second member being attached to the substrate).

In yet another embodiment, an insoluble substrate (e.g., a bead or particle), is disposed at each address of the plurality, and the binding agent is attached to the insoluble substrate. The insoluble substrate can further contain information encoding its identity, e.g., a reference to the address on which it is disposed. The insoluble substrate can be tagged using a chemical tag, or an electronic tag (e.g., a transponder). The insoluble substrate can be disposed such that it can be removed for later analysis.

In a preferred embodiment, the method further includes disposing at an address of the plurality a vector nucleic acid that includes a nucleic acid encoding a test amino acid sequence. This step can be repeated until a vector nucleic acid is disposed at each address of the plurality. In embodiments using a second vector nucleic acid in addition to the first, the method can include disposing at each address of the plurality a second vector nucleic acid encoding a different test amino acid sequence from the first vector nucleic acid.

In another preferred embodiment, the method further includes disposing at an address of the plurality a vector nucleic acid that does not include a nucleic acid encoding a test amino acid sequence and concurrently or separately disposing a nucleic acid encoding a test amino acid sequence. This step can be repeated until a vector nucleic acid is disposed at each address of the plurality. The method can also further including contacting each address of the plurality with a recombinase or a ligase.

The first or second vector nucleic acid can include a sequence encoding a second polypeptide tag in addition to the affinity tag. The second tag can be C-terminal to the test amino acid sequence and the affinity tag can be N-terminal to the test amino acid sequence; the second tag can be N-terminal to the test amino acid sequence, and the affinity tag can be C-terminal to the test amino acid sequence; the second tag and the affinity tag can be adjacent to one another, or separated by a linker sequence, both being N-terminal or C-terminal to the test amino acid sequence. In one embodiment, the second tag is an additional affinity tag, e.g., the same or different from the first tag. In another embodiment, the second tag is a recognition tag. For example, the recognition tag can report the presence and/or amount of test polypeptide at an address. Preferably the recognition tag has a sequence other than the sequence of the affinity tag. In still another embodiment, a plurality of polypeptide tags (e.g., less than 3, 4, 5, about 10, or about 20 tags) are encoded in addition to the first affinity tag. Each polypeptide tag of the plurality can be the same as or different from the first affinity tag.

The first or second vector nucleic acid sequence can further include a sequence encoding a protein splicing sequence or intein. The intein can be inserted in the middle of a test amino acid sequence. The intein can be a naturally-occurring intein or a mutated intein.

The nucleic acids encoding the test amino acid sequences can be obtained from a collection of full-length expressed genes (e.g., a repository of clones), a cDNA library, or a genomic library. The encoding nucleic acids can be nucleic acids (e.g., an mRNA or cDNA) expressed in a tissue, e.g., a normal or diseased tissue. The test polypeptides (i.e., test amino acid sequences) can be mutants or variants of a scaffold protein (e.g., an antibody, zinc-finger, polypeptide hormone etc.). In yet another embodiment, the test polypeptides are random amino acid sequences, patterned amino acids sequences, or designed amino acids sequences (e.g., sequence designed by manual, rational, or computer-aided approaches). The plurality of test amino acid sequences can include a plurality from a first source, and plurality from a second source. For example, the test amino acid sequences on half the addresses of an array are from a diseased tissue or a first species, whereas the sequences on the remaining half are from a normal tissue or a second species.

The method can further include detecting the first or the second test amino acid sequence at each address of the plurality.

In another preferred embodiment using a first and a second vector nucleic acid, one test amino acid sequence is capable of modifying the other (e.g., making or breaking a bond, preferably a covalent bond, of the other). For example, the first amino acid sequence is kinase capable of phosphorylating the second amino acid sequence; the first is a methylase capable of methylating the second; the first is a ubiquitin ligase capable of ubiquitinating the second; the first is a protease capable of cleaving the second; and so forth. The method can further include detecting the modification at each address of the plurality.

These embodiments can be used to identify an interaction or to identify a compound that modulates, e.g., inhibits or enhances, an interaction.

In another aspect, the invention features a method of providing an array of polypeptides. The method includes: (1) providing or obtaining a substrate with a plurality of addresses, each address of the plurality including (i) a nucleic acid encoding an amino acid sequence comprising a test amino acid sequence and an affinity tag, and (ii) a binding agent that recognizes the affinity tag; (2) contacting each address of the plurality with a translation effector to thereby translate the hybrid amino acid sequence; and (3) maintaining the substrate under conditions permissive for the amino acid sequence to bind the binding agent.

In one embodiment, the nucleic acid provided on the substrate is synthesized in situ, e.g., by light-directed chemistry. In another embodiment, each address of the plurality is provided with a nucleic acid, e.g., by pipetting, spotting, printing (e.g., with pins), piezoelectric delivery, or, e.g., other means of mechanical delivery. In a preferred embodiment, the provided nucleic acid is a template nucleic acid, and the method further includes amplifying the template, e.g., by PCR, NASBA, or RCA. The method can further include transcribing the nucleic acid to produce one or more RNA molecules encoding the test amino acid sequence.

The method can further include washing the substrate, e.g., after sufficient contact with a translation effector. The wash step can be repeated, e.g., one or more times, e.g., until a translation effector or translation effector component is removed. The wash step can remove unbound proteins. The stringency of the wash step can vary, e.g., the salt, pH, and buffer composition of the wash buffer can vary. For example, if the translated test polypeptide is covalently captured, or captured by an interaction resistant to chaotropes (e.g., binding of a 6-histidine motif to Ni²⁺.NTA), the substrate can be washed with a chaotrope, (e.g., guanidinium hydrochloride, or urea). In a subsequent step, the chaotrope can itself be washed from the array, and the polypeptides renatured.

In one embodiment, the nucleic acid sequence also encodes a cleavage site, e.g., a protease site, e.g., between the test amino acid sequence and the affinity tag. The method can further include contacting an address of the array with a protease that specifically recognizes the site.

The method can further include contacting the substrate with a second substrate. For example, in an embodiment wherein the substrate is a gel, the gel can be contacted with a second gel, and the contents of one gel can be transferred to another (e.g., by diffusion or electrophoresis). The method can include disrupting the binding between the affinity tag and the binding agent or between the binding agent and the substrate prior to transfer.

The method can further include contacting the substrate with living cells, and detecting an address wherein a parameter of the cell is altered relative to another address.

In a preferred embodiment, each test amino acid sequence in the plurality of addresses is unique. For example, a test amino acid sequence can differ from all other test amino acid sequence of the plurality by 1, or more amino acid differences, (e.g., about 2, 3, 4, 5, 8, 16, 32, 64 or more differences; and, by way of example, has about 800, 256, 128, 64, or 32, 16, 8, 4, or fewer differences). In another preferred embodiment, the test amino acid sequence encoded by the nucleic acid at each address of the plurality is identical to all other test amino acid sequences in the plurality of addresses. In a preferred embodiment, the affinity tag encoded by the nucleic acid at each address of the plurality is the same, or substantially identical to all other affinity tags in the plurality of addresses. In another preferred embodiment, the nucleic acid at each address of the plurality encodes more than one affinity tag. In yet another preferred embodiment, the affinity tag encoded by the nucleic acid at an address of the plurality differs from at least one other affinity tag in the plurality of addresses.

In a preferred embodiment, the affinity tag is fused directly to the test amino acid sequence, e.g., directly amino-terminal, or directly carboxy-terminal. In another preferred embodiment, the affinity tag is separated from the test amino acid by one or more linker amino acids, e.g., 1, 2, 3, 4, 5, 6, 8, 10, 12, 20, 30 or more amino acids, preferably about 1 to 20, or about 3 to 12 amino acids. The linker amino acids can include a cleavage site, flexible amino acids (e.g., glycine, alanine, or serine, preferably glycine), and/or polar amino acids. The linker and affinity tag can be amino-terminal or carboxy-terminal to the test amino acid sequence.

The nucleic acid can further include one or more of: a transcription promoter; a transcription regulatory sequence; a untranslated leader sequence; a sequence encoding a cleavage site; a recombination site; a 3′ untranslated sequence; a transcriptional terminator; and an internal ribosome entry site. In one embodiment, the nucleic acid sequence includes a plurality of cistrons (also termed “open reading frames”), e.g., the sequence is dicistronic or polycistronic. In another embodiment, the nucleic acid also includes a sequence encoding a reporter protein, e.g., a protein whose abundance can be quantitated and can provide an indication of the quantity of test polypeptide fixed to the plate. The reporter protein can be attached to the test polypeptide, e.g., covalently attached, e.g., attached as a translational fusion. The reporter protein can be an enzyme, e.g., β-galactosidase, chloramphenicol acetyl transferase, β-glucuronidase, and so forth. The reporter protein can produce or modulate light, e.g., a fluorescent protein (e.g., green fluorescent protein, variants thereof, red fluorescent protein, variants thereof, and the like), and luciferase.

The transcription promoter can be a prokaryotic promoter, a eukaryotic promoter, or a viral promoter. In a preferred embodiment, the promoter is the T7 RNA polymerase promoter. The regulatory components, e.g., the transcription promoter, can vary among nucleic acids at different addresses of the plurality. For example, different promoters can be used to vary the amount of polypeptide produced at different addresses.

In one embodiment, the nucleic acid also includes at least one site for recombination, e.g., homologous recombination or site-specific recombination, e.g., a lambda att site or variant thereof; a lox site; or a FLP site. In a preferred embodiment, the recombination site lacks stop codons in the reading frame of a nucleic acid encoding a test amino acid sequence. In another preferred embodiment, the recombination site includes a stop codon in the reading frame of a nucleic acid encoding a test amino acid sequence.

In another embodiment, the nucleic acid includes a sequence encoding a cleavage site, e.g., a protease site, e.g., a site cleaved by a site-specific protease (e.g., a thrombin site, an enterokinase site, a PreScission site, a factor Xa site, or a TEV site), or a chemical cleavage site (e.g., a methionine, preferably a unique methionine (cleavage by cyanogen bromide) or a proline (cleavage by formic acid)).

The nucleic acid can include a sequence encoding a second polypeptide tag in addition to the affinity tag. The second tag can be C-terminal to the test amino acid sequence and the affinity tag can be N-terminal to the test amino acid sequence; the second tag can be N-terminal to the test amino acid sequence, and the affinity tag can be C-terminal to the test amino acid sequence; the second tag and the affinity tag can be adjacent to one another, or separated by a linker sequence, both being N-terminal or C-terminal to the test amino acid sequence. In one embodiment, the second tag is an additional affinity tag, e.g., the same or different from the first tag. In another embodiment, the second tag is a recognition tag. For example, the recognition tag can report the presence and/or amount of test polypeptide at an address. Preferably the recognition tag has a sequence other than the sequence of the affinity tag. In still another embodiment, a plurality of polypeptide tags (e.g., less than 3, 4, 5, about 10, or about 20 tags) are encoded in addition to the first affinity tag. Each polypeptide tag of the plurality can be the same as or different from the first affinity tag.

The nucleic acid sequence can further include an identifier sequence, e.g., a non-coding nucleic acid sequence, e.g., one that is synthetically inserted, and allows for uniquely identifying the nucleic acid sequence. The identifier sequence can be sufficient in length to uniquely identify each sequence in the plurality; e.g., it is about 5 to 500, 10 to 100, 10 to 50, or about 10 to 30 nucleotides in length. The identifier can be selected so that it is not complementary or identical to another identifier or any region of each nucleic acid sequence of the plurality on the array.

The test amino acid sequence can further include a protein splicing sequence or intein. The intein can be inserted in the middle of a test amino acid sequence. The intein can be a naturally-occurring intein or a mutated intein.

The nucleic acid sequences encoding the test amino acid sequences can be obtained from a collection of full-length expressed genes (e.g., a repository of clones), a cDNA library, or a genomic library. The test amino acid sequences can be genes expressed in a tissue, e.g., a normal or diseased tissue. The test polypeptides can be mutants or variants of a scaffold protein (e.g., an antibody, zinc-finger, polypeptide hormone etc.). In yet another embodiment, the test polypeptides are random amino acid sequences, patterned amino acids sequences, or designed amino acids sequences (e.g., sequence designed by manual, rational, or computer-aided approaches). The plurality of test amino acid sequences can include a plurality from a first source, and plurality from a second source. For example, the test amino acid sequences on half the addresses of an array are from a diseased tissue or a first species, whereas the sequences on the remaining half are from a normal tissue or a second species.

In a preferred embodiment, each address of the plurality further includes one or more second nucleic acids, e.g., a plurality of unique nucleic acids. Hence, the plurality in toto can encode a plurality of test sequences. For example, each address of the plurality can encode a pool of test polypeptide sequences, e.g., a subset of a library or clone bank. A second array can be provided in which each address of the plurality of the second array includes a single or subset of members of the pool present at an address of the first array. The first and the second array can be used consecutively.

In other preferred embodiments, each address of the plurality further includes a second nucleic acid encoding a second amino acid sequence.

In one preferred embodiment, each address of the plurality includes a first test amino acid sequence that is common to all addresses of the plurality, and a second test amino acid sequence that is unique among all the addresses of the plurality. For example, the second test amino acid sequences can be query sequences whereas the first amino test amino acid sequence can be a target sequence. In another preferred embodiment, each address of the plurality includes a first test amino acid sequence that is unique among all the addresses of the plurality, and a second test amino acid sequence that is common to all addresses of the plurality. For example, the first test amino acid sequences can be query sequences whereas the second amino test amino acid sequence can be a target sequence. The second nucleic acid encoding the second test amino acid sequence can include a sequence encoding a recognition tag and/or an affinity tag.

At at least one address of the plurality, the first and second amino acid sequences can be such that they interact with one another. In one preferred embodiment, they are capable of binding to each other. The second test amino acid sequence is optionally fused to a detectable amino acid sequence, e.g., an epitope tag, an enzyme, a fluorescent protein (e.g., GFP, BFP, variants thereof). The second test amino acid sequence can be itself detectable (e.g., an antibody is available which specifically recognizes it). The method can further include detecting the second test amino acid sequence at each address of the plurality, e.g., by detecting the detectable amino acid sequence (e.g., the epitope tag, enzyme or fluorescent protein).

In another preferred embodiment, one is capable of modifying the other (e.g., making or breaking a bond, preferably a covalent bond, of the other). For example, the first amino acid sequence is kinase capable of phosphorylating the second amino acid sequence; the first is a methylase capable of methylating the second; the first is a ubiquitin ligase capable of ubiquitinating the second; the first is a protease capable of cleaving the second; and so forth. The method can further include detecting the modification at each address of the plurality.

These embodiments can be used to identify an interaction or to identify a compound that modulates, e.g., inhibits or enhances, an interaction.

The binding agent can be attached to the substrate. For example, the substrate can be derivatized and the binding agent covalent attached thereto. The binding agent can be attached via a bridging moiety, e.g., a specific binding pair. (e.g., the substrate contains a first member of a specific binding pair, and the binding agent is linked to the second member of the binding pair, the second member being attached to the substrate). In yet another embodiment, an insoluble substrate (e.g., a bead or particle), is disposed at each address of the plurality, and the binding agent is attached to the insoluble substrate. The insoluble substrate can further contain information encoding its identity, e.g., a reference to the address on which it is disposed. The insoluble substrate can be tagged using a chemical tag, or an electronic tag (e.g., a transponder). The insoluble substrate can be disposed such that it can be removed for later analysis.

In another aspect, the invention features a method of evaluating, e.g., identifying a polypeptide-polypeptide interaction. The method includes: (1) providing or obtaining a substrate with a plurality of addresses, each address of the plurality comprising (i) a first nucleic acid encoding an amino acid sequence comprising a first amino acid sequence and an affinity tag, (ii) a binding agent that recognizes the affinity tag, and (iii) a second nucleic acid encoding a second amino acid sequence; (2) contacting each address of the plurality with a translation effector to thereby translate the first nucleic acid and the second nucleic acid to synthesize the first and second amino acid sequences; and optionally (3) maintaining the substrate under conditions permissive for the hybrid amino acid sequence to bind binding agent.

In one preferred embodiment, the first amino acid sequence is common to all addresses of the plurality, and a second test amino acid sequence is unique among all the addresses of the plurality. For example, the second test amino acid sequences can be query sequences whereas the first amino test amino acid sequence can be a target sequence. In another preferred embodiment, the first amino acid sequence is unique among all the addresses of the plurality, and the second amino acid sequence is common to all addresses of the plurality. For example, the first test amino acid sequences can be query sequences whereas the second amino test amino acid sequence can be a target sequence. The second nucleic acid encoding the second test amino acid sequence can include a sequence encoding a recognition tag and/or an affinity tag.

The method can further include detecting the presence of the second amino acid sequence at each of the plurality of addresses.

In one preferred embodiment, the second nucleic acid sequence also encodes a polypeptide tag. The polypeptide tag can be an epitope (e.g., recognized by a monoclonal antibody), or a binding agent (e.g., avidin or streptavidin, GST, or chitin binding protein). The detection of the second amino acid sequence can entail contacting each address of the plurality with a binding agent, e.g., a labeled biotin moiety, labeled glutathione, labeled chitin, a labeled antibody, etc. In another embodiment, each address of the plurality is contacted with an antibody specific to the second amino acid sequence.

In another preferred embodiment, the second nucleic acid sequence includes a recognition tag. The recognition tag can be an epitope tag, enzyme or fluorescent protein. Examples of enzymes include horseradish peroxidase, alkaline phosphatase, luciferase, or cephalosporinase. The method can further include contacting each address of the plurality with an appropriate cofactor and/or substrate for the enzyme. Examples of fluorescent proteins include green fluorescent protein (GFP), and variants thereof, e.g., enhanced GFP, blue fluorescent protein (BFP), cyan FP, etc. The detection of the second amino acid sequence can entail monitoring fluorescence, assessing enzyme activity, measuring an added binding agent, e.g., a labeled biotin moiety, a labeled antibody, etc.

In another preferred embodiment, one is capable of modifying the other (e.g., making or breaking a bond, preferably a covalent bond, of the other). For example, the first amino acid sequence is kinase capable of phosphorylating the second amino acid sequence; the first is a methylase capable of methylating the second; the first is a ubiquitin ligase capable of ubiquitinating the second; the first is a protease capable of cleaving the second; and so forth. The method can further include detecting the modification at each address of the plurality.

These embodiments can be used to identify an interaction or to identify a compound that modulates, e.g., inhibits or enhances, an interaction. For example, the method can further include contacting each address of the plurality with a compound, e.g., a small organic molecule, a polypeptide, or a nucleic acid to thereby determine if the compound alters the interaction between the first and second amino acid.

In one preferred embodiment, the first amino acid sequence is a drug candidate, e.g. a random peptide, a randomized or mutated scaffold protein, or a secreted protein (e.g., a cell surface protein, an ectodomain of a transmembrane protein, an antibody, or a polypeptide hormone); and the second amino acid sequence is a drug target. A first amino acid sequence at an address where an interaction between the first amino acid sequence and the second amino acid is detected can be used as a candidate amino acid sequence for additional refinement or as a drug. The first amino acid sequence can be administered to a subject. A nucleic acid encoding the first amino acid sequence can be administered to a subject. In a related preferred embodiment, the first amino acid sequence is the drug target, and the second amino acid sequence is the drug candidate.

In a preferred embodiment, each first amino acid sequence in the plurality of addresses is unique. For example, a first amino acid sequence can differ from all other test amino acid sequence of the plurality by 1, or more amino acid differences, (e.g., about 2, 3, 4, 5, 8, 16, 32, 64 or more differences; and, by way of example, has about 800, 256, 128, 64, or 32, 16, 8, 4, or fewer differences). In another preferred embodiment, the first amino acid sequence encoded by the nucleic acid at each address of the plurality is identical to all other first amino acid sequences in the plurality of addresses. In a preferred embodiment, the affinity tag encoded by the first nucleic acid at each address of the plurality is the same, or substantially identical to all other affinity tags in the plurality of addresses. In another preferred embodiment, the first nucleic acid at each address of the plurality encodes more than one affinity tag. In yet another preferred embodiment, the affinity tag encoded by the first nucleic acid at an address of the plurality differs from at least one other affinity tag in the plurality of addresses.

In a preferred embodiment, the affinity tag is fused directly to the test amino acid sequence, e.g., directly amino-terminal, or directly carboxy-terminal. In another preferred embodiment, the affinity tag is separated from the test amino acid by one or more linker amino acids, e.g., 1, 2, 3, 4, 5, 6, 8, 10, 12, 20, 30 or more amino acids, preferably about 1 to 20, or about 3 to 12 amino acids. The linker amino acids can include a cleavage site, flexible amino acids (e.g., glycine, alanine, or serine, preferably glycine), and/or polar amino acids. The linker and affinity tag can be amino-terminal or carboxy-terminal to the test amino acid sequence.

The first and/or second nucleic acid can be a RNA, or a DNA (e.g., a single-stranded DNA, or a double stranded DNA). In a preferred embodiment, the first and/or second nucleic acid includes a plasmid DNA or a fragment thereof; an amplification product (e.g., a product generated by RCA, PCR, NASBA); or a synthetic DNA.

The first and/or second nucleic acid can further include one or more of: a transcription promoter; a transcription regulatory sequence; a untranslated leader sequence; a sequence encoding a cleavage site; a recombination site; a 3′ untranslated sequence; a transcriptional terminator; and an internal ribosome entry site. In one embodiment, the nucleic acid sequence includes a plurality of cistrons (also termed “open reading frames”), e.g., the sequence is dicistronic or polycistronic. In another embodiment, the nucleic acid also includes a sequence encoding a reporter protein, e.g., a protein whose abundance can be quantitated and can provide an indication of the quantity of test polypeptide fixed to the plate. The reporter protein can be attached to the test polypeptide, e.g., covalently attached, e.g., attached as a translational fusion. The reporter protein can be an enzyme, e.g., β-galactosidase, chloramphenicol acetyl transferase, β-glucuronidase, and so forth. The reporter protein can produce or modulate light, e.g., a fluorescent protein (e.g., green fluorescent protein, variants thereof, red fluorescent protein, variants thereof, and the like), and luciferase.

The transcription promoter can be a prokaryotic promoter, a eukaryotic promoter, or a viral promoter. In a preferred embodiment, the promoter is the T7 RNA polymerase promoter. The regulatory components, e.g., the transcription promoter, can vary among nucleic acids at different addresses of the plurality. For example, different promoters can be used to vary the amount of polypeptide produced at different addresses.

In one embodiment, the first and/or second nucleic acid also includes at least one site for recombination, e.g., homologous recombination or site-specific recombination, e.g., a lambda att site or variant thereof; a lox site; or a FLP site. In a preferred embodiment, the recombination site lacks stop codons in the reading frame of a nucleic acid encoding a test amino acid sequence. In another preferred embodiment, the recombination site includes a stop codon in the reading frame of a nucleic acid encoding a test amino acid sequence.

In another embodiment, the first and/or second nucleic acid includes a sequence encoding a cleavage site, e.g., a protease site, e.g., a site cleaved by a site-specific protease (e.g., a thrombin site, an enterokinase site, a PreScission site, a factor Xa site, or a TEV site), or a chemical cleavage site (e.g., a methionine, preferably a unique methionine (cleavage by cyanogen bromide) or a proline (cleavage by formic acid)).

The first nucleic acid can include a sequence encoding a second polypeptide tag in addition to the affinity tag. The second tag can be C-terminal to the test amino acid sequence and the affinity tag can be N-terminal to the test amino acid sequence; the second tag can be N-terminal to the test amino acid sequence, and the affinity tag can be C-terminal to the test amino acid sequence; the second tag and the affinity tag can be adjacent to one another, or separated by a linker sequence, both being N-terminal or C-terminal to the test amino acid sequence. In one embodiment, the second tag is an additional affinity tag, e.g., the same or different from the first tag. In another embodiment, the second tag is a recognition tag. For example, the recognition tag can report the presence and/or amount of test polypeptide at an address. Preferably the recognition tag has a sequence other than the sequence of the affinity tag. In still another embodiment, a plurality of polypeptide tags (e.g., less than 3, 4, 5, about 10, or about 20 tags) are encoded in addition to the first affinity tag. Each polypeptide tag of the plurality can be the same as or different from the first affinity tag.

The first and/or second nucleic acid sequence can further include an identifier sequence, e.g., a non-coding nucleic acid sequence, e.g., one that is synthetically inserted, and allows for uniquely identifying the nucleic acid sequence. The identifier sequence can be sufficient in length to uniquely identify each sequence in the plurality; e.g., it is about 5 to 500, 10 to 100, 10 to 50, or about 10 to 30 nucleotides in length. The identifier can be selected so that it is not complementary or identical to another identifier or any region of each nucleic acid sequence of the plurality on the array.

The first and/or second amino acid sequence can further include a protein splicing sequence or intein. The intein can be inserted in the middle of a test amino acid sequence. The intein can be a naturally-occurring intein or a mutated intein.

The first and/or second nucleic acid sequences encoding the first and/or second amino acid sequences can be obtained from a collection of full-length expressed genes (e.g., a repository of clones), a cDNA library, or a genomic library. The first and/or second nucleic acid sequences can be nucleic acids expressed in a tissue, e.g., a normal or diseased tissue. The first and/or second amino acid sequences can be mutants or variants of a scaffold protein (e.g., an antibody, zinc-finger, polypeptide hormone etc.). In yet another embodiment, they are random amino acid sequences, patterned amino acids sequences, or designed amino acids sequences (e.g., sequence designed by manual, rational, or computer-aided approaches).

The binding agent can be attached to the substrate. For example, the substrate can be derivatized and the binding agent covalent attached thereto. The binding agent can be attached via a bridging moiety, e.g., a specific binding pair. (e.g., the substrate contains a first member of a specific binding pair, and the binding agent is linked to the second member of the binding pair, the second member being attached to the substrate).

In yet another embodiment, an insoluble substrate (e.g., a bead or particle), is disposed at each address of the plurality, and the binding agent is attached to the insoluble substrate. The insoluble substrate can further contain information encoding its identity, e.g., a reference to the address on which it is disposed. The insoluble substrate can be tagged using a chemical tag, or an electronic tag (e.g., a transponder). The insoluble substrate can be disposed such that it can be removed for later analysis.

In another aspect, the invention features a method of evaluating, e.g., identifying a polypeptide-polypeptide interaction. The method includes: (1) providing or obtaining an array made by the following process: (A) providing or obtaining a substrate with a plurality of addresses, each address having a binding agent that recognizes an affinity tag; (B) disposing in or on each address of the plurality (i) a first nucleic acid encoding an amino acid sequence comprising a first amino acid sequence and the affinity tag, and (ii) a second nucleic acid encoding a second amino acid sequence; and, optionally, (C) contacting each address of the plurality with a translation effector to thereby translate the first and second nucleic acid.

The method can further include maintaining the substrate under conditions permissive for the hybrid amino acid sequence to bind binding agent. The method can further include detecting the presence of the second amino acid sequence at each of the plurality of addresses.

In one preferred embodiment, the first amino acid sequence is common to all addresses of the plurality, and a second test amino acid sequence is unique among all the addresses of the plurality. For example, the second test amino acid sequences can be query sequences whereas the first amino test amino acid sequence can be a target sequence. In another preferred embodiment, the first amino acid sequence is unique among all the addresses of the plurality, and the second amino acid sequence is common to all addresses of the plurality. For example, the first test amino acid sequences can be query sequences whereas the second amino test amino acid sequence can be a target sequence. The second nucleic acid encoding the second test amino acid sequence can include a sequence encoding a recognition tag and/or an affinity tag.

The method can further include detecting the presence of the second amino acid sequence at each of the plurality of addresses.

In one preferred embodiment, the second nucleic acid sequence also encodes a polypeptide tag. The polypeptide tag can be an epitope (e.g., recognized by a monoclonal antibody), or a binding agent (e.g., avidin or streptavidin, GST, or chitin binding protein). The detection of the second amino acid sequence can entail contacting each address of the plurality with a binding agent, e.g., a labeled biotin moiety, labeled glutathione, labeled chitin, a labeled antibody, etc. In another embodiment, each address of the plurality is contacted with an antibody specific to the second amino acid sequence.

In another preferred embodiment, the second nucleic acid sequence includes a recognition tag. The recognition tag can be an epitope tag, enzyme or fluorescent protein. Examples of enzymes include horseradish peroxidase, alkaline phosphatase, luciferase, or cephalosporinase. The method can further include contacting each address of the plurality with an appropriate cofactor and/or substrate for the enzyme. Examples of fluorescent proteins include green fluorescent protein (GFP), and variants thereof, e.g., enhanced GFP, blue fluorescent protein (BFP), cyan FP, etc. The detection of the second amino acid sequence can entail monitoring fluorescence, assessing enzyme activity, measuring an added binding agent, e.g., a labeled biotin moiety, a labeled antibody, etc.

In another preferred embodiment, one is capable of modifying the other (e.g., making or breaking a bond, preferably a covalent bond, of the other). For example, the first amino acid sequence is kinase capable of phosphorylating the second amino acid sequence; the first is a methylase capable of methylating the second; the first is a ubiquitin ligase capable of ubiquitinating the second; the first is a protease capable of cleaving the second; and so forth. The method can further include detecting the modification at each address of the plurality.

These embodiments can be used to identify an interaction or to identify a compound that modulates, e.g., inhibits or enhances, an interaction. For example, the method can further include contacting each address of the plurality with a compound, e.g., a small organic molecule, a polypeptide, or a nucleic acid to thereby determine if the compound alters the interaction between the first and second amino acid.

In one preferred embodiment, the first amino acid sequence is a drug candidate, e.g. a random peptide, a randomized or mutated scaffold protein, or a secreted protein (e.g., a cell surface protein, an ectodomain of a transmembrane protein, an antibody, or a polypeptide hormone); and the second amino acid sequence is a drug target. A first amino acid sequence at an address where an interaction between the first amino acid sequence and the second amino acid is detected can be used as a candidate amino acid sequence for additional refinement or as a drug. The first amino acid sequence can be administered to a subject. A nucleic acid encoding the first amino acid sequence can be administered to a subject. In a related preferred embodiment, the first amino acid sequence is the drug target, and the second amino acid sequence is the drug candidate.

In a preferred embodiment, each first amino acid sequence in the plurality of addresses is unique. For example, a first amino acid sequence can differ from all other test amino acid sequence of the plurality by 1, or more amino acid differences, (e.g., about 2, 3, 4, 5, 8, 16, 32, 64 or more differences; and, by way of example, has about 800, 256, 128, 64, or 32, 16, 8, 4, or fewer differences). In another preferred embodiment, the first amino acid sequence encoded by the nucleic acid at each address of the plurality is identical to all other first amino acid sequences in the plurality of addresses. In a preferred embodiment, the affinity tag encoded by the first nucleic acid at each address of the plurality is the same, or substantially identical to all other affinity tags in the plurality of addresses. In another preferred embodiment, the first nucleic acid at each address of the plurality encodes more than one affinity tag. In yet another preferred embodiment, the affinity tag encoded by the first nucleic acid at an address of the plurality differs from at least one other affinity tag in the plurality of addresses.

In a preferred embodiment, the affinity tag is fused directly to the test amino acid sequence, e.g., directly amino-terminal, or directly carboxy-terminal. In another preferred embodiment, the affinity tag is separated from the test amino acid by one or more linker amino acids, e.g., 1, 2, 3, 4, 5, 6, 8, 10, 12, 20, 30 or more amino acids, preferably about 1 to 20, or about 3 to 12 amino acids. The linker amino acids can include a cleavage site, flexible amino acids (e.g., glycine, alanine, or serine, preferably glycine), and/or polar amino acids. The linker and affinity tag can be amino-terminal or carboxy-terminal to the test amino acid sequence.

The first and/or second nucleic acid can be a RNA, or a DNA (e.g., a single-stranded DNA, or a double stranded DNA). In a preferred embodiment, the first and/or second nucleic acid includes a plasmid DNA or a fragment thereof; an amplification product (e.g., a product generated by RCA, PCR, NASBA); or a synthetic DNA.

The first and/or second nucleic acid can further include one or more of: a transcription promoter; a transcription regulatory sequence; a untranslated leader sequence; a sequence encoding a cleavage site; a recombination site; a 3′ untranslated sequence; a transcriptional terminator; and an internal ribosome entry site. In one embodiment, the nucleic acid sequence includes a plurality of cistrons (also termed “open reading frames”), e.g., the sequence is dicistronic or polycistronic. In another embodiment, the nucleic acid also includes a sequence encoding a reporter protein, e.g., a protein whose abundance can be quantitated and can provide an indication of the quantity of test polypeptide fixed to the plate. The reporter protein can be attached to the test polypeptide, e.g., covalently attached, e.g., attached as a translational fusion. The reporter protein can be an enzyme, e.g., β-galactosidase, chloramphenicol acetyl transferase, β-glucuronidase, and so forth. The reporter protein can produce or modulate light, e.g., a fluorescent protein (e.g., green fluorescent protein, variants thereof, red fluorescent protein, variants thereof, and the like), and luciferase.

The transcription promoter can be a prokaryotic promoter, a eukaryotic promoter, or a viral promoter. In a preferred embodiment, the promoter is the T7 RNA polymerase promoter. The regulatory components, e.g., the transcription promoter, can vary among nucleic acids at different addresses of the plurality. For example, different promoters can be used to vary the amount of polypeptide produced at different addresses.

In one embodiment, the first and/or second nucleic acid also includes at least one site for recombination, e.g., homologous recombination or site-specific recombination, e.g., a lambda att site or variant thereof; a lox site; or a FLP site. In a preferred embodiment, the recombination site lacks stop codons in the reading frame of a nucleic acid encoding a test amino acid sequence. In another preferred embodiment, the recombination site includes a stop codon in the reading frame of a nucleic acid encoding a test amino acid sequence.

In another embodiment, the first and/or second nucleic acid includes a sequence encoding a cleavage site, e.g., a protease site, e.g., a site cleaved by a site-specific protease (e.g., a thrombin site, an enterokinase site, a PreScission site, a factor Xa site, or a TEV site), or a chemical cleavage site (e.g., a methionine, preferably a unique methionine (cleavage by cyanogen bromide) or a proline (cleavage by formic acid)).

The first nucleic acid can include a sequence encoding a second polypeptide tag in addition to the affinity tag. The second tag can be C-terminal to the test amino acid sequence and the affinity tag can be N-terminal to the test amino acid sequence; the second tag can be N-terminal to the test amino acid sequence, and the affinity tag can be C-terminal to the test amino acid sequence; the second tag and the affinity tag can be adjacent to one another, or separated by a linker sequence, both being N-terminal or C-terminal to the test amino acid sequence. In one embodiment, the second tag is an additional affinity tag, e.g., the same or different from the first tag. In another embodiment, the second tag is a recognition tag. For example, the recognition tag can report the presence and/or amount of test polypeptide at an address. Preferably the recognition tag has a sequence other than the sequence of the affinity tag. In still another embodiment, a plurality of polypeptide tags (e.g., less than 3, 4, 5, about 10, or about 20 tags) are encoded in addition to the first affinity tag. Each polypeptide tag of the plurality can be the same as or different from the first affinity tag.

The first and/or second nucleic acid sequence can further include an identifier sequence, e.g., a non-coding nucleic acid sequence, e.g., one that is synthetically inserted, and allows for uniquely identifying the nucleic acid sequence. The identifier sequence can be sufficient in length to uniquely identify each sequence in the plurality; e.g., it is about 5 to 500, 10 to 100, 10 to 50, or about 10 to 30 nucleotides in length. The identifier can be selected so that it is not complementary or identical to another identifier or any region of each nucleic acid sequence of the plurality on the array.

The first and/or second amino acid sequence can further include a protein splicing sequence or intein. The intein can be inserted in the middle of a test amino acid sequence. The intein can be a naturally-occurring intein or a mutated intein.

The first and/or second nucleic acid sequences encoding the first and/or second amino acid sequences can be obtained from a collection of full-length expressed genes (e.g., a repository of clones), a cDNA library, or a genomic library. The first and/or second nucleic acid sequences can be nucleic acids expressed in a tissue, e.g., a normal or diseased tissue. The first and/or second amino acid sequences can be mutants or variants of a scaffold protein (e.g., an antibody, zinc-finger, polypeptide hormone etc.). In yet another embodiment, they are random amino acid sequences, patterned amino acids sequences, or designed amino acids sequences (e.g., sequence designed by manual, rational, or computer-aided approaches).

The binding agent can be attached to the substrate. For example, the substrate can be derivatized and the binding agent covalent attached thereto. The binding agent can be attached via a bridging moiety, e.g., a specific binding pair. (e.g., the substrate contains a first member of a specific binding pair, and the binding agent is linked to the second member of the binding pair, the second member being attached to the substrate).

In yet another embodiment, an insoluble substrate (e.g., a bead or particle), is disposed at each address of the plurality, and the binding agent is attached to the insoluble substrate. The insoluble substrate can further contain information encoding its identity, e.g., a reference to the address on which it is disposed. The insoluble substrate can be tagged using a chemical tag, or an electronic tag (e.g., a transponder). The insoluble substrate can be disposed such that it can be removed for later analysis.

In another aspect, the method features a method of evaluating, e.g., identifying, a polypeptide-polypeptide interaction. The method includes: (1) providing or obtaining an array made by the following production method: (A) providing or obtaining a substrate with a plurality of addresses, each address of the plurality comprising (i) a first nucleic acid encoding a hybrid amino acid sequence comprising a first amino acid sequence and an affinity tag, (ii) a binding agent that recognizes the affinity tag, and (iii) a second nucleic acid encoding a second amino acid sequence; and (B) contacting each address of the plurality with a translation effector to thereby translate the first and second nucleic acid sequences. The evaluation method further includes: (2) at each of the plurality of addresses, detecting at least one parameter selected from the group consisting of: (i) the proximity of the second amino acid sequence to the first amino acid sequence; (ii) the proximity of the second amino acid sequence to the substrate or a compound bound thereto; (iii) the rotational freedom of the second amino acid sequence; and (iv) the refractive index of the substrate. The evaluation method can optionally include, e.g., prior to the detecting step, (3) maintaining the substrate under conditions permissive for the hybrid amino acid sequence to bind binding agent.

The method can further include washing the substrate prior to the detection step. The stringency of the wash step can be adjusted in order to remove the translation effector, and non-specifically bound proteins.

In one preferred embodiment, the first amino acid sequence is common to all addresses of the plurality, and a second test amino acid sequence is unique among all the addresses of the plurality. For example, the second test amino acid sequences can be query sequences whereas the first amino test amino acid sequence can be a target sequence. In another preferred embodiment, the first amino acid sequence is unique among all the addresses of the plurality, and the second amino acid sequence is common to all addresses of the plurality. For example, the first test amino acid sequences can be query sequences whereas the second amino test amino acid sequence can be a target sequence. The second nucleic acid encoding the second test amino acid sequence can include a sequence encoding a recognition tag and/or an affinity tag.

The method can further include detecting the presence of the second amino acid sequence at each of the plurality of addresses.

In one preferred embodiment, the second nucleic acid sequence also encodes a polypeptide tag. The polypeptide tag can be an epitope (e.g., recognized by a monoclonal antibody), or a binding agent (e.g., avidin or streptavidin, GST, or chitin binding protein). The detection of the second amino acid sequence can entail contacting each address of the plurality with a binding agent, e.g., a labeled biotin moiety, labeled glutathione, labeled chitin, a labeled antibody, etc. In another embodiment, each address of the plurality is contacted with an antibody specific to the second amino acid sequence. The antibody can be labeled, e.g., with a fluorophore.

In another preferred embodiment, the second nucleic acid sequence includes a recognition tag. The recognition tag can be an epitope tag, enzyme or fluorescent protein. Examples of enzymes include horseradish peroxidase, alkaline phosphatase, luciferase, or cephalosporinase. The method can further include contacting each address of the plurality with an appropriate cofactor and/or substrate for the enzyme. Examples of fluorescent proteins include green fluorescent protein (GFP), and variants thereof, e.g., enhanced GFP, blue fluorescent protein (BFP), cyan FP, etc.

The method can further include contacting each address of the plurality with a compound, e.g., a small organic molecule, a polypeptide, or a nucleic acid to thereby determine if the compound alters the interaction between the first and second amino acid.

In one preferred embodiment, the first amino acid sequence is a drug candidate, e.g. a random peptide, a randomized or mutated scaffold protein, or a secreted protein (e.g., a cell surface protein, an ectodomain of a transmembrane protein, an antibody, or a polypeptide hormone); and the second amino acid sequence is a drug target. A first amino acid sequence at an address where an interaction between the first amino acid sequence and the second amino acid is detected can be used as a candidate amino acid sequence for additional refinement or as a drug. The first amino acid sequence can be administered to a subject. A nucleic acid encoding the first amino acid sequence can be administered to a subject. In a related preferred embodiment, the first amino acid sequence is the drug target, and the second amino acid sequence is the drug candidate.

In a preferred embodiment, each first amino acid sequence in the plurality of addresses is unique. For example, a first amino acid sequence can differ from all other test amino acid sequence of the plurality by 1, or more amino acid differences, (e.g., about 2, 3, 4, 5, 8, 16, 32, 64 or more differences; and, by way of example, has about 800, 256, 128, 64, or 32, 16, 8, 4, or fewer differences). In another preferred embodiment, the first amino acid sequence encoded by the nucleic acid at each address of the plurality is identical to all other first amino acid sequences in the plurality of addresses. In a preferred embodiment, the affinity tag encoded by the first nucleic acid at each address of the plurality is the same, or substantially identical to all other affinity tags in the plurality of addresses. In another preferred embodiment, the first nucleic acid at each address of the plurality encodes more than one affinity tag. In yet another preferred embodiment, the affinity tag encoded by the first nucleic acid at an address of the plurality differs from at least one other affinity tag in the plurality of addresses.

In a preferred embodiment, the affinity tag is fused directly to the test amino acid sequence, e.g., directly amino-terminal, or directly carboxy-terminal. In another preferred embodiment, the affinity tag is separated from the test amino acid by one or more linker amino acids, e.g., 1, 2, 3, 4, 5, 6, 8, 10, 12, 20, 30 or more amino acids, preferably about 1 to 20, or about 3 to 12 amino acids. The linker amino acids can include a cleavage site, flexible amino acids (e.g., glycine, alanine, or serine, preferably glycine), and/or polar amino acids. The linker and affinity tag can be amino-terminal or carboxy-terminal to the test amino acid sequence.

The first and/or second nucleic acid can be a RNA, or a DNA (e.g., a single-stranded DNA, or a double stranded DNA). In a preferred embodiment, the first and/or second nucleic acid includes a plasmid DNA or a fragment thereof, an amplification product (e.g., a product generated by RCA, PCR, NASBA); or a synthetic DNA.

The first and/or second nucleic acid can further include one or more of: a transcription promoter; a transcription regulatory sequence; a untranslated leader sequence; a sequence encoding a cleavage site; a recombination site; a 3′ untranslated sequence; a transcriptional terminator; and an internal ribosome entry site. In one embodiment, the nucleic acid sequence includes a plurality of cistrons (also termed “open reading frames”), e.g., the sequence is dicistronic or polycistronic. In another embodiment, the nucleic acid also includes a sequence encoding a reporter protein, e.g., a protein whose abundance can be quantitated and can provide an indication of the quantity of test polypeptide fixed to the plate. The reporter protein can be attached to the test polypeptide, e.g., covalently attached, e.g., attached as a translational fusion. The reporter protein can be an enzyme, e.g., β-galactosidase, chloramphenicol acetyl transferase, β-glucuronidase, and so forth. The reporter protein can produce or modulate light, e.g., a fluorescent protein (e.g., green fluorescent protein, variants thereof, red fluorescent protein, variants thereof, and the like), and luciferase.

The transcription promoter can be a prokaryotic promoter, a eukaryotic promoter, or a viral promoter. In a preferred embodiment, the promoter is the T7 RNA polymerase promoter. The regulatory components, e.g., the transcription promoter, can vary among nucleic acids at different addresses of the plurality. For example, different promoters can be used to vary the amount of polypeptide produced at different addresses.

In one embodiment, the first and/or second nucleic acid also includes at least one site for recombination, e.g., homologous recombination or site-specific recombination, e.g., a lambda att site or variant thereof, a lox site; or a FLP site. In a preferred embodiment, the recombination site lacks stop codons in the reading frame of a nucleic acid encoding a test amino acid sequence. In another preferred embodiment, the recombination site includes a stop codon in the reading frame of a nucleic acid encoding a test amino acid sequence.

In another embodiment, the first and/or second nucleic acid includes a sequence encoding a cleavage site, e.g., a protease site, e.g., a site cleaved by a site-specific protease (e.g., a thrombin site, an enterokinase site, a PreScission site, a factor Xa site, or a TEV site), or a chemical cleavage site (e.g., a methionine, preferably a unique methionine (cleavage by cyanogen bromide) or a proline (cleavage by formic acid)).

The first nucleic acid can include a sequence encoding a second polypeptide tag in addition to the affinity tag. The second tag can be C-terminal to the test amino acid sequence and the affinity tag can be N-terminal to the test amino acid sequence; the second tag can be N-terminal to the test amino acid sequence, and the affinity tag can be C-terminal to the test amino acid sequence; the second tag and the affinity tag can be adjacent to one another, or separated by a linker sequence, both being N-terminal or C-terminal to the test amino acid sequence. In one embodiment, the second tag is an additional affinity tag, e.g., the same or different from the first tag. In another embodiment, the second tag is a recognition tag. For example, the recognition tag can report the presence and/or amount of test polypeptide at an address. Preferably the recognition tag has a sequence other than the sequence of the affinity tag. In still another embodiment, a plurality of polypeptide tags (e.g., less than 3, 4, 5, about 10, or about 20 tags) are encoded in addition to the first affinity tag. Each polypeptide tag of the plurality can be the same as or different from the first affinity tag.

The first and/or second nucleic acid sequence can further include an identifier sequence, e.g., a non-coding nucleic acid sequence, e.g., one that is synthetically inserted, and allows for uniquely identifying the nucleic acid sequence. The identifier sequence can be sufficient in length to uniquely identify each sequence in the plurality; e.g., it is about 5 to 500, 10 to 100, 10 to 50, or about 10 to 30 nucleotides in length. The identifier can be selected so that it is not complementary or identical to another identifier or any region of each nucleic acid sequence of the plurality on the array.

The first and/or second amino acid sequence can further include a protein splicing sequence or intein. The intein can be inserted in the middle of a test amino acid sequence. The intein can be a naturally-occurring intein or a mutated intein.

The first and/or second nucleic acid sequences encoding the first and/or second amino acid sequences can be obtained from a collection of full-length expressed genes (e.g., a repository of clones), a cDNA library, or a genomic library. The first and/or second nucleic acid sequences can be nucleic acids expressed in a tissue, e.g., a normal or diseased tissue. The first and/or second amino acid sequences can be mutants or variants of a scaffold protein (e.g., an antibody, zinc-finger, polypeptide hormone etc.). In yet another embodiment, they are random amino acid sequences, patterned amino acids sequences, or designed amino acids sequences (e.g., sequence designed by manual, rational, or computer-aided approaches).

The binding agent can be attached to the substrate. For example, the substrate can be derivatized and the binding agent covalent attached thereto. The binding agent can be attached via a bridging moiety, e.g., a specific binding pair. (e.g., the substrate contains a first member of a specific binding pair, and the binding agent is linked to the second member of the binding pair, the second member being attached to the substrate). In yet another embodiment, an insoluble substrate (e.g., a bead or particle), is disposed at each address of the plurality, and the binding agent is attached to the insoluble substrate. The insoluble substrate can further contain information encoding its identity, e.g., a reference to the address on which it is disposed. The insoluble substrate can be tagged using a chemical tag, or an electronic tag (e.g., a transponder). The insoluble substrate can be disposed such that it can be removed for later analysis.

In another aspect the invention features a method of identifying an enzyme substrate or cofactor. The method includes: (1) providing a substrate with a plurality of addresses, each address of the plurality comprising (i) a first nucleic acid encoding a hybrid amino acid sequence comprising a first amino acid sequence and an affinity tag, (ii) a binding agent that recognizes the affinity tag and is attached to the substrate, and (iii) a second nucleic acid encoding an enzyme; (2) contacting each address of the plurality with a translation effector to thereby translate the first and second nucleic acid sequences; (3) maintaining the substrate under conditions permissive for the hybrid amino acid sequence to bind binding agent and for activity of the enzyme; (4) detecting the activity of the enzyme at each address of the plurality.

In one embodiment, the first amino acid sequence varies among the addresses of the plurality. In another embodiment, the second nucleic acid varies among the addresses of the plurality.

The method can further include contacting each address of the plurality with an enzyme substrate (e.g., radioactive or otherwise labeled such as with ATP, GTP, s-adenosylmethionine, ubiquitin, and so forth) or a cofactor, e.g., NADH, NADPH, FAD. A substrate or cofactor can be provided with the translation effector.

The detecting step can include monitoring a protein bound by the labeled binding agent (radioactive or otherwise), e.g., after a wash step. The label can be present in solution (e.g., as a cofactor or reaction substrate) and can be transferred to first amino acid sequence by the enzyme, e.g., such that the label is covalently attached to the first amino acid sequence (e.g., such as in phosphorylation). The label can be present in solution and can be bound to the first amino acid sequence (e.g., non-covalently) as a result of an enzyme catalyzed or assisted reaction (e.g., the enzyme can effect a conformational change in the first amino acid sequence, such as a GTP exchange factor protein acting on a GTP binding protein).

In one preferred embodiment, the first amino acid sequence is common to all addresses of the plurality, and a second test amino acid sequence is unique among all the addresses of the plurality. For example, the second test amino acid sequences can be query sequences whereas the first amino test amino acid sequence can be a target sequence. In another preferred embodiment, the first amino acid sequence is unique among all the addresses of the plurality, and the second amino acid sequence is common to all addresses of the plurality. For example, the first test amino acid sequences can be query sequences whereas the second amino test amino acid sequence can be a target sequence. The second nucleic acid encoding the second test amino acid sequence can include a sequence encoding a recognition tag and/or an affinity tag.

In a preferred embodiment, each first amino acid sequence in the plurality of addresses is unique. For example, a first amino acid sequence can differ from all other test amino acid sequence of the plurality by 1, or more amino acid differences, (e.g., about 2, 3, 4, 5, 8, 16, 32, 64 or more differences; and, by way of example, has about 800, 256, 128, 64, or 32, 16, 8, 4, or fewer differences). In another preferred embodiment, the first amino acid sequence encoded by the nucleic acid at each address of the plurality is identical to all other first amino acid sequences in the plurality of addresses. In a preferred embodiment, the affinity tag encoded by the first nucleic acid at each address of the plurality is the same, or substantially identical to all other affinity tags in the plurality of addresses. In another preferred embodiment, the first nucleic acid at each address of the plurality encodes more than one affinity tag. In yet another preferred embodiment, the affinity tag encoded by the first nucleic acid at an address of the plurality differs from at least one other affinity tag in the plurality of addresses.

In a preferred embodiment, the affinity tag is fused directly to the test amino acid sequence, e.g., directly amino-terminal, or directly carboxy-terminal. In another preferred embodiment, the affinity tag is separated from the test amino acid by one or more linker amino acids, e.g., 1, 2, 3, 4, 5, 6, 8, 10, 12, 20, 30 or more amino acids, preferably about 1 to 20, or about 3 to 12 amino acids. The linker amino acids can include a cleavage site, flexible amino acids (e.g., glycine, alanine, or serine, preferably glycine), and/or polar amino acids. The linker and affinity tag can be amino-terminal or carboxy-terminal to the test amino acid sequence.

The first and/or second nucleic acid can be a RNA, or a DNA (e.g., a single-stranded DNA, or a double stranded DNA). In a preferred embodiment, the first and/or second nucleic acid includes a plasmid DNA or a fragment thereof; an amplification product (e.g., a product generated by RCA, PCR, NASBA); or a synthetic DNA.

The first and/or second nucleic acid can further include one or more of: a transcription promoter; a transcription regulatory sequence; a untranslated leader sequence; a sequence encoding a cleavage site; a recombination site; a 3′ untranslated sequence; a transcriptional terminator; and an internal ribosome entry site. In one embodiment, the nucleic acid sequence includes a plurality of cistrons (also termed “open reading frames”), e.g., the sequence is dicistronic or polycistronic. In another embodiment, the nucleic acid also includes a sequence encoding a reporter protein, e.g., a protein whose abundance can be quantitated and can provide an indication of the quantity of test polypeptide fixed to the plate. The reporter protein can be attached to the test polypeptide, e.g., covalently attached, e.g., attached as a translational fusion. The reporter protein can be an enzyme, e.g., β-galactosidase, chloramphenicol acetyl transferase, β-glucuronidase, and so forth. The reporter protein can produce or modulate light, e.g., a fluorescent protein (e.g., green fluorescent protein, variants thereof, red fluorescent protein, variants thereof, and the like), and luciferase.

The transcription promoter can be a prokaryotic promoter, a eukaryotic promoter, or a viral promoter. In a preferred embodiment, the promoter is the T7 RNA polymerase promoter. The regulatory components, e.g., the transcription promoter, can vary among nucleic acids at different addresses of the plurality. For example, different promoters can be used to vary the amount of polypeptide produced at different addresses.

In one embodiment, the first and/or second nucleic acid also includes at least one site for recombination, e.g., homologous recombination or site-specific recombination, e.g., a lambda att site or variant thereof; a lox site; or a FLP site. In a preferred embodiment, the recombination site lacks stop codons in the reading frame of a nucleic acid encoding a test amino acid sequence. In another preferred embodiment, the recombination site includes a stop codon in the reading frame of a nucleic acid encoding a test amino acid sequence.

In another embodiment, the first and/or second nucleic acid includes a sequence encoding a cleavage site, e.g., a protease site, e.g., a site cleaved by a site-specific protease (e.g., a thrombin site, an enterokinase site, a PreScission site, a factor Xa site, or a TEV site), or a chemical cleavage site (e.g., a methionine, preferably a unique methionine (cleavage by cyanogen bromide) or a proline (cleavage by formic acid)).

The first nucleic acid can include a sequence encoding a second polypeptide tag in addition to the affinity tag. The second tag can be C-terminal to the test amino acid sequence and the affinity tag can be N-terminal to the test amino acid sequence; the second tag can be N-terminal to the test amino acid sequence, and the affinity tag can be C-terminal to the test amino acid sequence; the second tag and the affinity tag can be adjacent to one another, or separated by a linker sequence, both being N-terminal or C-terminal to the test amino acid sequence. In one embodiment, the second tag is an additional affinity tag, e.g., the same or different from the first tag. In another embodiment, the second tag is a recognition tag. For example, the recognition tag can report the presence and/or amount of test polypeptide at an address. Preferably the recognition tag has a sequence other than the sequence of the affinity tag. In still another embodiment, a plurality of polypeptide tags (e.g., less than 3, 4, 5, about 10, or about 20 tags) are encoded in addition to the first affinity tag. Each polypeptide tag of the plurality can be the same as or different from the first affinity tag.

The first and/or second nucleic acid sequence can further include an identifier sequence, e.g., a non-coding nucleic acid sequence, e.g., one that is synthetically inserted, and allows for uniquely identifying the nucleic acid sequence. The identifier sequence can be sufficient in length to uniquely identify each sequence in the plurality; e.g., it is about 5 to 500, 10 to 100, 10 to 50, or about 10 to 30 nucleotides in length. The identifier can be selected so that it is not complementary or identical to another identifier or any region of each nucleic acid sequence of the plurality on the array.

The first and/or second amino acid sequence can further include a protein splicing sequence or intein. The intein can be inserted in the middle of a test amino acid sequence. The intein can be a naturally-occurring intein or a mutated intein.

The first and/or second nucleic acid sequences encoding the first and/or second amino acid sequences can be obtained from a collection of full-length expressed genes (e.g., a repository of clones), a cDNA library, or a genomic library. The first and/or second nucleic acid sequences can be nucleic acids expressed in a tissue, e.g., a normal or diseased tissue. The first and/or second amino acid sequences can be mutants or variants of a scaffold protein (e.g., an antibody, zinc-finger, polypeptide hormone etc.). In yet another embodiment, they are random amino acid sequences, patterned amino acids sequences, or designed amino acids sequences (e.g., sequence designed by manual, rational, or computer-aided approaches).

The binding agent can be attached to the substrate. For example, the substrate can be derivatized and the binding agent covalent attached thereto. The binding agent can be attached via a bridging moiety, e.g., a specific binding pair. (e.g., the substrate contains a first member of a specific binding pair, and the binding agent is linked to the second member of the binding pair, the second member being attached to the substrate). In yet another embodiment, an insoluble substrate (e.g., a bead or particle), is disposed at each address of the plurality, and the binding agent is attached to the insoluble substrate. The insoluble substrate can further contain information encoding its identity, e.g., a reference to the address on which it is disposed. The insoluble substrate can be tagged using a chemical tag, or an electronic tag (e.g., a transponder). The insoluble substrate can be disposed such that it can be removed for later analysis.

In another aspect, the invention features a method of producing a protein-interaction map for a plurality of amino acid sequences. The method includes: (1) providing (i) a first plurality of nucleic acid sequences, each encoding an amino acid sequence comprising an amino acid sequence of the plurality of amino acid sequences and an affinity tag; (ii) a second plurality of nucleic acid, each encoding an amino acid sequence comprising an amino acid sequence of the plurality of amino acid sequences and recognition tag; and (iii) a substrate with a plurality of addresses and a binding agent that binds the affinity tag and is attached to the substrate; (2) disposing on the substrate, at each address of the plurality of addresses, a nucleic acid of the first plurality and a nucleic acid of the second plurality; (3) contacting each address of the plurality of addresses with a translation effector to thereby translate the first and second nucleic acid sequences; (4) maintaining the substrate under conditions permissive for the affinity tag to bind binding agent; (5) optionally washing the substrate to remove the translation effector and unbound polypeptides; and (6) detecting the recognition tag at each address of the plurality.

In a preferred embodiment, all possible pairs of amino acid sequences from the plurality of amino acid sequences are present on the array.

Also featured is a database, e.g., in computer memory or a computer readable medium. Each record of the database can include a field for the amino acid sequence encoded by the first nucleic acid sequence, a field for the amino acid sequence encoded by the second nucleic acid sequence, and a field representing the result (e.g., a qualitative or quantitative result) of detecting the recognition tag in the aforementioned method. The database can include a record for each address of the plurality present on the array. Further the database can include a descriptor or reference for the physical location of the nucleic acid sequence on the array. The records can be clustered or have a reference to other records (e.g., including hierarchical groupings) based on the result.

Also featured is a method of providing tagged polypeptides. The method includes: (1) providing a substrate with a plurality of addresses, each address of the plurality comprising (i) a nucleic acid encoding an amino acid sequence comprising a test amino acid sequence and an affinity tag, and (ii) a particle attached to a binding agent that recognizes the affinity tag; (2) contacting each address of the plurality with a translation effector to thereby translate the amino acid sequence; and (3) maintaining the substrate under conditions permissive for the amino acid sequence to contact the binding agent.

In one preferred embodiment, the nucleic acid sequence is also attached to the particle.

In another preferred embodiment, the particle, e.g., a bead or nanoparticle, further contains information encoding its identity, e.g., a reference to the address on which it is disposed. The particle can be tagged using a chemical tag, or an electronic tag (e.g., a transponder). The particles can be disposed on the substrate such that they can be removed for later analysis. In one embodiment, multiple particles with the same identifier are disposed at each address of the plurality. The particles can be collected after translation and attachment of the amino acid sequence. The particles can then be subdivided into aliquots. A particle with a given property, e.g., the ability to bind a labeled compound can be identified. The identity of the particle can be determined to thereby identify the amino acid sequence attached to the particle.

In a preferred embodiment, each test amino acid sequence in the plurality of addresses is unique. For example, a test amino acid sequence can differ from all other test amino acid sequence of the plurality by 1, or more amino acid differences, (e.g., about 2, 3, 4, 5, 8, 16, 32, 64 or more differences; and, by way of example, has about 800, 256, 128, 64, or 32, 16, 8, 4, or fewer differences). In another preferred embodiment, the test amino acid sequence encoded by the nucleic acid at each address of the plurality is identical to all other test amino acid sequences in the plurality of addresses. In a preferred embodiment, the affinity tag encoded by the nucleic acid at each address of the plurality is the same, or substantially identical to all other affinity tags in the plurality of addresses. In another preferred embodiment, the nucleic acid at each address of the plurality encodes more than one affinity tag. In yet another preferred embodiment, the affinity tag encoded by the nucleic acid at an address of the plurality differs from at least one other affinity tag in the plurality of addresses.

In a preferred embodiment, the affinity tag is fused directly to the test amino acid sequence, e.g., directly amino-terminal, or directly carboxy-terminal. In another preferred embodiment, the affinity tag is separated from the test amino acid by one or more linker amino acids, e.g., 1, 2, 3, 4, 5, 6, 8, 10, 12, 20, 30 or more amino acids, preferably about 1 to 20, or about 3 to 12 amino acids. The linker amino acids can include a cleavage site, flexible amino acids (e.g., glycine, alanine, or serine, preferably glycine), and/or polar amino acids. The linker and affinity tag can be amino-terminal or carboxy-terminal to the test amino acid sequence.

The nucleic acid can be a RNA, or a DNA (e.g., a single-stranded DNA, or a double stranded DNA). In a preferred embodiment, the nucleic acid includes a plasmid DNA or a fragment thereof; an amplification product (e.g., a product generated by RCA, PCR, NASBA); or a synthetic DNA.

The nucleic acid can further include one or more of: a transcription promoter; a transcription regulatory sequence; a untranslated leader sequence; a sequence encoding a cleavage site; a recombination site; a 3′ untranslated sequence; a transcriptional terminator; and an internal ribosome entry site. In one embodiment, the nucleic acid sequence includes a plurality of cistrons (also termed “open reading frames”), e.g., the sequence is dicistronic or polycistronic. In another embodiment, the nucleic acid also includes a sequence encoding a reporter protein, e.g., a protein whose abundance can be quantitated and can provide an indication of the quantity of test polypeptide fixed to the plate. The reporter protein can be attached to the test polypeptide, e.g., covalently attached, e.g., attached as a translational fusion. The reporter protein can be an enzyme, e.g., β-galactosidase, chloramphenicol acetyl transferase, β-glucuronidase, and so forth. The reporter protein can produce or modulate light, e.g., a fluorescent protein (e.g., green fluorescent protein, variants thereof, red fluorescent protein, variants thereof, and the like), and luciferase.

The transcription promoter can be a prokaryotic promoter, a eukaryotic promoter, or a viral promoter. In a preferred embodiment, the promoter is the T7 RNA polymerase promoter. The regulatory components, e.g., the transcription promoter, can vary among nucleic acids at different addresses of the plurality. For example, different promoters can be used to vary the amount of polypeptide produced at different addresses.

In one embodiment, the nucleic acid also includes at least one site for recombination, e.g., homologous recombination or site-specific recombination, e.g., a lambda att site or variant thereof; a lox site; or a FLP site. In a preferred embodiment, the recombination site lacks stop codons in the reading frame of a nucleic acid encoding a test amino acid sequence. In another preferred embodiment, the recombination site includes a stop codon in the reading frame of a nucleic acid encoding a test amino acid sequence.

In another embodiment, the nucleic acid includes a sequence encoding a cleavage site, e.g., a protease site, e.g., a site cleaved by a site-specific protease (e.g., a thrombin site, an enterokinase site, a PreScission site, a factor Xa site, or a TEV site), or a chemical cleavage site (e.g., a methionine, preferably a unique methionine (cleavage by cyanogen bromide) or a proline (cleavage by formic acid)).

The nucleic acid can include a sequence encoding a second polypeptide tag in addition to the affinity tag. The second tag can be C-terminal to the test amino acid sequence and the affinity tag can be N-terminal to the test amino acid sequence; the second tag can be N-terminal to the test amino acid sequence, and the affinity tag can be C-terminal to the test amino acid sequence; the second tag and the affinity tag can be adjacent to one another, or separated by a linker sequence, both being N-terminal or C-terminal to the test amino acid sequence. In one embodiment, the second tag is an additional affinity tag, e.g., the same or different from the first tag. In another embodiment, the second tag is a recognition tag. For example, the recognition tag can report the presence and/or amount of test polypeptide at an address. Preferably the recognition tag has a sequence other than the sequence of the affinity tag. In still another embodiment, a plurality of polypeptide tags (e.g., less than 3, 4, 5, about 10, or about 20 tags) are encoded in addition to the first affinity tag. Each polypeptide tag of the plurality can be the same as or different from the first affinity tag.

The nucleic acid sequence can further include an identifier sequence, e.g., a non-coding nucleic acid sequence, e.g., one that is synthetically inserted, and allows for uniquely identifying the nucleic acid sequence. The identifier sequence can be sufficient in length to uniquely identify each sequence in the plurality; e.g., it is about 5 to 500, 10 to 100, 10 to 50, or about 10 to 30 nucleotides in length. The identifier can be selected so that it is not complementary or identical to another identifier or any region of each nucleic acid sequence of the plurality on the array.

The test amino acid sequence can further include a protein splicing sequence or intein. The intein can be inserted in the middle of a test amino acid sequence. The intein can be a naturally-occurring intein or a mutated intein.

The nucleic acid sequences encoding the test amino acid sequences can be obtained from a collection of full-length expressed genes (e.g., a repository of clones), a cDNA library, or a genomic library. The test amino acid sequences can be genes expressed in a tissue, e.g., a normal or diseased tissue. The test polypeptides can be mutants or variants of a scaffold protein (e.g., an antibody, zinc-finger, polypeptide hormone etc.). In yet another embodiment, the test polypeptides are random amino acid sequences, patterned amino acids sequences, or designed amino acids sequences (e.g., sequence designed by manual, rational, or computer-aided approaches). The plurality of test amino acid sequences can include a plurality from a first source, and plurality from a second source. For example, the test amino acid sequences on half the addresses of an array are from a diseased tissue or a first species, whereas the sequences on the remaining half are from a normal tissue or a second species.

The binding agent can be attached to the substrate. For example, the substrate can be derivatized and the binding agent covalent attached thereto. The binding agent can be attached via a bridging moiety, e.g., a specific binding pair. (e.g., the substrate contains a first member of a specific binding pair, and the binding agent is linked to the second member of the binding pair, the second member being attached to the substrate).

In another aspect, the invention features a method of providing tagged polypeptides. The method includes: providing a substrate with a plurality of addresses, each address of the plurality having a nucleic acid (i) encoding an amino acid sequence comprising: (1) a test amino acid sequence, and (2) a tag; and (ii) a handle; contacting each address of the plurality with a translation effector to thereby translate the nucleic acid sequence; and maintaining the substrate under conditions permissive for the tag to contact the handle to thereby form a complex of the nucleic acid and the test polypeptide having the test amino acid sequence.

In one embodiment, the handle is biotin, and the tag is avidin. For example, the nucleic acid has a biotin covalent attached to a nucleotide. The nucleic acid can be formed by amplification of a template nucleic acid using a synthetic oligonucleotide having a biotin moiety covalently attached at its 5′ end. In another embodiment, the handle is glutathione, and the tag is glutathione-S-transferase. For example, the nucleic acid has a glutathione moiety covalent attached to a nucleotide. The nucleic acid can be formed by amplification of a template nucleic acid using a synthetic oligonucleotide having a biotin moiety covalently attached at its 5′ end.

In one embodiment, the handle includes a keto group, and the tag is a hydrazine. A covalent bond is formed between the handle and tag.

The method can further includes combining the complexes formed at all the addresses into a pool, selecting a polypeptide from the pool, and amplifying the complexed nucleic acid sequence to thereby identify the selected amino acid sequence.

In a preferred embodiment, each test amino acid sequence in the plurality of addresses is unique. For example, a test amino acid sequence can differ from all other test amino acid sequence of the plurality by 1, or more amino acid differences, (e.g., about 2, 3, 4, 5, 8, 16, 32, 64 or more differences; and, by way of example, has about 800, 256, 128, 64, or 32, 16, 8, 4, or fewer differences). In another preferred embodiment, the test amino acid sequence encoded by the nucleic acid at each address of the plurality is identical to all other test amino acid sequences in the plurality of addresses. In a preferred embodiment, the affinity tag encoded by the nucleic acid at each address of the plurality is the same, or substantially identical to all other affinity tags in the plurality of addresses. In another preferred embodiment, the nucleic acid at each address of the plurality encodes more than one affinity tag. In yet another preferred embodiment, the affinity tag encoded by the nucleic acid at an address of the plurality differs from at least one other affinity tag in the plurality of addresses.

In a preferred embodiment, the tag is fused directly to the test amino acid sequence, e.g., directly amino-terminal, or directly carboxy-terminal. In another preferred embodiment, the tag is separated from the test amino acid by one or more linker amino acids, e.g., 1, 2, 3, 4, 5, 6, 8, 10, 12, 20, 30 or more amino acids, preferably about 1 to 20, or about 3 to 12 amino acids. The linker amino acids can include a cleavage site, flexible amino acids (e.g., glycine, alanine, or serine, preferably glycine), and/or polar amino acids. The linker and tag can be amino-terminal or carboxy-terminal to the test amino acid sequence.

The nucleic acid can be an RNA, or a DNA (e.g., a single-stranded DNA, or a double stranded DNA). In a preferred embodiment, the nucleic acid includes a plasmid DNA or a fragment thereof; an amplification product (e.g., a product generated by RCA, PCR, NASBA); or a synthetic DNA.

The nucleic acid can further include one or more of: a transcription promoter; a transcription regulatory sequence; a untranslated leader sequence; a sequence encoding a cleavage site; a recombination site; a 3′ untranslated sequence; a transcriptional terminator; and an internal ribosome entry site. In one embodiment, the nucleic acid sequence includes a plurality of cistrons (also termed “open reading frames”), e.g., the sequence is dicistronic or polycistronic. In another embodiment, the nucleic acid also includes a sequence encoding a reporter protein, e.g., a protein whose abundance can be quantitated and can provide an indication of the quantity of test polypeptide fixed to the plate. The reporter protein can be attached to the test polypeptide, e.g., covalently attached, e.g., attached as a translational fusion. The reporter protein can be an enzyme, e.g., β-galactosidase, chloramphenicol acetyl transferase, β-glucuronidase, and so forth. The reporter protein can produce or modulate light, e.g., a fluorescent protein (e.g., green fluorescent protein, variants thereof, red fluorescent protein, variants thereof, and the like), and luciferase.

The transcription promoter can be a prokaryotic promoter, a eukaryotic promoter, or a viral promoter. In a preferred embodiment, the promoter is the T7 RNA polymerase promoter. The regulatory components, e.g., the transcription promoter, can vary among nucleic acids at different addresses of the plurality. For example, different promoters can be used to vary the amount of polypeptide produced at different addresses.

In one embodiment, the nucleic acid also includes at least one site for recombination, e.g., homologous recombination or site-specific recombination, e.g., a lambda att site or variant thereof; a lox site; or a FLP site. In a preferred embodiment, the recombination site lacks stop codons in the reading frame of a nucleic acid encoding a test amino acid sequence. In another preferred embodiment, the recombination site includes a stop codon in the reading frame of a nucleic acid encoding a test amino acid sequence.

In another embodiment, the nucleic acid includes a sequence encoding a cleavage site, e.g., a protease site, e.g., a site cleaved by a site-specific protease (e.g., a thrombin site, an enterokinase site, a PreScission site, a factor Xa site, or a TEV site), or a chemical cleavage site (e.g., a methionine, preferably a unique methionine (cleavage by cyanogen bromide) or a proline (cleavage by formic acid)).

The nucleic acid can include a sequence encoding a second polypeptide tag in addition to the first tag. The second tag can be C-terminal to the test amino acid sequence and the first tag can be N-terminal to the test amino acid sequence; the second tag can be N-terminal to the test amino acid sequence, and the first tag can be C-terminal to the test amino acid sequence; the second tag and the first tag can be adjacent to one another, or separated by a linker sequence, both being N-terminal or C-terminal to the test amino acid sequence. In one embodiment, the second tag is an additional affinity tag, e.g., the same or different from the first tag. In another embodiment, the second tag is a recognition tag. For example, the recognition tag can report the presence and/or amount of test polypeptide at an address. Preferably the recognition tag has a sequence other than the sequence of the affinity tag. In still another embodiment, a plurality of polypeptide tags (e.g., less than 3, 4, 5, about 10, or about 20 tags) are encoded in addition to the first affinity tag. Each polypeptide tag of the plurality can be the same as or different from the first tag.

The nucleic acid sequence can further include an identifier sequence, e.g., a non-coding nucleic acid sequence, e.g., one that is synthetically inserted, and allows for uniquely identifying the nucleic acid sequence. The identifier sequence can be sufficient in length to uniquely identify each sequence in the plurality; e.g., it is about 5 to 500, 10 to 100, 10 to 50, or about 10 to 30 nucleotides in length. The identifier can be selected so that it is not complementary or identical to another identifier or any region of each nucleic acid sequence of the plurality on the array.

The test amino acid sequence can further include a protein splicing sequence or intein. The intein can be inserted in the middle of a test amino acid sequence. The intein can be a naturally-occurring intein or a mutated intein.

The nucleic acid sequences encoding the test amino acid sequences can be obtained from a collection of full-length expressed genes (e.g., a repository of clones), a cDNA library, or a genomic library. The test amino acid sequences can be genes expressed in a tissue, e.g., a normal or diseased tissue. The test polypeptides can be mutants or variants of a scaffold protein (e.g., an antibody, zinc-finger, polypeptide hormone etc.). In yet another embodiment, the test polypeptides are random amino acid sequences, patterned amino acids sequences, or designed amino acids sequences (e.g., sequence designed by manual, rational, or computer-aided approaches). The plurality of test amino acid sequences can include a plurality from a first source, and plurality from a second source. For example, the test amino acid sequences on half the addresses of an array are from a diseased tissue or a first species, whereas the sequences on the remaining half are from a normal tissue or a second species.

The handle can be attached to the substrate. For example, the substrate can be derivatized and the handle covalent attached thereto. The handle can be attached via a bridging moiety, e.g., a specific binding pair. (e.g., the substrate contains a first member of a specific binding pair, and the handle is linked to the second member of the binding pair, the second member being attached to the substrate).

In yet another embodiment, an insoluble substrate (e.g., a bead or particle), is disposed at each address of the plurality, and the handle is attached to the insoluble substrate. The insoluble substrate can further contain information encoding its identity, e.g., a reference to the address on which it is disposed. The insoluble substrate can be tagged using a chemical tag, or an electronic tag (e.g., a transponder). The insoluble substrate can be disposed such that it can be removed for later analysis.

The invention also features a kit which includes: (1) an array comprising a plurality of addresses, wherein each address of the plurality comprises a handle and (2) a vector nucleic acid comprising (i) a promoter; (ii) an entry site; and (iii) a tag encoding sequence, wherein the tag can be attached to the handle.

The vector nucleic acid can include one or more sites for insertion of a test amino acid sequence (e.g., a recombination site or a restriction site), and a sequence encoding an tag. In a preferred embodiment, the vector nucleic acid has two sites for insertion, and a toxic gene inserted between the two sites. In another embodiment, the sites for insertion are homologous recombination or site-specific recombination sites, e.g., a lambda att site or variant thereof; a lox site; or a FLP site. In a preferred embodiment, one or both recombination sites lack stop codons in the reading frame of a nucleic acid encoding a test amino acid sequence. In another preferred embodiment, one or both recombination sites include a stop codon in the reading frame of a nucleic acid encoding a test amino acid sequence.

In a much preferred embodiment, the tag is in frame with the translation frame of a nucleic acid sequence (e.g., a sequence to be inserted) encoding a test amino acid sequence. In a preferred embodiment, the tag is fused directly to the test amino acid sequence, e.g., directly amino-terminal, or directly carboxy-terminal. In another preferred embodiment, the tag is separated from the test amino acid by one or more linker amino acids, e.g., 1, 2, 3, 4, 5, 6, 8, 10, 12, 20, 30 or more amino acids, preferably about 1 to 20, or about 3 to 12 amino acids. The linker amino acids can include a cleavage site, flexible amino acids (e.g., glycine, alanine, or serine, preferably glycine), and/or polar amino acids. The linker and tag can be amino-terminal or carboxy-terminal to the test amino acid sequence. The cleavage site can be a protease site, e.g., a site cleaved by a site-specific protease (e.g., a thrombin site, an enterokinase site, a PreScission site, a factor Xa site, or a TEV site), or a chemical cleavage site (e.g., a methionine, preferably a unique methionine (cleavage by cyanogen bromide) or a proline (cleavage by formic acid)).

In one embodiment, the handle includes a keto group, and the tag is a hydrazine. A covalent bond is formed between the handle and tag. The kit can further include an unnatural amino acid having a keto group, e.g., a reactable keto group on a side chain. The kit can also further include a tRNA, and optionally a tRNA synthetase for amino-acylating the tRNA with the unnatural amino acid. The tRNA can be a stop codon suppressing tRNA.

In a preferred embodiment, the kit also includes at least a second vector nucleic acid. The second vector nucleic acid can include one or more sites for insertion of a test amino acid sequence (e.g., a recombination site or a restriction site).

In another embodiment, the kit also includes multiple nucleic acids encoding unique test amino acid sequences. These encoding nucleic acids can be flanked, e.g., on both ends by a site, e.g., a site compatible with the vector nucleic acid (e.g., having sequence for recombination with a sequence in the vector; or having a restriction site which leaves an overhang or blunt end such that the overhang or blunt end can be ligated into the vector nucleic acid (e.g., the restricted vector nucleic acid)).

In another preferred embodiment, the kit also includes a transcription effector and/or a translation effector.

In a preferred embodiment, the second vector nucleic acid has a recognition tag, e.g., an epitope tag, an enzyme, a fluorescent protein (e.g., GFP, BFP, variants thereof).

The first and/or second vector nucleic acid can further include one or more of: a transcription promoter; a transcription regulatory sequence; a untranslated leader sequence; a sequence encoding a cleavage site; a recombination site; a 3′ untranslated sequence; a transcriptional terminator; and an internal ribosome entry site. In one embodiment, the nucleic acid sequence includes a plurality of cistrons (also termed “open reading frames”), e.g., the sequence is dicistronic or polycistronic. In another embodiment, the nucleic acid also includes a sequence encoding a reporter protein, e.g., a protein whose abundance can be quantitated and can provide an indication of the quantity of test polypeptide fixed to the plate. The reporter protein can be attached to the test polypeptide, e.g., covalently attached, e.g., attached as a translational fusion. The reporter protein can be an enzyme, e.g., β-galactosidase, chloramphenicol acetyl transferase, β-glucuronidase, and so forth. The reporter protein can produce or modulate light, e.g., a fluorescent protein (e.g., green fluorescent protein, variants thereof, red fluorescent protein, variants thereof, and the like), and luciferase.

The transcription promoter can be a prokaryotic promoter, a eukaryotic promoter, or a viral promoter. In a preferred embodiment, the promoter is the T7 RNA polymerase promoter.

In a preferred embodiment, the kit also includes a recombinase, a ligase, and/or a restriction endonuclease. For example, the recombinase can mediate recombination, e.g., site-specific recombination or homologous recombination, between a recombination site on the test nucleic acid and a recombination sequence on the vector nucleic acid. For example, the recombinase can be lambda integrase, HIV integrase, Cre, or FLP recombinase.

In a preferred embodiment, each address of the plurality has a handle capable of recognizing the tag. The handle can be attached to the substrate. For example, the substrate can be derivatized and the handle covalent attached thereto. The handle can be attached via a bridging moiety, e.g., a specific binding pair. (e.g., the substrate contains a first member of a specific binding pair, and the handle is linked to the second member of the binding pair, the second member being attached to the substrate).

In yet another embodiment, the array of the kit includes an insoluble substrate (e.g., a bead or particle), disposed at each address of the plurality, and the handle is attached to the insoluble substrate. The insoluble substrate can further contain information encoding its identity, e.g., a reference to the address on which it is disposed. The insoluble substrate can be tagged using a chemical tag, or an electronic tag (e.g., a transponder). The insoluble substrate can be disposed such that it can be removed for later analysis.

The first or second vector nucleic acid can include a sequence encoding a second polypeptide tag in addition to the tag. The second tag can be C-terminal to the test amino acid sequence and the tag can be N-terminal to the test amino acid sequence; the second tag can be N-terminal to the test amino acid sequence, and the tag can be C-terminal to the test amino acid sequence; the second tag and the tag can be adjacent to one another, or separated by a linker sequence, both being N-terminal or C-terminal to the test amino acid sequence. In one embodiment, the second tag is an additional tag, e.g., the same or different from the first tag. In another embodiment, the second tag is a recognition tag. For example, the recognition tag can report the presence and/or amount of test polypeptide at an address. Preferably the recognition tag has a sequence other than the sequence of the tag. In still another embodiment, a plurality of polypeptide tags (e.g., less than 3, 4, 5, about 10, or about 20 tags) are encoded in addition to the first tag. Each polypeptide tag of the plurality can be the same as or different from the first tag.

The first or second vector nucleic acid sequence can further include a sequence encoding a protein splicing sequence or intein. The intein can be inserted in the middle of a test amino acid sequence. The intein can be a naturally-occurring intein or a mutated intein.

The nucleic acids encoding the test amino acid sequences can be obtained from a collection of full-length expressed genes (e.g., a repository of clones), a cDNA library, or a genomic library. The encoding nucleic acids can be nucleic acids (e.g., an mRNA or cDNA) expressed in a tissue, e.g., a normal or diseased tissue. The test polypeptides (i.e., test amino acid sequences) can be mutants or variants of a scaffold protein (e.g., an antibody, zinc-finger, polypeptide hormone etc.). In yet another embodiment, the test polypeptides are random amino acid sequences, patterned amino acids sequences, or designed amino acids sequences (e.g., sequence designed by manual, rational, or computer-aided approaches). The plurality of test amino acid sequences can include a plurality from a first source, and plurality from a second source. For example, the test amino acid sequences on half the addresses of an array are from a diseased tissue or a first species, whereas the sequences on the remaining half are from a normal tissue or a second species.

The kit can further include software and/or a database, e.g., in computer memory or a computer readable medium (e.g., a CD-ROM, a magnetic disc, flash memory. Each record of the database can include a field for the test amino acid sequence encoded by the nucleic acid sequence and a descriptor or reference for the physical location of the encoding nucleic acid sequence in the kit, e.g., location in a microtitre plate. Optionally, the record also includes a field representing a result (e.g., a qualitative or quantitative result) of detecting the polypeptide encoded by the nucleic acid sequence. The database can include a record for each address of the plurality present on the array. The records can be clustered or have a reference to other records (e.g., including hierarchical groupings) based on the result. The software can contain computer readable code to configure a computer-controlled robotic apparatus to manipulate nucleic acids encoding test amino acid sequences and vector nucleic acids in order to insert the encoding nucleic acids into the vector nucleic acids and further to manipulate the insertion products onto addresses of the array.

The kit can also include instructions for use of the array or a link or indication of a network resource (e.g., a web site) having instructions for use of the array or the above database of records describing the addresses of the array.

A method of providing an array includes providing the aforementioned kit, and a plurality of nucleic acid sequences, each encoding a unique test amino acid sequence and an excision site. The method further includes removing each of the plurality of nucleic acid sequence from the excision site and inserting it into the entry site of the vector nucleic acid to thereby generate a test nucleic acid sequence encoding a test polypeptide comprising the test amino acid sequence and the tag; and disposing each of the plurality of test nucleic acid sequences at an address of the array.

Another featured kit includes: an array comprising a substrate having a plurality of addresses, wherein each address of the plurality comprises a handle, and a nucleic acid sequence encoding an amino acid sequence comprising: (a) a test amino acid sequence, and (b) a tag. The kit can optionally further include at least one of: a translation effector and a transcription effector.

The nucleic acid can be a RNA, or a DNA (e.g., a single-stranded DNA, or a double stranded DNA). In a preferred embodiment, the nucleic acid includes a plasmid DNA or a fragment thereof; an amplification product (e.g., a product generated by RCA, PCR, NASBA); or a synthetic DNA.

The nucleic acid can further include one or more of: a transcription promoter; a transcription regulatory sequence; a untranslated leader sequence; a sequence encoding a cleavage site; a recombination site; a 3′ untranslated sequence; a transcriptional terminator; and an internal ribosome entry site. In one embodiment, the nucleic acid sequence includes a plurality of cistrons (also termed “open reading frames”), e.g., the sequence is dicistronic or polycistronic. In another embodiment, the nucleic acid also includes a sequence encoding a reporter protein, e.g., a protein whose abundance can be quantitated and can provide an indication of the quantity of test polypeptide fixed to the plate. The reporter protein can be attached to the test polypeptide, e.g., covalently attached, e.g., attached as a translational fusion. The reporter protein can be an enzyme, e.g., β-galactosidase, chloramphenicol acetyl transferase, β-glucuronidase, and so forth. The reporter protein can produce or modulate light, e.g., a fluorescent protein (e.g., green fluorescent protein, variants thereof, red fluorescent protein, variants thereof, and the like), and luciferase.

The transcription promoter can be a prokaryotic promoter, a eukaryotic promoter, or a viral promoter. In a preferred embodiment, the promoter is the T7 RNA polymerase promoter. The regulatory components, e.g., the transcription promoter, can vary among nucleic acids at different addresses of the plurality. For example, different promoters can be used to vary the amount of polypeptide produced at different addresses.

In one embodiment, the nucleic acid also includes at least one site for recombination, e.g., homologous recombination or site-specific recombination, e.g., a lambda att site or variant thereof; a lox site; or a FLP site. In a preferred embodiment, the recombination site lacks stop codons in the reading frame of a nucleic acid encoding a test amino acid sequence. In another preferred embodiment, the recombination site includes a stop codon in the reading frame of a nucleic acid encoding a test amino acid sequence.

In another embodiment, the nucleic acid includes a sequence encoding a cleavage site, e.g., a protease site, e.g., a site cleaved by a site-specific protease (e.g., a thrombin site, an enterokinase site, a PreScission site, a factor Xa site, or a TEV site), or a chemical cleavage site (e.g., a methionine, preferably a unique methionine (cleavage by cyanogen bromide) or a proline (cleavage by formic acid)).

In a preferred embodiment, each test amino acid sequence in the plurality of addresses is unique. For example, a test amino acid sequence can differ from all other test amino acid sequence of the plurality by 1, or more amino acid differences, (e.g., about 2, 3, 4, 5, 8, 16, 32, 64 or more differences; and, by way of example, has about 800, 256, 128, 64, or 32, 16, 8, 4, or fewer differences). In another preferred embodiment, the test amino acid sequence encoded by the nucleic acid at each address of the plurality is identical to all other test amino acid sequences in the plurality of addresses. In a preferred embodiment, the affinity tag encoded by the nucleic acid at each address of the plurality is the same, or substantially identical to all other affinity tags in the plurality of addresses. In another preferred embodiment, the nucleic acid at each address of the plurality encodes more than one affinity tag. In yet another preferred embodiment, the affinity tag encoded by the nucleic acid at an address of the plurality differs from at least one other affinity tag in the plurality of addresses.

In a preferred embodiment, the affinity tag is fused directly to the test amino acid sequence, e.g., directly amino-terminal, or directly carboxy-terminal. In another preferred embodiment, the affinity tag is separated from the test amino acid by one or more linker amino acids, e.g., 1, 2, 3, 4, 5, 6, 8, 10, 12, 20, 30 or more amino acids, preferably about 1 to 20, or about 3 to 12 amino acids. The linker amino acids can include a cleavage site, flexible amino acids (e.g., glycine, alanine, or serine, preferably glycine), and/or polar amino acids. The linker and affinity tag can be amino-terminal or carboxy-terminal to the test amino acid sequence.

The nucleic acid can include a sequence encoding a second polypeptide tag in addition to the affinity tag. The second tag can be C-terminal to the test amino acid sequence and the affinity tag can be N-terminal to the test amino acid sequence; the second tag can be N-terminal to the test amino acid sequence, and the affinity tag can be C-terminal to the test amino acid sequence; the second tag and the affinity tag can be adjacent to one another, or separated by a linker sequence, both being N-terminal or C-terminal to the test amino acid sequence. In one embodiment, the second tag is an additional affinity tag, e.g., the same or different from the first tag. In another embodiment, the second tag is a recognition tag. For example, the recognition tag can report the presence and/or amount of test polypeptide at an address. Preferably the recognition tag has a sequence other than the sequence of the affinity tag. In still another embodiment, a plurality of polypeptide tags (e.g., less than 3, 4, 5, about 10, or about 20 tags) are encoded in addition to the first affinity tag. Each polypeptide tag of the plurality can be the same as or different from the first affinity tag.

The nucleic acid sequence can further include an identifier sequence, e.g., a non-coding nucleic acid sequence, e.g., one that is synthetically inserted, and allows for uniquely identifying the nucleic acid sequence. The identifier sequence can be sufficient in length to uniquely identify each sequence in the plurality; e.g., it is about 5 to 500, 10 to 100, 10 to 50, or about 10 to 30 nucleotides in length. The identifier can be selected so that it is not complementary or identical to another identifier or any region of each nucleic acid sequence of the plurality on the array.

The nucleic acid sequence can further include a sequence encoding a protein splicing sequence or intein. The intein can be inserted in the middle of a test amino acid sequence. The intein can be a naturally-occurring intein or a mutated intein.

The nucleic acids encoding the test amino acid sequences can be obtained from a collection of full-length expressed genes (e.g., a repository of clones), a cDNA library, or a genomic library. The encoding nucleic acids can be nucleic acids (e.g., an mRNA or cDNA) expressed in a tissue, e.g., a normal or diseased tissue. The test polypeptides (i.e., test amino acid sequences) can be mutants or variants of a scaffold protein (e.g., an antibody, zinc-finger, polypeptide hormone etc.). In yet another embodiment, the test polypeptides are random amino acid sequences, patterned amino acids sequences, or designed amino acids sequences (e.g., sequence designed by manual, rational, or computer-aided approaches). The plurality of test amino acid sequences can include a plurality from a first source, and plurality from a second source. For example, the test amino acid sequences on half the addresses of an array are from a diseased tissue or a first species, whereas the sequences on the remaining half are from a normal tissue or a second species.

In a preferred embodiment, each address of the plurality further includes one or more second nucleic acids, e.g., a plurality of unique nucleic acids. Hence, the plurality in toto can encode a plurality of test sequences. For example, each address of the plurality can encode a pool of test polypeptide sequences, e.g., a subset of a library or clone bank. A second array can be provided in which each address of the plurality of the second array includes a single or subset of members of the pool present at an address of the first array. The first and the second array can be used consecutively.

In other preferred embodiments, each address of the plurality further includes a second nucleic acid encoding a second amino acid sequence.

In one preferred embodiment, each address of the plurality includes a first test amino acid sequence that is common to all addresses of the plurality, and a second test amino acid sequence that is unique among all the addresses of the plurality. For example, the second test amino acid sequences can be query sequences whereas the first amino test amino acid sequence can be a target sequence. In another preferred embodiment, each address of the plurality includes a first test amino acid sequence that is unique among all the addresses of the plurality, and a second test amino acid sequence that is common to all addresses of the plurality. For example, the first test amino acid sequences can be query sequences whereas the second amino test amino acid sequence can be a target sequence. The second nucleic acid encoding the second test amino acid sequence can include a sequence encoding a recognition tag and/or an affinity tag.

At at least one address of the plurality, the first and second amino acid sequences can be such that they interact with one another. In one preferred embodiment, they are capable of binding to each other. The second test amino acid sequence is optionally fused to a detectable amino acid sequence, e.g., an epitope tag, an enzyme, a fluorescent protein (e.g., GFP, BFP, variants thereof). The second test amino acid sequence can be itself detectable (e.g., an antibody is available which specifically recognizes it). In another preferred embodiment, one is capable of modifying the other (e.g., making or breaking a bond, preferably a covalent bond, of the other). For example, the first amino acid sequence is kinase capable of phosphorylating the second amino acid sequence; the first is a methylase capable of methylating the second; the first is a ubiquitin ligase capable of ubiquitinating the second; the first is a protease capable of cleaving the second; and so forth.

Kits of these embodiments can be used to identify an interaction or to identify a compound that modulates, e.g., inhibits or enhances, an interaction.

The binding agent can be attached to the substrate. For example, the substrate can be derivatized and the binding agent covalent attached thereto. The binding agent can be attached via a bridging moiety, e.g., a specific binding pair. (e.g., the substrate contains a first member of a specific binding pair, and the binding agent is linked to the second member of the binding pair, the second member being attached to the substrate).

In yet another embodiment, an insoluble substrate (e.g., a bead or particle), is disposed at each address of the plurality, and the binding agent is attached to the insoluble substrate. The insoluble substrate can further contain information encoding its identity, e.g., a reference to the address on which it is disposed. The insoluble substrate can be tagged using a chemical tag, or an electronic tag (e.g., a transponder). The insoluble substrate can be disposed such that it can be removed for later analysis.

The kit can further include a database, e.g., in computer memory or a computer readable medium (e.g., a CD-ROM, a magnetic disc, flash memory. Each record of the database can include a field for the amino acid sequence encoded by the nucleic acid sequence and a descriptor or reference for the physical location of the nucleic acid sequence on the array. Optionally, the record also includes a field representing a result (e.g., a qualitative or quantitative result) of detecting the polypeptide encoded by the nucleic acid sequence. The database can include a record for each address of the plurality present on the array. The records can be clustered or have a reference to other records (e.g., including hierarchical groupings) based on the result.

The kit can also include instructions for use of the array or a link or indication of a network resource (e.g., a web site) having instructions for use of the array or the above database of records describing the addresses of the array.

In another aspect, the invention features a method of providing an array across a network, e.g., a computer network, or a telecommunications network. The method includes: providing a substrate comprising a plurality of addresses, each address of the plurality having a binding agent; providing a plurality of nucleic acid sequences, each nucleic acid sequence comprising a sequence encoding a test amino acid sequence and an affinity tag that is recognized by the binding agent; providing on a server a list of either (i) nucleic acid sequences of the plurality or (ii) subsets of the plurality (e.g., categorized groups of sequences); transmitting the list across a network to a user; receiving at least one selection of the list from the user; disposing the one or more nucleic acid sequence corresponding to the selection on an address of the plurality; and providing the substrate to the user.

In one embodiment, each nucleic acid sequence is disposed at a unique address. For example, if a subset is selected, each nucleic acid sequence of the subset is disposed at a unique address. In another embodiment, a plurality of nucleic acid sequences are disposed at each address.

The method can further include contacting each address of the plurality with one or more of (i) a transcription effector, and (ii) a translation effector. Optionally, the substrate is maintained under conditions permissive for the amino acid sequence to bind the binding agent. One or more addresses can then be washed, e.g., to remove at least one of (i) the nucleic acid, (ii) the transcription effector, (iii) the translation effector, and/or (iv) an unwanted polypeptide, e.g., an unbound polypeptide or unfolded polypeptide. The array can optionally be contacted with a compound, e.g., a chaperone; a protease; a protein-modifying enzyme; a small molecule, e.g., a small organic compound (e.g., of molecular weight less than 5000, 3000, 1000, 700, 500, or 300 Daltons); nucleic acids; or other complex macromolecules e.g., complex sugars, lipids, or matrix molecules.

The array can be further processed, e.g., prepared for storage. It can be enclosed in a package, e.g., an air- or water-resistant package. The array can be desiccated, frozen, or contacted with a storage agent (e.g., a cryoprotectant, an anti-bacterial, an anti-fungal). For example, an array can be rapidly frozen after being optionally contacted with a cryoprotectant. This step can be done at any point in the process (e.g., before or after contacting the array with an RNA polymerase; before or after contacting the array with a translation effector; or before or after washing the array). The packaged product can be supplied to a user with or without additional contents, e.g., a transcription effector, a translation effector, a vector nucleic acid, an antibody, and so forth.

In a preferred embodiment, each test amino acid sequence in the plurality of addresses is unique. For example, a test amino acid sequence can differ from all other test amino acid sequence of the plurality by 1, or more amino acid differences, (e.g., about 2, 3, 4, 5, 8, 16, 32, 64 or more differences; and, by way of example, has about 800, 256, 128, 64, or 32, 16, 8, 4, or fewer differences). In another preferred embodiment, the test amino acid sequence encoded by the nucleic acid at each address of the plurality is identical to all other test amino acid sequences in the plurality of addresses. In a preferred embodiment, the affinity tag encoded by the nucleic acid at each address of the plurality is the same, or substantially identical to all other affinity tags in the plurality of addresses. In another preferred embodiment, the nucleic acid at each address of the plurality encodes more than one affinity tag. In yet another preferred embodiment, the affinity tag encoded by the nucleic acid at an address of the plurality differs from at least one other affinity tag in the plurality of addresses.

In a preferred embodiment, the affinity tag is fused directly to the test amino acid sequence, e.g., directly amino-terminal, or directly carboxy-terminal. In another preferred embodiment, the affinity tag is separated from the test amino acid by one or more linker amino acids, e.g., 1, 2, 3, 4, 5, 6, 8, 10, 12, 20, 30 or more amino acids, preferably about 1 to 20, or about 3 to 12 amino acids. The linker amino acids can include a cleavage site, flexible amino acids (e.g., glycine, alanine, or serine, preferably glycine), and/or polar amino acids. The linker and affinity tag can be amino-terminal or carboxy-terminal to the test amino acid sequence.

The nucleic acid can be a RNA, or a DNA (e.g., a single-stranded DNA, or a double stranded DNA). In a preferred embodiment, the nucleic acid includes a plasmid DNA or a fragment thereof; an amplification product (e.g., a product generated by RCA, PCR, NASBA); or a synthetic DNA.

The nucleic acid can further include one or more of: a transcription promoter; a transcription regulatory sequence; a untranslated leader sequence; a sequence encoding a cleavage site; a recombination site; a 3′ untranslated sequence; a transcriptional terminator; and an internal ribosome entry site. In one embodiment, the nucleic acid sequence includes a plurality of cistrons (also termed “open reading frames”), e.g., the sequence is dicistronic or polycistronic. In another embodiment, the nucleic acid also includes a sequence encoding a reporter protein, e.g., a protein whose abundance can be quantitated and can provide an indication of the quantity of test polypeptide fixed to the plate. The reporter protein can be attached to the test polypeptide, e.g., covalently attached, e.g., attached as a translational fusion. The reporter protein can be an enzyme, e.g., β-galactosidase, chloramphenicol acetyl transferase, β-glucuronidase, and so forth. The reporter protein can produce or modulate light, e.g., a fluorescent protein (e.g., green fluorescent protein, variants thereof, red fluorescent protein, variants thereof, and the like), and luciferase.

The transcription promoter can be a prokaryotic promoter, a eukaryotic promoter, or a viral promoter. In a preferred embodiment, the promoter is the T7 RNA polymerase promoter. The regulatory components, e.g., the transcription promoter, can vary among nucleic acids at different addresses of the plurality. For example, different promoters can be used to vary the amount of polypeptide produced at different addresses.

In one embodiment, the nucleic acid also includes at least one site for recombination, e.g., homologous recombination or site-specific recombination, e.g., a lambda att site or variant thereof, a lox site; or a FLP site. In a preferred embodiment, the recombination site lacks stop codons in the reading frame of a nucleic acid encoding a test amino acid sequence. In another preferred embodiment, the recombination site includes a stop codon in the reading frame of a nucleic acid encoding a test amino acid sequence.

In another embodiment, the nucleic acid includes a sequence encoding a cleavage site, e.g., a protease site, e.g., a site cleaved by a site-specific protease (e.g., a thrombin site, an enterokinase site, a PreScission site, a factor Xa site, or a TEV site), or a chemical cleavage site (e.g., a methionine, preferably a unique methionine (cleavage by cyanogen bromide) or a proline (cleavage by formic acid)).

The nucleic acid can include a sequence encoding a second polypeptide tag in addition to the affinity tag. The second tag can be C-terminal to the test amino acid sequence and the affinity tag can be N-terminal to the test amino acid sequence; the second tag can be N-terminal to the test amino acid sequence, and the affinity tag can be C-terminal to the test amino acid sequence; the second tag and the affinity tag can be adjacent to one another, or separated by a linker sequence, both being N-terminal or C-terminal to the test amino acid sequence. In one embodiment, the second tag is an additional affinity tag, e.g., the same or different from the first tag. In another embodiment, the second tag is a recognition tag. For example, the recognition tag can report the presence and/or amount of test polypeptide at an address. Preferably the recognition tag has a sequence other than the sequence of the affinity tag. In still another embodiment, a plurality of polypeptide tags (e.g., less than 3, 4, 5, about 10, or about 20 tags) are encoded in addition to the first affinity tag. Each polypeptide tag of the plurality can be the same as or different from the first affinity tag.

The nucleic acid sequence can further include an identifier sequence, e.g., a non-coding nucleic acid sequence, e.g., one that is synthetically inserted, and allows for uniquely identifying the nucleic acid sequence. The identifier sequence can be sufficient in length to uniquely identify each sequence in the plurality; e.g., it is about 5 to 500, 10 to 100, 10 to 50, or about 10 to 30 nucleotides in length. The identifier can be selected so that it is not complementary or identical to another identifier or any region of each nucleic acid sequence of the plurality on the array.

The test amino acid sequence can further include a protein splicing sequence or intein. The intein can be inserted in the middle of a test amino acid sequence. The intein can be a naturally-occurring intein or a mutated intein.

The nucleic acid sequences of the plurality can be obtained from a collection of full-length expressed genes (e.g., a repository of clones), a cDNA library, or a genomic library. The test amino acid sequences can be genes expressed in a tissue, e.g., a normal or diseased tissue. The test polypeptides can be mutants or variants of a scaffold protein (e.g., an antibody, zinc-finger, polypeptide hormone etc.). In yet another embodiment, the test polypeptides are random amino acid sequences, patterned amino acids sequences, or designed amino acids sequences (e.g., sequence designed by manual, rational, or computer-aided approaches). The plurality of test amino acid sequences can include a plurality from a first source, and plurality from a second source. For example, the server can be provided with lists of test amino acid sequences associated with a diseased tissue or a first species in addition to lists of test amino acid sequences associated with a normal tissue or a second species.

The binding agent can be attached to the substrate. For example, the substrate can be derivatized and the binding agent covalent attached thereto. The binding agent can be attached via a bridging moiety, e.g., a specific binding pair. (e.g., the substrate contains a first member of a specific binding pair, and the binding agent is linked to the second member of the binding pair, the second member being attached to the substrate).

In yet another embodiment, an insoluble substrate (e.g., a bead or particle), is disposed at each address of the plurality, and the binding agent is attached to the insoluble substrate. The insoluble substrate can further contain information encoding its identity, e.g., a reference to the address on which it is disposed. The insoluble substrate can be tagged using a chemical tag, or an electronic tag (e.g., a transponder). The insoluble substrate can be disposed such that it can be removed for later analysis.

The invention also features a computer system including (i) a server storing a list of amino acid sequences and/or their descriptors, and (ii) software configured to: (1) send a list of amino acid sequence and/or their descriptors to a client; (2) receive from the client a plurality of selected amino acid sequences from the list ; and (3) interface with an array provider (e.g., a robotic system, or a technician) so as to dispose on a substrate nucleic acids encoding the selected amino acid sequences, each at a plurality of addresses.

The invention also features a method of identifying a small molecule or drug binding protein. Such proteins can include drug targets and adventitious drug-binding proteins (e.g., non-target proteins responsible for toxicity of a drug). The method includes providing or obtaining an array described herein, contacting each address of the plurality with a drug, e.g., a labeled drug. The method can further include detecting the presence of the drug at each address of the plurality. The method can also include a wash step, e.g., prior to the detecting.

The invention also features a kit that can be used to prepare a substrate described herein, e.g., a kit with one or more components for using a method described herein. In one example, the kit includes a plurality of coding nucleic acids. Each coding nucleic acid can be compatible for coupled transcription and translation. For example, the coding region is operably linked to a promoter, e.g., a T7 promoter. Each coding nucleic acid can include an anchoring agent, or the kit can include an anchoring agent that can be linked to a coding nucleic acid. The kit can also include a binding agent, e.g., that can bind to a tag encoded in at least one polypeptide encoded by one of the coding nucleic acids.

Another exemplary kit includes at least two of the following: a substrate (e.g., a planar) an anchoring agent, a transcription effector, a translation effector, and a binding agent.

In another aspect, the invention features an isolated polypeptide that comprises a fragment of Cdt1 protein. The polypeptide includes less than the entire Cdt protein, but the fragment that it does include can interact with geminin. For example, the fragment is the only part of the Cdt1protein in the isolated polypeptide. The fragment can be a 77 amino acid fragment (e.g., 135aa-212aa) or smaller. For example, the fragment includes at least a core 14 aa sequence (198-212aa) of Cdt1. The fragment can be less than 70, 60, 50, 40, 30, 20, 18, 17, 16, or 15 amino acid. In another aspect, the invention a protein, other than geminin that interacts with 198-212aa of Cdt1. For example, the protein is an antibody (or fragment thereof) or an artificial ligand (or fragment thereof). Such proteins can be isolated, e.g., by phage display, immunization, and so forth. The invention also features a method of evaluating an agent. The method includes contacting the agent (e.g., a protein or non-protein compound, e.g., candidate drug) to the isolated polypeptide that comprises a fragment of Cdt1 protein, and evaluating interaction with the isolated polypeptide. For example, the protein is a protein other than geminin or a fragment thereof. In one embodiment, the method includes (or further includes) evaluating whether interaction of the agent and the isolated polypeptide prevents binding of geminin

The term “stably attached”refers to an interaction that is not disrupted by washing under physiological conditions for one hour. Stably attached molecules can be covalently or non-covalently attached, either directly or indirectly.

The term “array,” as used herein, refers to an apparatus with a plurality of addresses. A “substrate” is an object that includes one or more surfaces, e.g., for receiving or retaining reagents. The substrate may also include one or more components that are deemed components of the substrate. For example, a substrate may include a surface coating for receiving reagents. A substrate can include a rigid support which may have such a surface coating or which may itself have a surface for receiving reagents.

A “nucleic acid programmable polypeptide array” or “NAPPA” refers to an array described herein. The term encompasses such an array at any stages of production, e.g., before any nucleic acid or polypeptide is present; when nucleic acid is disposed on the array, but no polypeptide is present; when a nucleic acid has been removed and a polypeptide is present; and so forth.

The term “address,” as referred to herein, is a positionally distinct portion of a substrate. Thus, a reagent at a first address can be positionally distinguished from a reagent at a second address. The address is located in and/or on the substrate. The address can be distinguished by two coordinates (e.g., x-y) in embodiments using two-dimensional arrays, or by three coordinates (e.g., x-y-z) in embodiments using three-dimensional arrays.

The term “substrate,” as used herein in the context of arrays (as opposed to a substrate of an enzyme), refers to a composition in or on which a nucleic acid or polypeptide is disposed. The substrate may be discontinuous. An illustrative case of a discontinuous substrate is a set of gel pads separated by a partition.

The terms “test amino acid sequence” or “test polypeptide,” as used herein, refers to a polypeptide of at least three amino acids that is translated on the array. The test amino acid sequence may or may not vary among the addresses of the array.

The term “translation effector” refers to a macromolecule capable of decoding a messenger RNA and forming peptide bonds between amino acids, either alone or in combination with other such molecules, or an ensemble of such molecules. The term encompasses ribosomes, and catalytic RNAs with the aforementioned property. A translation effector can optionally further include tRNAs, tRNA synthases, elongation factors, initiation factors, and termination factors. An example of a translation effector is a translation extract obtained from a cell.

As used herein, the term “transcription effector” refers to a composition capable of synthesizing RNA from an RNA or DNA template, e.g., a RNA polymerase.

The term “recognizes,” as used herein, refers to the ability of a first agent to bind to a second agent. Preferably, the dissociation constant or apparent dissociation constant of binding is about 100 μM, 10 μM, 1 μM, 100 nM, 10 nM, 1 nM, 100 pM, 10 pM, or less.

The term “affinity tag,” as used herein, refers to an amino acid, a peptide sequence, or a polypeptide sequence that includes a moiety capable of recognizing or reacting with a binding agent.

The term “binding agent,” as used herein, refers to a moiety, either a biological polymer (e.g., polypeptide, polysaccharide, or nucleic acid, or another chemical compound which is capable of recognizing or binding an affinity tag or which is capable of specifically reacting with an affinity tag, e.g., to form a covalent bond. The term “handle” is used synonymously with binding agent.

The term “recognition tag,” as used herein, refers to an amino acid, a peptide sequence, or a polypeptide sequence that can be detected, directly or indirectly, on the array.

As used herein, the terms “peptide,” “polypeptide,” and “protein” are used interchangeably. Generally, these terms refer to polymers of amino acids which are at least three amino acids in length.

A “unique reagent” refers to a reagent that differs from a reagent at each other address in a plurality of addresses. The reagent can differ from the reagents at other addresses in terms of one or both of: structure and function. A unique reagent can be a molecule, e.g., a biological macromolecule (e.g., a nucleic acid, a polypeptide, or a carbohydrate), a cell, or a small organic compound. In the case of biological polymers, a structural difference can be a difference in sequence at at least one position. In addition, a structural difference, e.g., for polymers having the same sequence, can be a difference in conformation (e.g., due to allosteric modification; meta-stable folding; alternative native folded states; prion or prion-like properties) or a modification (e.g., covalent and non-covalent modifications (e.g., a bound ligand))

Protein microarrays representing many different proteins, as described herein, provide a potent high-throughput tool which can greatly accelerate the study of protein function. The arrays described herein avoids the process of expressing proteins in living cells, purifying, stabilizing, and spotting them. Many NAPPA arrays, as described herein, also reduce the number of manipulations for each polypeptide, as the polypeptide can be synthesized in situ in or on the array substrate. The current invention obviates the need to purify polypeptides and to manipulate purified protein samples onto the array by the straightforward and much simpler process of disposing nucleic acids. The nucleic acids are then simultaneously transcribed/translated in a cell-free system and immobilized in situ, minimizing direct manipulation of the proteins and making this approach well suited to high-throughput applications. Further, the cotranslation of a first and second polypeptide can enhance complex formation in some cases.

In addition, the protein folding environment in cell free systems differs from the natural environment, allowing for a user to control a variety of parameters such as post-translational modifications.

The array can be easily reprogrammed to contain different sets of proteins and polypeptides.

Polypeptide arrays provide comprehensive genome-wide screens for biomolecular interactions. The arrays, as described herein, allow for the sampling of an entire library. Detecting each address of a plurality provides the certainty that each library member has been screened. Thus, complete coverage of known sequences is possible. For example, a single array containing 10,000 arrayed elements, for example, can be sufficient to yield 10,000 results (e.g., quantitative results), each result comparable with the results of other elements of the array, and potentially with a result from other arrays. High-density arrays further expand possible coverage.

Many embodiments described herein include capture of nucleic acid to a surface. Capture can be effected by a variety of means, including chemical conjugation, specific, and non-specific binding. For example, it is possible to use nucleic acid binding proteins (e.g., transcription factors, DNA binding proteins, RNA binding proteins, single strand binding proteins, promoters, inactive or mutant nucleases)

In some cases, it is useful to form protein aggregates in solution prior to binding to surface. Increase in protein concentration in spotting solution increases protein-protein interaction among the reagents. In our case, streptavidin and the antibody could interact non-specifically to form aggregates, these aggregates may increase the binding of the reagents and translated proteins to the surface. This aggregation can be achieved by using a carrier protein such as a serum albumin (e.g., HSA or BSA) which may cause a similar effect. Another alternative is to use a protein reactive crosslinker, which chemically crosslinks proteins to enhance the formation of protein aggregates. Aggregation can also be enhanced using other reagents such as dendrimers (e.g., nucleic acid or other dendrimers).

Expressed protein can be captured, e.g., by adsorption to surface, chemical linkage to surface, or by way of fusion tag (capture of fusion tag by anti-tag antibody, small molecule binding to fusion tag, polypeptide binding to fusion tag)

In some implementations, the protein array is adapted to a metal surfaces such as gold. Gold can be deposited onto a solid surface such as a plain glass slide. The surface can be treated with titanium or chromium to cause better adhesion of the gold to the surface. The surface can be treated with a number of alkyl thiol linkers terminating with different chemical moieties. Such modifications include, for example:

Exemplary scenario 1: a self assembled monolayer that is created using alkyl thiol terminating with a polyethylene glycol (PEG) (this monolayer can prevent the surface from binding to proteins).

Exemplary scenario 2: The PEG-lyated alkyl thiol can be modified to terminate with a protein binding chemical group (amines, aldehydes, epoxy, activated esters etc) which offers some degree of resistance to protein binding due to the underlying PEG groups but still binds proteins due to the reactive termini. This reduces protein adsorption but promotes protein binding via chemical linkage. For increased binding (adsorption+chemical linkage) gold slides can be treated with alkyl thiol (without PEG) terminating with either of the reactive groups (amines, aldehydes, epoxy, activated esters etc).

Exemplary scenario 3: Alkyl thiol groups from scenario 1 and 2 can be mixed in desired ratios to obtain good specific binding of proteins with low background due to non specific binding.

Exemplary scenario 4: It is also ideal to create a surface where there are reactive islands that bind only spotted sample in an inert background. This reactive island can be created in scenarios 1, 2 or 3 by forming protein aggregates (as described above). This is also true with scenario 1 which prevents protein binding to the surface except when aggregates are formed in the array sample.

Surface chemistries can be altered to create micro-3D surfaces that increase surface area for binding of proteins and other reagents. For example, the surface can be modified by chemical etching to create reactive troughs or by adding chemical moieties such as dendrimers to increase the binding capacity.

Some embodiments described herein also provide arrays and methods for detecting subtle and sensitive results. As a polypeptide species, e.g., a homogenous species, can be provided at an address without competing species, a result for the individual species can be detected. In other embodiments, arrays and methods can also including competing species for the very purpose of removing subtle results and increasing the signal of strong positives.

In sum, the arrays and methods described herein provide a versatile new platform for proteomics.

All patents, patent applications, and references cited herein are incorporated in their entireties by reference. In addition to those mentioned elsewhere in this application, the following patent applications are hereby incorporated by reference: 60/562,293, US2002-0192673-A1, PCT/US03/17979, and a PCT filed on 14 Apr. 2005 with the US Receiving Office with attorney docket number 00246-274WO1 and titled, “Nucleic-Acid Programmable Protein Arrays.” Also incorporated is Ramachandran, N. et al. 2004. Science 305:86-90.

### DESCRIPTION OF THE DRAWINGS

FIG. 1(A, B, C, D) depicts an exemplary method for providing a NAPPA array. The method includes immobilizing DNA and a binding agent (e.g., a capture antibody).

FIG. 2 depicts maps of exemplary plasmids, in which FIG. 2A shows pANT7cGST and FIG. 2B shows pANT7nHA.

FIG. 3 depicts an exemplary method for evaluating samples with a tumor cell lysate.

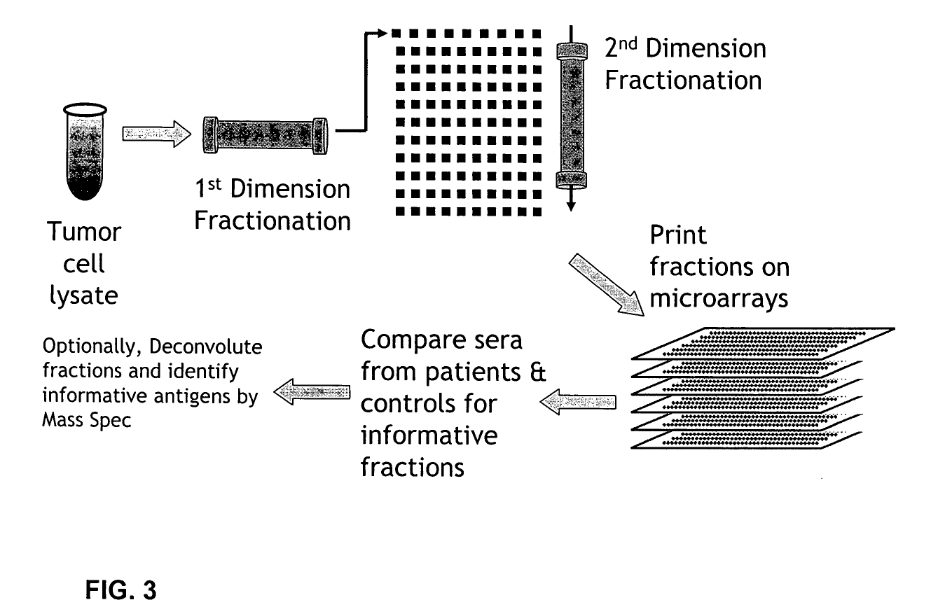

FIG. 4 depicts an exemplary method for evaluating sera for antibodies to antigens.

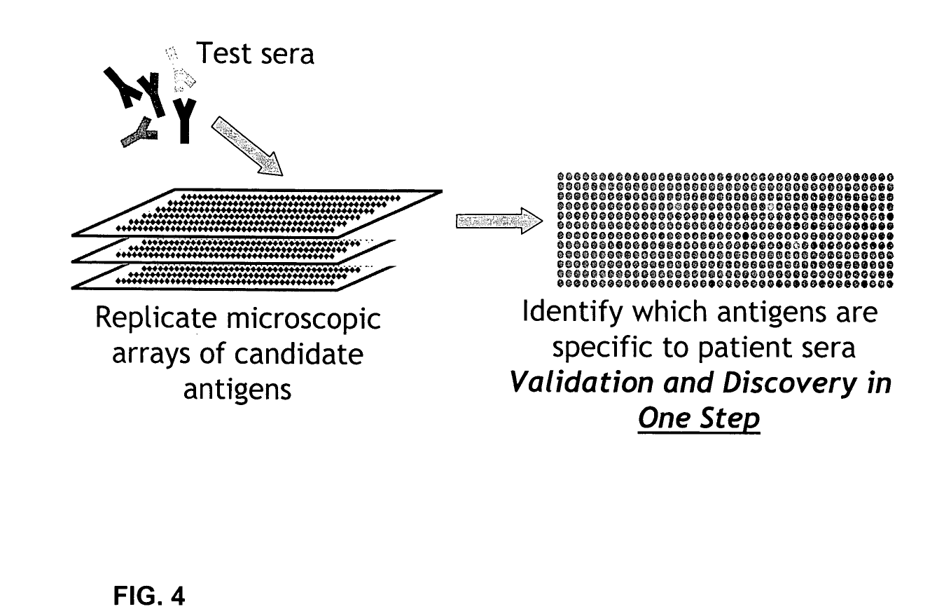

FIG. 5 depicts a surface plasmon enhanced illumination system. Light propagation depends on dielectric properties of the metal surface. The dielectric property itself depends on the mass of substance bound it. The system can be very sensitive, including single molecule detection, permits multiplexing and right resolution, and can use a small sample volume.

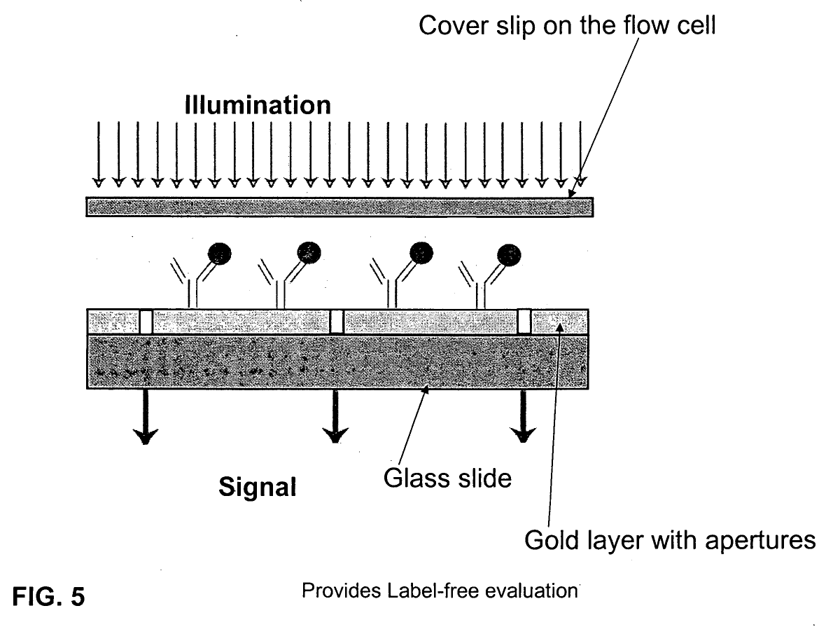

FIG. 6 depicts exemplary psoralen-linker (e.g., PEO)-biotin compounds.

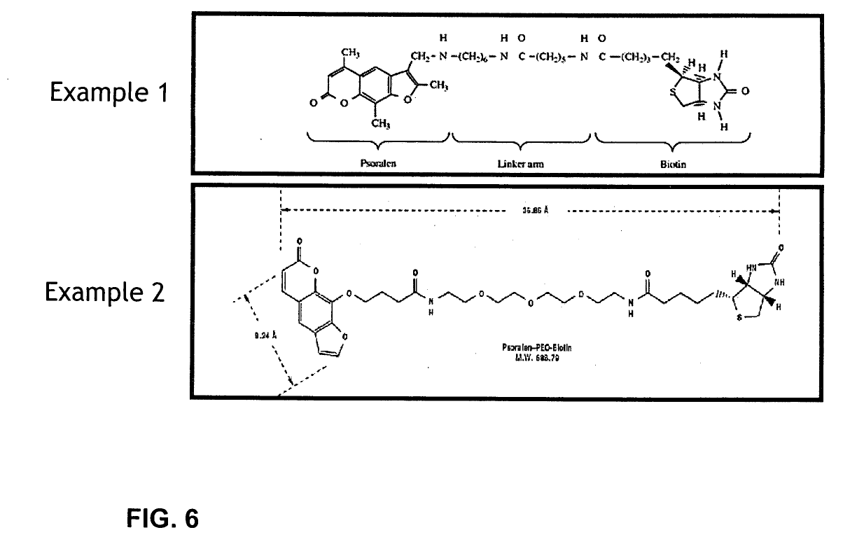

FIG. 7 depicts a miniprep method for preparing a substrate with multiple samples.

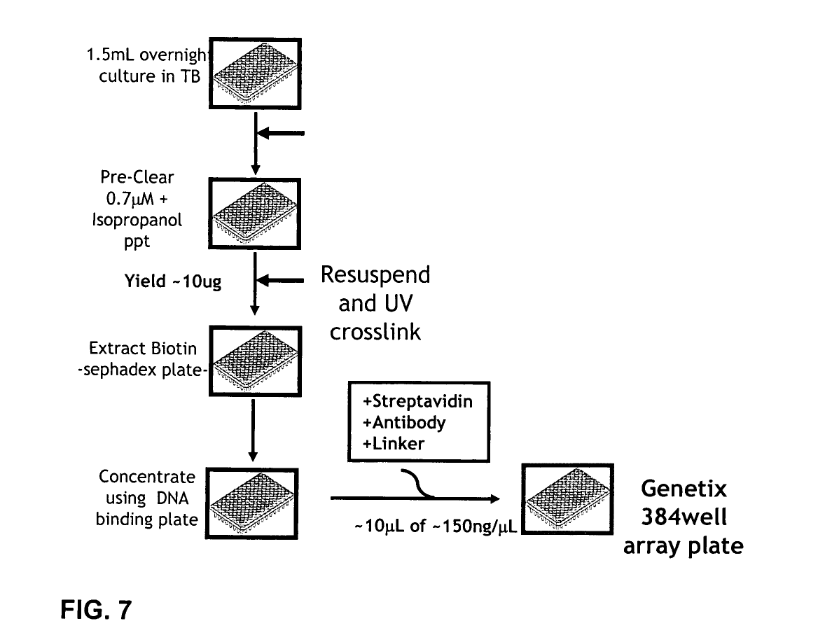

FIG. 8 depicts an exemplary substrate surface with a PEGylated alkyl chain.

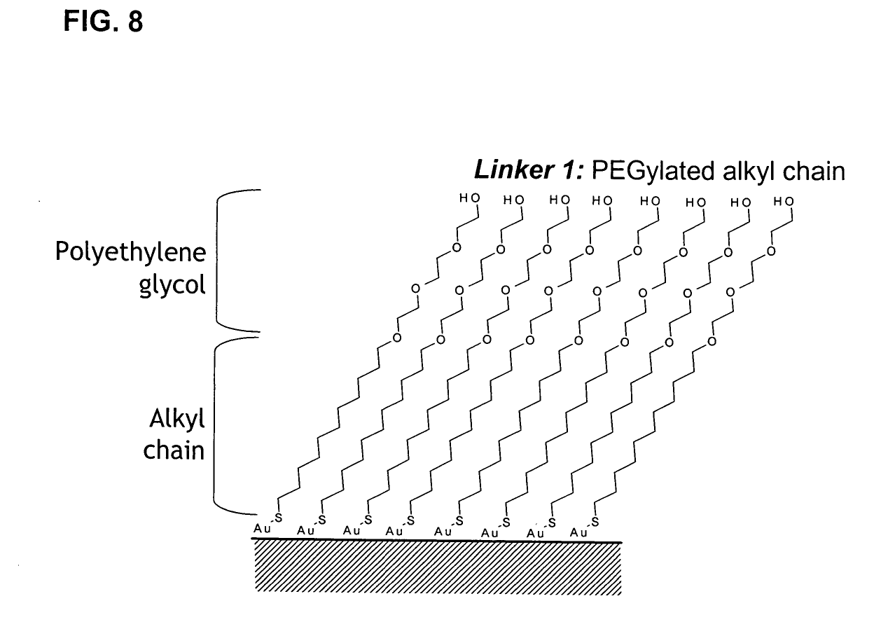

FIG. 9 depicts an exemplary substrate surface with three different exemplary linkers.

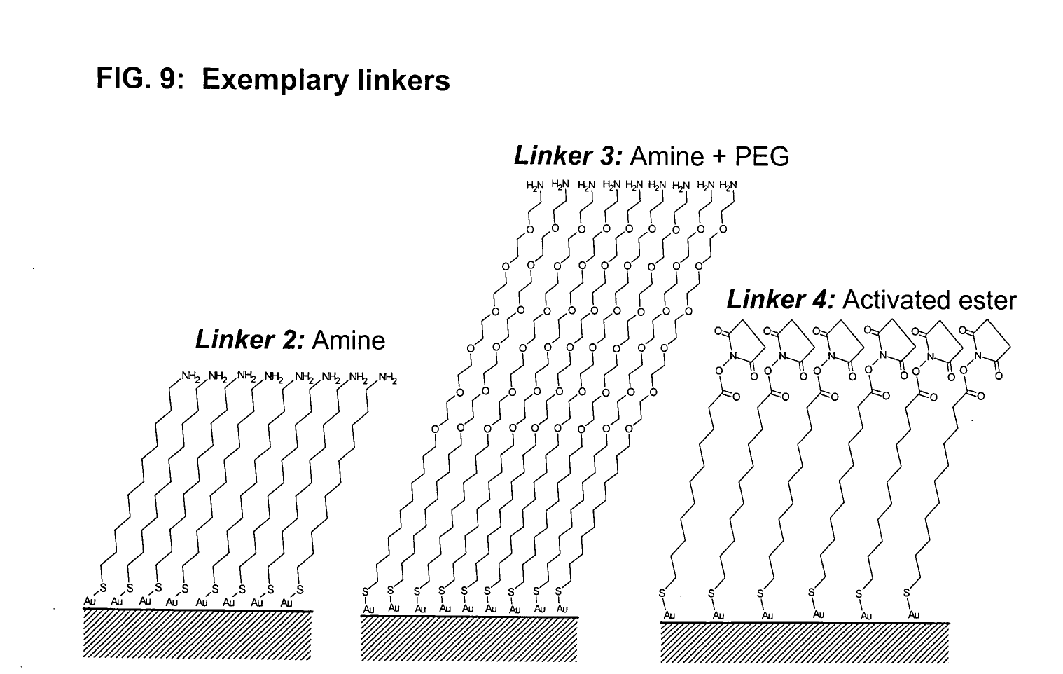

FIG. 10 depicts a substrate surface with a selective region of reactivity.

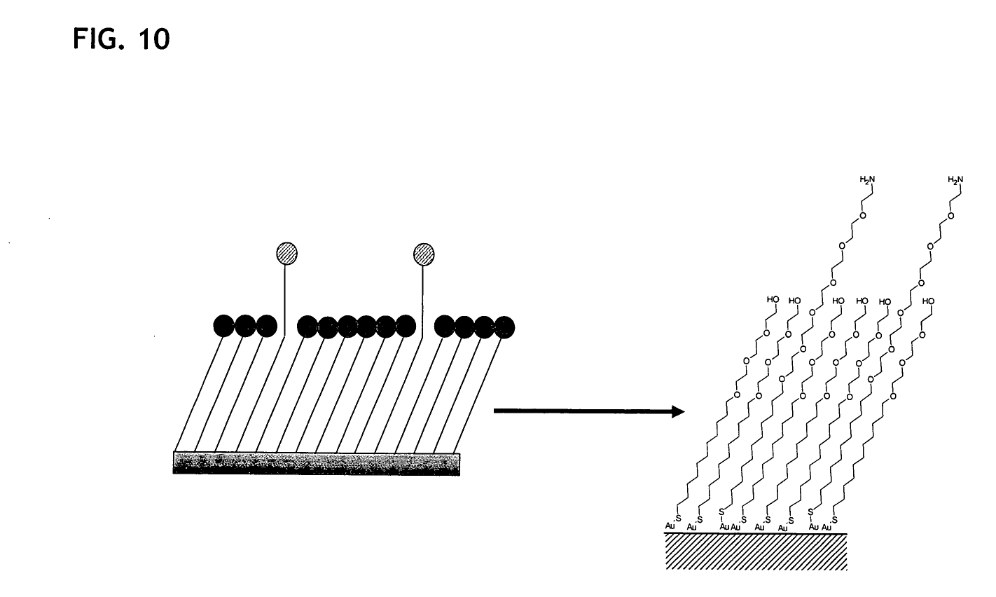

FIG. 11 depicts a substrate surface with different exemplary linkers and their contact angles.

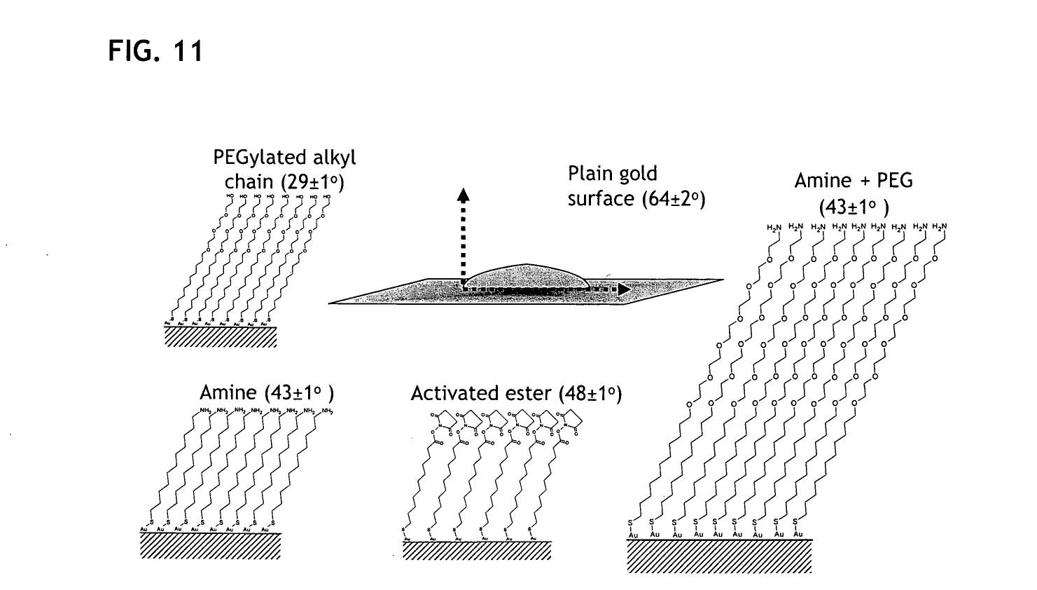

### DETAILED DESCRIPTION

The following example is a protein array that is constructed by immobilizing nucleic acids (e.g., cDNAs) encoding target proteins onto a substrate. A translation effector can be contacted to the substrate so that they are expressed and then immobilized in situ or otherwise stably attached. The proteins are typically expressed with a tag, such as a terminal tag. The tag can be used to capture the protein or to detect it. In one embodiment, the nucleic acids are stably attached to the substrate, e.g., prior to contacting the translation effector. In one embodiment, the nucleic acids are disposed on the substrate in conjunction with a binding agent that recognizes the tag.

The methods described herein can be adapted to variety of formats. For example, it can used to provide an arrayed collection of ligands, e.g., specific antibodies that can measure the presence and abundance of specific proteins (or other molecules). It can be used to provide an arrayed collection of any protein of interest, or sets of proteins, for example, to study protein function (e.g., an activity such as binding or catalytic activity), drug interactions, and protein-protein interactions. For example, arrays can be used to examine target protein interactions with other molecules, such as drugs, antibodies, nucleic acids, lipids, or other proteins. In addition, the array can be interrogated to find substrates and cofactors for enzymes.

A variety of schemes for printing the cDNAs are available. Exemplary methods include binding of different forms of naked DNA (supercoiled, nicked circular, linear) either by direct adsorption or by UV crosslinking to variously treated surfaces, the binding of DNA modified by the incorporation of surface reactive nucleotides, and the use of surface linking agents such as DNA binding proteins and/or hetero-bifunctional intercalating agents. Various exemplary approaches to immobilize nucleic acids include:

Chemically modified Nucleic Acids. Nucleic acids can be modified with reactable chemistry that covalently modifies DNA Negative nucleic acid backbone can be immobilized on to positive surface (ie aminosilane glass slide). Cleavable and non-cleavable homo-bifunctional or hetero-bifunctional linkers can be used. DNA binding functional groups can include, e.g., intercalating agents/small molecules (e.g., ethidium bromide/psoralen or nucleic acid binding molecules (chemical entities (phosphates), specific bases, major groove or minor groove binding molecules, nucleic acid binding proteins). Exemplary surface binding functional groups include sulphides/disulphides/activated esters or maleimides/biotin+avidin/ streptag+avidin/ biotin+streptavidin. Modified bases can be used. It is possible to incorporate modified bases using nick translation

Nucleic acid binding proteins can be used to immobilize nucleic acid. For example, it is possible to use proteins that bind to nucleic acid (e.g., DNA or RNA) in a sequence dependent or independent manner (e.g., histones, a transcription factor or DNA binding domain thereof Gal4 (transcription factors)), an RNA binding protein or RNA binding domain thereof. In one embodiment, the proteins are designed DNA or RNA binding proteins, e.g., zinc finger proteins. In one embodiment, adaptable vectors are used, e.g., vectors annealed to modified oligonucleotides (oligonucleotides synthesized with biotin, modified phosphates, bases, small molecules). In one embodiment, adaptable PCR products are generated using above mentioned modified oligonucleotides. Rolling circle amplification can be used to generate concatamers either on the array or prior to arraying.

Exemplary methods can include, for example, subcloning or recombinational cloning systems, or PCR generated products; various expression systems (rabbit reticulocyte, bacterial extract, wheat germ etc); proteins can be expressed with various tags for binding (GST/6xHIs/CBP/MBP etc); surface chemistry (aminosilane, aldehyde, epoxy, thiols, etc.) on glass, gold or silver coated glass, nitrocellulose, PVDF, plastics (polystyrene etc); intermediate chemistries such as BSA or dendrimers can be used as well.

The exemplary arrays described herein have a variety of applications. In one embodiment, an array can be used to build multi-component complexes. Using this approach, we were able to express multiple proteins as query and build complexes on the array itself. For example, MCM2 and Cdc6 were expressed together to evaluate ability of these components to facilitate interaction with Cdt1. Complexes can include, for example, two, three, four, or more proteins.

In another embodiment, an array can also be used in biomarker discovery. For example, patients infected with pathogens such as Pseudomonas generate antibodies to pseudomonas proteins. An array that includes all (or some fraction of, e.g., a substantial fraction) Pseudomonas proteins (e.g., produced by translating nucleic acids encoding such proteins, or proteins from any other pathogen) can be used to evaluate patient sera.

The sera of infected patients may contain antibodies to one or more of these antigens. The array would detect such antibodies and accordingly can be used as a diagnostic. The method can be used, e.g., to detect, monitor, or evaluate a subject, e.g., a subject that has a disease or disorder which can be characterized by a particular antibody, e.g., an infectious disorder, an autoimmune disorder, or a neoplastic disorder. For example, cancer patients are known to have antibodies to specific tumor antigens. By expressing a large number of genes relevant to cancer or to particular types of cancer, one identifies which tumor antigens are present. One then distinguishes between different types of cancer or different stages of cancer by analyzing the presence or absence of specific antigens or analyze patterns of detected antigens. Fragments of antigens can also be generated to map epitopes, or to provide further information.

Substrates

Materials. Both solid and porous substrates are suitable for recipients for the encoding nucleic acids described herein. A substrate material can be selected and/or optimized to be compatible with the spot size (e.g., density) required and the application.

In one embodiment, the substrate is a solid substrate. Potentially useful solid substrates include: mass spectroscopy plates (e.g., for MALDI), glass (e.g., functionalized glass, a glass slide, porous silicate glass, a single crystal silicon, quartz, UV-transparent quartz glass), plastics and polymers (e.g., polystyrene, polypropylene, polyvinylidene difluoride, poly-tetrafluoroethylene, polycarbonate, PDMS, acrylic), metal coated substrates (e.g., gold), silicon substrates, latex, membranes (e.g., nitrocellulose, nylon), a glass slide suitable for surface plasmon resonance (SPR).

In another embodiment, the substrate is porous, e.g., a gel or matrix. Potentially useful porous substrates include: agarose gels, acrylamide gels, sintered glass, dextran, meshed polymers (e.g., macroporous crosslinked dextran, sephacryl, and sepharose), and so forth.

Substrate Properties. The substrate can be opaque, translucent, or transparent. The addresses can be distributed, on the substrate in one dimension, e.g., a linear array; in two dimensions, e.g., a planar array; or in three dimensions, e.g., a three dimensional array. The solid substrate may be of any convenient shape or form, e.g., square, rectangular, ovoid, or circular. In another embodiment, the solid substrate can be disc shaped and attached to a means of rotation.

In one embodiment, the substrate contains at least 1, 10, 100, 10³, 10⁴, 10⁵, 10⁶, 10⁷, 10⁸, or 10⁹ or more addresses per cm². The center to center distance can be 5 mm, 1 mm, 100 μm, 10 μm, 1 μm, 100 nm or less. The longest diameter of each address can be 5 mm, 1 mm, 100 μm, 10 μm, 1 μm, 100 nm or less. In one embodiment, each addresses contains 0 μg, 1 μg, 100 ng, 10 ng, 1 ng, 100 pg, 10 pg, 1 pg, 0.1 pg, or less of the nucleic acid. In another embodiment, each address contains 100, 10³, 10⁴, 10⁵, 10⁶, 10⁷, 10⁸, or 10⁹ or more molecules of the nucleic acid.

The substrate can include a coated surface, e.g., a metal coated surface such as a gold surface, titanium, or chromium surface. The surface can have a contact angle of between 20-70° or between 33-50° or 50-70°, e.g., about 64°. The surface may include a polymer coat (e.g., on glass or on the metal coat). The polymer can include, e.g., a reactive end, e.g., for attachment to a protein or to an anchoring agent. Exemplary termini for polymers include amines and activated esters. Exemplary polymers include alkyl chains and polyethylene glycol, and polymers that include a region, e.g., a hydrophobic and hydrophilic region, e.g., an alkyl region and a polyethylene glycol region. The substrate can include discrete regions of reactivity, e.g., a set of selective regions that include polymers with a reactive end. The regions of reactivity can be, for example, regularly spaced from one another.

Substrate Modification. The substrate can be modified to facilitate the stable attachment of linkers, capture probes, or binding agents. Generally, a skilled artisan can use routine methods to modify a substrate in accordance with the desired application. The following are non-limiting examples of substrate modifications.

A surface can be amidated, e.g., by silylating the substrate, e.g., with trialkoxyaminosilane. Silane-treated surface can also be derivatized with homobifunctional and heterobifunctional linkers. The substrate can be derivatized, e.g., so it has a hydroxy, an amino (e.g., alkylamine), carboxyl group, N-hydroxy-succinimidyl ester, photoactivatable group, sulfhydryl, ketone, or other functional group available for reaction. The substrates can be derivatized with a mask in order to only derivatized limited areas; a chemical etch or UV light can be used to remove derivatization from selected regions.

Thus, for the preparation of glass slides, options are to derivatize the individual spots, or to derivatize the entire slide then use a physical mask, chemical etch, or UV light to cover or remove the derivatization in the areas between spots.

Partitioned Substrates. In one preferred embodiment, each address is partitioned from all other addresses in order to prevent unique molecules from diffusing to other addresses. The following are possible marcomolecules which must remain localized at the address: a template nucleic acid encoding the test amino acid sequence; amplified nucleic acid encoding the test amino acid sequence; mRNA encoding the test amino acid sequence; ribosomes, e.g., monosomes and polysomes, translating the mRNA; and the translated polypeptide.

The substrate can be partitioned, e.g., depressions, grooves, photoresist. For example, the substrate can be a microchip with microchannels and reservoirs etched therein, e.g., by photolithography. Other non-limiting examples of substrates include multi-welled plates, e.g., 96-, 384-, 1536-, 6144-well plates, and PDMS plates. Such high-density plates are commercially available, often with specific surface treatments. Depending on the optimal volume required for each application, an appropriate density plate is selected. In another embodiment, the partitions are generated by a hydrophobic substance, e.g., a Teflon mask, grease, or a marking pen (e.g., Snowman, Japan).

In one embodiment, the substrate is designed with reservoirs isolated by protected regions, e.g., a layer of photoresist. For example, for each address, a translation effector can be isolated in one reservoir, and the nucleic acid encoding a test amino acids sequence can be isolated in another reservoir. A mask can be focused or placed on the substrate, and a photoresist barrier separating the two reservoirs can be removed by illumination. The translation effector and the nucleic acid reservoirs are mixed. The method can also include moving the substrate in order to facilitate mixing. After sufficient incubation for translation to occur, and for the nascent polypeptides to bind to a binding agent, e.g., an agent attached to the substrate, additional photoresist barriers can be removed with a second mask to facilitate washing a subset or all the addresses of the substrate, or applying a second compound to each address.

Planar Substrates. In another embodiment, the addresses are not physically partitioned, but diffusion is limited on the planar substrate, e.g., by increasing the viscosity of the solution, by providing a matrix with small pore size which excludes large macromolecules, and/or by tethering at least one of the aforementioned macromolecules. Preferably, the addresses are sufficiently separated that diffusion during the time required for translation does not result in excessive displacement of the translated polypeptide to an address other than its original address on the array. In yet another embodiment, modest or even substantial diffusion to neighboring addresses is permitted. Results, e.g., a signal of a label, are processed, e.g., using a computer system, in order to determine the position of the center of the signal. Thus, by compensating for radial diffusion, the unique address of the translated polypeptide can be accurately determined.

Three-dimensional Substrates. A three-dimensional substrate can be generated, e.g., by successively applying layers of a gel matrix on a substrate. Each layer contains a plurality of addresses. The porosity of the layers can vary, e.g., so that alternating layers have reduced porosity.

In another embodiment, a three-dimensional substrate includes stacked two-dimensional substrates, e.g., in a tower format. Each two-dimensional substrate is accessible to a dispenser and detector.

Micromachined chips. Chips are made with glass and plastic materials, using rectangular or circular geometry. Wells and fluid channels are machined into the chip, and then the surfaces are derivatized. Plasmids solutions would be spotted on the chip and allowed to dry, and then a cover would be applied. Cell-free transcription/translation mix would be added via the micromachined channels. The cover prevents evaporation during incubation. A humidity-controlled chamber can be used to prevent evaporation.

CD format. A disk geometry (also termed “CD format”) is another suitable substrate for the microarray. Sample addition and reactions are performed while the disk is spinning (see PCT WO 00/40750; WO 97/21090; GB patent application 9809943.5; “The next small thing” (Dec. 9, 2000) Economist Technology Quarterly p. 8; PCT WO 91/16966; Duffy et al. (1999) Analytical Chemistry; 71, 20, (1999), 4669-4678). Thus, centrifugal force drives the flow of transcription/translation mix and wash solutions.

The disc can include sample-loading areas, reagent-loading areas, reaction chambers, and detection chambers. Such microfluidic structures are arranged radially on the disc with the originating chambers located towards the disc center. Samples from a microtiter plate can be loaded using a liquid train and a piezo dispenser. Multiple samples can be separated in the liquid train by air gaps or an inert solution. The piezo dispenser then dispenses each sample onto appropriate application areas on the CD surface, e.g., a rotating CD surface. The volume dispensed can vary, e.g., less than about 10 pL, 50 pL, 100 pL, 500 pL, 1 nL, 5 nL, or 50 nL. After entry on the CD, the centripetal force conveys the dispensed nucleic acid sample into appropriate reaction chambers. Flow between chambers can be guided by barriers, transport channels, and/or surface interactions (e.g., between the walls and the solution). The depth of channels and chambers can be adjusted to control volume and flow rate in each area.

A master CD can be made by deep reactive ion etching (DRIE) on a 6-inch silicon wafer. This master disc can be plated and used as a model to manufacture additional CDs by injection molding (e.g., Åmic AB, Uppsala, Sweden).

A stroboscopic can be used to synchronize the detector with the rotation of the CD in order to track individual detection chambers.

Transcription Effectors

RNA-directed RNA polymerases and DNA-directed RNA polymerases are both suitable transcription effectors.

DNA-directed RNA polymerases include bacteriophage T7 polymerase, phage T3, phage φII, Salmonella phage SP6, or Pseudomonas phage gh-1, as well as archeal RNA polymerases, bacterial RNA polymerase complexes, and eukaryotic RNA polymerase complexes.

T7 polymerase is a preferred polymerase. It recognizes a specific sequence, the T7 promoter (see e.g., U.S. Pat. No. 4,952,496), which can be appropriately positioned upstream of an encoding nucleic acid sequence. Although, a DNA duplex is required for recruitment and initiation of T7 polymerase, the remainder of the template can be single stranded. In embodiments utilizing other RNA polymerases, appropriate promoters and initiations sites are selected according to the specificity of the polymerase.

RNA-directed RNA polymerases can include Qβ replicase, and RNA-dependent RNA polymerase.

Translation Effectors

In one embodiment, the transcription/translation mix is in a minimal volume, and this volume is optimized for each application. The volume of translation effector at each address can be less than about 10⁻⁴, 10⁻⁵, 10⁻⁶, 10⁻⁷, 10⁻⁸, or 10⁻⁹ L. During dispensing and incubation, the array can be maintained in an environment to prevent evaporation, e.g., by covering the wells or by maintaining a humid atmosphere.

In another embodiment, the entire substrate can be coated or immersed in the translation effector. One possible translation effector is a translation extract prepared from cells. The translation extract can be prepared e.g., from a variety of cells, e.g., yeast, bacteria, mammalian cells (e.g., rabbit reticulocytes), plant cells (e.g., wheat germ), and archebacteria. In a preferred embodiment, the translation extract is a wheat germ agglutinin extract or a rabbit reticulocyte lysate. In another preferred embodiment, the translation extract also includes a transcription system, e.g., a eukaryotic, prokaryotic, or viral RNA polymerase, e.g., T7 RNA polymerase. In a preferred embodiment, the translation extract is disposed on the substrate such that it can be removed by simple washing. The translation extract can be supplemented, e.g., with additional amino acids, tRNAs, tRNA synthases, and energy regenerating systems. In one embodiment, the translation extract also include an amber, ochre, or opal suppressing tRNA. The tRNA can be modified to contain an unnatural amino acid. In another embodiment, the translation extract further includes a chaperone, e.g., an agent which unfolds or folds polypeptides, (e.g., a recombinant purified chaperones, e.g., heat shock factors, GroEL/ES and related chaperones, and so forth. In another embodiment, the translation extract includes additives (e.g., glycerol, polymers, etc.) to alter the viscosity of the extract.

Affinity Tags

An amino acid sequence that encodes a member of a specific binding pair can be used as an affinity tag. The other member of the specific binding pair is attached to the substrate, either directly or indirectly.

One class of specific binding pair is a peptide epitope and the monoclonal antibody specific for it. Any epitope to which a specific antibody is or can be made available can serve as an affinity tag. See Kolodziej and Young (1991) Methods Enz. 194:508-519 for general methods of providing an epitope tag. Exemplary epitope tags include HA (influenza haemagglutinin; Wilson et al. (1984) Cell 37:767), myc (e.g., Myc1-9E10, Evan et al. (1985) Mol. Cell. Biol. 5:3610-3616), VSV-G, FLAG, and 6-histidine (see, e.g., German Patent No. DE 19507 166).

An antibody can be coupled to a substrate of an array, e.g., indirectly using Staphylococcus aureus protein A, or streptococcal protein G. The antibody can be covalently bound to a derivatized substrate, e.g., using a crosslinker, e.g., N-hydroxy-succinimidyl ester. The test polypeptides with epitopes such as Flag, HA, or myc are bound to antibody-coated plates.

Another class of specific binding pair is a small organic molecule, and a polypeptide sequence that specifically binds it. See, for example, the specific binding pairs listed in Table 1.

TABLE 1

| Protein | Ligand |
|---|---|
| glutathione-S-transferase, | glutathione |
| chitin binding protein | chitin |
| Cellulase (CBD) | cellulose |
| maltose binding protein | amylose, or maltose |
| dihydrofolate reductases | methotrexate |
| FKBP | FK506 |

These and other specific binding pairs can also be used as an anchoring agent to anchor a nucleic acid. Other specific binding pairs include biotin and a biotin binding protein, and digoxygenin and a digoxygenin-binding antibody.

Additional art-known methods of tethering proteins, e.g., the use of specific binding pairs are suitable for the affinity or chemical capture of polypeptides on the array. Appropriate substrates include commercially available streptavidin and avidin-coated plates, for example, 96-well Pierce Reacti-Bind Metal Chelate Plates or Reacti-Bind Glutathione Coated Plates (Pierce, Rockford, Ill.). Histidine- or GST-tagged test polypeptides are immobilized on either 96-well Pierce Reacti-Bind Metal Chelate Plates or Reacti-Bind Glutathione Coated Plates, respectively, and unbound proteins are optionally washed away.

In one embodiment, the polypeptide is an enzyme, e.g., an inactive enzyme, and ligand is its substrate. Optionally, the enzyme is modified so as to form a covalent bond with its substrate. In another embodiment, the polypeptide is an enzyme, and the ligand is an enzyme inhibitor.

Yet another class of specific binding pair is a metal, and a polypeptide sequence which can chelate the metal. An exemplary pair is Ni²⁺ and the hexa-histidine sequence (see U.S. Pat. Nos. 4,877,830; 5,047,513; 5,284,933; and 5,130,663.).

In still another embodiment, the affinity tag is a dimerization sequence, e.g., a homodimerization or heterodimerization sequence., preferably a heterodimerization sequence. In one illustrative example, the affinity tag is a coiled-coil sequence, e.g., the heptad repeat region of Fos. The binding agent coupled to the array is the heptad repeat region of Jun. The test polypeptide is tethered to the substrate by heterodimization of the Fos and Jun heptad repeat regions to form a coiled-coil.

In another embodiment (see also unnatural amino acids), the affinity tag is provided by an unnatural amino acid, e.g., with a side chain having functional properties different from a naturally occurring amino acid. The binding agent attached to the substrate functions as a chemical handle to either bind or react with the affinity tag.

In a related embodiment, the affinity tag is a free cysteine which can be oxidized with a thiol group attached to the substrate to create a disulfide bond that tethers the test polypeptide.

Disposal of Nucleic Acid Sequences on Arrays

The substrate and the liquid-handling equipment are selected with consideration for required liquid volume, positional accuracy, evaporation, and cross-contamination. The density of spots can depend on the liquid volume required for a particular application, and on the substrate, e.g., how much a liquid drop spreads on the substrate due to surface tension, and the positional accuracy of the dispensing equipment.

Numerous methods are available for dispensing small volumes of liquid onto substrates. For example, U.S. Pat. No. 6,112,605 describes a device for dispensing small volumes of liquid. U.S. Pat. No. 6,110,426 describes a capillary action-based method of dispensing known volumes of a sample onto an array. The dispense material can include a mixture described herein, e.g., a nucleic acid and a binding agent, or a nucleic acid physically associated with an attachment moiety and, optionally, a binding agent.

Nucleic acid spotted onto slides can be allowed to dry by evaporation. Dry air can be used to accelerate the process.

Capture Probes. The substrate can include an attached nucleic acid capture probe at each address. In one aspect, capture probes can be used create a self-assembling array. A unique capture probe at each address selectively hybridizes to a nucleic acid encoding a test amino acid sequence, thereby organizing each encoding nucleic acid to a unique address. The capture nucleic acid can be covalently attached or bound, e.g., to a polycationic surface on the substrate.

The capture probe can itself be synthesized in situ, e.g., by a light-directed method (see, e.g., U.S. Pat. No. 5,445,934), or by being spotted or disposed at the addresses. The capture probe can hybridize to the nucleic acid encoding the test polypeptide. In a preferred embodiment, the capture probe anneals to the T7 promoter region of a single stranded nucleic acid encoding the test amino acid sequence. In another embodiment, the capture probe is ligated to the encoding nucleic acid sequence. In yet another embodiment, the capture probe is a padlock probe. In still another embodiment, the capture probe hybridizes to a nucleic acid encoding a test amino acid sequence, e.g., a unique region of the nucleic acid, or to a nucleic acid sequence tag provided on the nucleic acid for the purposes of identification.

Disposed Insoluble Substrates

One or more insoluble substrates having a binding agent attached can be disposed at each address of the array. The insoluble substrates can further include a unique identifier, such as a chemical, nucleic acid, or electronic tag. Chemical tags, e.g., such as those used for recursive identification in “split and pool” combinatorial syntheses. Kerr et al. (1993) J. Am. Chem. Soc., 115:2529-2531) Nikolaiev et al. ((1993) Peptide Res. 6, 161-170) and Ohlmeyer et al.((1993) Proc. Natl. Acad. Sci. USA 90:10922-10926) describe methods for coding and decoding such tags. A nucleic acid tag can be a short oligonucleotide sequence that is unique for a given address. The nucleic acid tag can be coupled to the particle. In another embodiment, the encoding nucleic acid provides a unique identifier. The encoding nucleic acid can be coupled or attached to the particle. Electronic tags include transponders as mentioned below. The insoluble substrate can be a particle (e.g., a nanoparticle, or a transponder), or a bead.

Beads. The disposed particle can be a bead, e.g., constructed from latex, polystyrene, agarose, a dextran (sepharose, sephacryl), and so forth.

Transponders. U.S. Pat. No. 5,736,332 describes methods of using small particles containing a transponder on which a handle or binding agent can be affixed. The identity of the particle is discerned by a read-write scanner device which can encode and decode data, e.g., an electronic identifier, on the particle (see also Nicolaou et al. (1995) Angew. Chem. Int. Ed. Engl. 34:2289-2291). Test polypeptides are bound to the transponder by attaching to the handle or binding agent.

Disposed Nucleic Acid Sequences

Any appropriate nucleic acid for translation can be disposed at an address of the array. The nucleic acid can be an RNA, single stranded DNA, a double stranded DNA, or combinations thereof. For example, a single-stranded DNA can include a hairpin loop at its 5′ end which anneals to the T7 promoter sequence to form a duplex in that region. The nucleic acid can be an amplification products, e.g., from PCR (U.S. Pat. Nos. 4,683,196 and 4,683,202); rolling circle amplification (“RCA,” U.S. Pat. No. 5,714,320), isothermal RNA amplification or NASBA (U.S. Pat. Nos. 5,130,238; 5,409,818; and 5,554,517), and strand displacement amplification (U.S. Pat. No. 5,455,166).

In one embodiment, the sequence of the encoding nucleic acid is known prior to being disposed at an address. In another embodiment, the sequence of the encoding nucleic acid is unknown prior to disposal at an address. For example, the nucleic acid can be randomly obtained from a library. The nucleic acid can be sequenced after the address on which it is placed has been identified as encoding a polypeptide of interest.

Amplification in Situ

A nucleic acid disposed on the array can be amplified directly on the array, by a variety of methods, e.g., PCR (U.S. Pat. Nos. 4,683,196 and 4,683,202); rolling circle amplification (“RCA,” U.S. Pat. No. 5,714,320), isothermal RNA amplification or NASBA, and strand displacement amplification (U.S. Pat. No. 5,455,166).

Isothermal RNA amplification or “NASBA” is well described in the art (see, e.g., U.S. Pat. Nos. 5,130,238; 5,409,818; and 5,554,517; Romano et al. (1997) Immunol Invest. 26:15-28; in technical literature for “RnampliFire™” Qiagen, Calif.). Isothermal RNA amplification is particularly suitable as reactions are homogenous, can be performed at ambient temperatures, and produce RNA templates suitable for translation.

Vectors for Expression

Coding regions of interest can be taken from a source plasmid, e.g., containing a full length gene and convenient restriction sites, or sites for homologous or site-specific recombination, and transferred to an expression vector. The expression vector includes a promoter and an operably linked coding region, e.g., encoding an affinity tag, such as one described herein. The tag can be N or C terminal. The vector can carry a cap-independent translation enhancer (CITE, or IRES, internal ribosome entry site) for increased in vitro translation of RNA prepared from cloned DNA sequences. The fusion proteins will be generated with commercially available in vitro transcription/translation kits such as the Promega TNT Coupled Reticulocyte Lysate Systems or TNT Coupled Wheat Germ Extract Systems. Cell-free extracts containing translation component derived from microorganisms, such as a yeast, or a bacteria, can also be used.

In addition, the vector can include a number of regulatory sequences such as a transcription promoter; a transcription regulatory sequence; a untranslated leader sequence; a sequence encoding a protease site; a recombination site; a 3′ untranslated sequence; a transcriptional terminator; and an internal ribosome entry site.

The vector or encoding nucleic acid can also include a sequence encoding an intein. Methods of using inteins for the regulated removal of an intervening sequence are described, e.g., in U.S. Pat. Nos. 5,496,714 and 5,834,247. Inteins can be used to cyclize, ligate, and/or polymerize polypeptides, e.g., as described in Evans et al. (1999) J Biol Chem 274:3923 and Evans et al. (1999) J Biol Chem 274:18359.

Exemplary Useful Sequences

Naturally occurring sequences. Useful encoding nucleic acid sequence for creating arrays include naturally occurring sequences. Such nucleic acids can be stored in a repository, see below. Nucleic acid sequences can be procured from cells of species from the kingdoms of animals, bacteria, archebacteria, plants, and fungi. Non-limiting examples of eukaryotic species include: mammals such as human, mouse (Mus musculus), and rat; insects such as Drosophila melanogaster; nematodes such as Caernorhabditis elegans; other vertebrates such as Brachydanio rerio; parasites such as Plasmodium falciparum, Leishmania major; fungi such as yeasts, Histoplasma, Cryptococcus, Saccharomyces cerevisiae, Schizosaccharomyces pombe, Pichia pastoris and the like); and plants such as Arabidoposis thaliana, rice, maize, wheat, tobacco, tomato, potato, and flax. Non-limiting examples of bacterial species include E. coli, B. subtilis, Mycobacterium tuberculosis, Pseudomonas aeriginosa, Vibrio cholerae, Thermatoga maritime, Mycoplasma pneumoniae, Mycoplasma genitalium, Helicobacter pylori, Neisseria meningitidis, and Borrelia burgdorferi. In additional, amino acid sequence encoded by viral genomes can be used, e.g., a sequence from rotavirus, hepatitis A virus, hepatitis B virus, hepatitis C virus, herpes virus, papilloma virus, or a retrovirus (e.g., HIV-1, HIV-2, HTLV, SIV, and STLV).

In a preferred embodiment, a cDNA library is prepared from a desired tissue of a desired species in a vector described herein. Colonies from the library are picked, e.g., using a robotic colony picker. DNA is prepared from each colony and used to program an array.

Artificial sequences. The encoding nucleic acid sequence can encode artificial amino acid sequences. Artificial sequences can be randomized amino acid sequences, patterned amino acid sequence, computer-designed amino acid sequences, and combinations of the above with each other or with naturally occurring sequences. Cho et al. (2000) J Mol Biol 297:309-19 describes methods for preparing libraries of randomized and patterned amino acid sequences. Similar techniques using randomized oligonucleotides can be used to construct libraries of random sequences. Individual sequences in the library (or pools thereof) can be used to program an array.

Dahiyat and Mayo (1997) Science 278:82-7 describe an artificial sequence designed by a computer system using the dead-end elimination theorem. Similar systems can be used to design amino acid sequences, e.g., based on a desired structure, such that they fold stably. In addition, computer systems can be used to modify naturally occurring sequences in order

Mutagenesis. The array can be used to display the products of a mutagenesis or selection. Examples of mutagenesis procedures include cassette mutagenesis (see e.g., Reidhaar-Olson and Sauer (1988) Science 241:53-7), PCR mutagenesis (e.g., using manganese to decrease polymerase fidelity), in vivo mutagenesis (e.g., by transfer of the nucleic acid in a repair deficient host cell), and DNA shuffling (see U.S. Pat. Nos. 5,605,793; 5,830,721; and 6,132,970). Examples of selection procedures include complementation screens, and phage display screens

In addition, more methodical variation can be achieved. For example, an amino acid position or positions of a naturally occurring protein can be systematically varied, such that each possible substitution is present at a unique position on the array. For example, the all the residues of a binding interface can be varied to all possible other combinations. Alternatively, the range of variation can be restricted to reasonable or limited amino acid sets.

Collections. Additional collections include arrays having at different addresses one of the following combinations: combinatorial variants of a bioactive peptide; specific variants of a single polypeptide species (splice variants, isolated domains, domain deletions, point mutants); polypeptide orthologs from different species; polypeptide components of a cellular pathway (e.g., a signalling pathway, a regulatory pathway, or a metabolic pathway); and the entire polypeptide complement of an organism.

Some exemplary proteins that can be encoded by a nucleic acid disposed on the array include, e.g., ALCAM, BCAM, CADs, EpCAM, ICAMs, Cadherins, Selectins, MCAM, NCAM, PECAM and VCAM); angiogenic factors (e.g. Angiogenin, Angiopoietins, Endothelins, Flk-1, Tie-2 and VEGFs); binding proteins (e.g. IGF binding proteins); cell surface proteins (e.g. B7s, CD14, CD21, CD28, CD34, CD38, CD4, CD6, CD8a, CD64, CTLA-4, decorin, LAMP, SLAM, ST2 and TOSO); chemokines (e.g. 6Ckine, BLC/BCA-1, ENA-78, eotaxins, fractalkine, GROs, HCCs, MCPs, MDC, MIG, MIPs, MPIF-1, PARC, RANTES, TARK, TECK and SDF-1); chemokine receptors (e.g. CCRs, CX3CR-1 and CXCRs); cytokines and their receptors (e.g. Epo, Flt-3 ligand, G-CSF, GM-CSF, interferons, IGFs, IK, leptin, LIF, M-CSF, MIF, MSP, oncostatin M, osteopontin, prolactin, SARPs, PD-ECGF, PDGF A and B chains, Tpo, TIGF and PREF-1, AXL, interferon receptors, c-kit, c-met, Epo R, Flt-s/Flk-2 R, G-CSF R, GM-CSF R, etc.); ephrin and ephrin receptors; epidermal growth factors (e.g. amphiregulin, betacellulin, cripto, erbB 1, erbB3, erbB4, HB-EGF and TGF-α); fibroblast growth factors (FGFs) and receptors (FGFRs); platelet-derived growth factors (PDGFs) and receptors (PDGFRs); transforming growth factors beta (TGFs-β, e.g. activins, bone morphogenic proteins (BMPs) and receptors (BMPRs), endometrial bleeding associated factor (EBAF), inhibin A and MIC-1); transforming growth factors alpha (TGFs-α); insulin-like growth factors (IGFs); integrins (alphas and betas); interleukins and interleukin receptors; neurotrophic factors (e.g. BDNF, b-NGF, CNTF, CNTF Ra, GDNF, GRFas, midkine, MUSK, neuritin, neuropilins, NGF R, NT-3, semaphorins, TrkA, TrkB and TrkC); interferons and their receptors; orphan receptors (e.g. Bob, ChemR23, CKRLs, GRPs, RDC-1 and STRL33/Bonzo); proteases and release factors (e.g. matrix metalloproteinases (MMPs), caspases, furin, plasminogen, SPC4, TACE, TIMPs and urokinase R); T cell receptors; MHC peptides; MHC peptide complexes; B cell receptors; intracellular adhesion molecules (ICAMs); Toll-like receptors (TLRs; recognize extracellular pathogens, such as pattern recognition receptors (PRR receptors) and PPAR ligands (peroxisome proliferative-activated receptors); ion channel receptors; neurotransmitters and their receptors (e.g. receptors for nicotinic acetylcholine, acetylcholine, serotonin, .gamma.-aminobutyrate (GABA), glutamate, aspartate, glycine, histamine, epinephrine, norepinephrine, dopamine, adenosine, ATP and nitric oxide); muscarinic receptors; small molecule receptors (e.g. NO and CO₂ receptors); peptide hormones and their receptors (e.g. human placental lactogen, prolactin, gonadotropins, corticotropins, calcitonin, insulin, glucagon, somatostatin, gastrin and vasopressin); tumor necrosis factors (TNFs, e.g., CD27, CD27L, CD30, CD30L, CD40, CD40L, DR-3, Fas, FasL, HVEM, osteoprotegerin, RANK, TRAILs, TRANCE) and their receptors; nuclear factors; and G proteins and G protein coupled receptors (GPCRs), and soluble fragments thereof. Other proteins include the anti-Her-2 monoclonal antibody trastuzumab (HERCEPTIN®) and the anti-CD20 monoclonal antibodies rituximab (RITUXAN®), tositumomab (BEXXAR™) and Ibritumomab (ZEVALIN™), the anti-CD52 monoclonal antibody Alemtuzumab (CAMPATH™), the anti-TNFα. antibodies infliximab (REMICADE™) and CDP-571 (HUMICADE®), the monoclonal antibody edrecolomab (PANOREX®), the anti-CD3 antibody muromab-CD3 (ORTHOCLONE®), the anti-IL-2R antibody daclizumab (ZENAPAX®), the omalizumab antibody against IgE (XOLAIR®), the monoclonal antibody bevacizumab (AVATIN™), small molecules such as erlotinib-HCl (TARCEVA™) and others that bind to receptors or cell surface proteins.

Repositories of Nucleic Acids

The arrays described herein can be produced from nucleic acid sequences in a large repository. For example, commercial and academic institutions are providing large-scale repositories of all known and/or available genes and predicted open reading frames (ORFs) from human and other commonly studied organism, both eukaryotic, prokaryotic, and archeal. For example, the collection can contain 500, 1,000, 10,000, 20,000, 30,000 50,000, 100,000 or more full-length sequences. One example of such a repository is the FLEX (Full Length EXpression) Repository (Harvard Institute of Proteomics, Harvard Medical School, Boston, Mass.). The repository can be maintained as a clone bank, e.g., of frozen bacteria transformed with a plasmid containing a full-length coding region. A central computing unit can control access and information regarding each full-length coding region. For example, each clone can be accessible to a robot and can be tracked and verified, e.g., by a locator (e.g., a bar code, a transponder, or other electronic identifier). Thus, a desired construct can be obtained from the repository through a network-based user interface without manual intervention. The computing unit can also collate and maintain any information gathered by experimentation or by other databases regarding each clone. For example, each sample can be linked to a network-accessible relational database that tracks its bioinformatics data, storage location and cloning history, as well as any relevant links to other biological databases.

The clones in the collection can be maintained and produced in a format compatible with a recombinational cloning system that enables automated directional and in-frame shuttling of genes into virtually any expression or functional vector, obviating the need for standard subcloning approaches. The conventional production of various expression constructs requires a slow process of subcloning using restriction enzymes and ligases. Because of the variability in available restriction sites, each gene requires an individualized cloning strategy that may need to be altered for every different expression assay depending on the available sites in the necessary plasmids. In contrast, recombinational cloning, described below, is a novel alternative technique that is highly efficient, rapid, and easily scaled for high-throughput performance.

Recombinational Cloning

Methods for recombinational cloning are well known in the art (see e.g., U.S. Pat. No. 5,888,732; Walhout et al. (2000) Science 287:116; Liu et al. (1998) Curr. Biol. 8(24):1300-9.). Recombinational cloning exploits the activity of certain enzymes that cleave DNA at specific sequences and then rejoin the ends with other matching sequences during a single concerted reaction.

U.S. Pat. No. 5,888,732 describes a system based upon the site-specific recombination of bacteriophage lambda and uses double recombination. In double recombination, any DNA fragment that resides between the two different recombination sites will be transferred to a second vector that has the corresponding complementary sites. The system relies on two vectors, a master clone vector and a target vector. The one harboring the original gene is known as the master clone. The second plasmid is the target vector, the vector required for a specific application, such as a vector described herein for programming an array. Different versions of the expression vectors are designed for different applications, e.g., with different affinity and/or recognition tags, but all can receive the gene from the master clone. Site-specific recombination sites are located within the expression vector at a location appropriate to receive the coding nucleic acid sequence harbored in the master clone. Particular attention is given to insure that the reading frame is maintained for translation fusions, e.g., to an affinity or recognition tag. To shuttle the gene into the target vector, the master clone vector containing a nucleic acid sequence of interest and the target vector are mixed with the recombinase.

The mixture is transformed into an appropriate bacterial host strain. The master clone vector and the target vector can contain different antibiotic selection markers. Moreover, the target vector can contain a gene that is toxic to bacteria that is located between the recombination sites such that excision of the toxic gene is required during recombination. Thus, the cloning products that are viable in bacteria under the appropriate selection are almost exclusively the desired construct. In practice, the efficiency of cloning the desired product approaches 100%.

To construct the repository, a computer system can be used to automatically design primers based on sequence information, e.g., in a database. Each gene is amplified from an appropriate cDNA library using PCR. The recombination sequences are incorporated into the PCR primers so the amplification product can be directly recombined into a master vector. As described above, because the master vector carries a toxic gene that is lost only after successful recombination, the desired master clone is the only viable product of the process. Once in the master vector, the gene can be verified, e.g., by sequencing methods, and then shuttled into any of the many available expression vectors.

In a preferred embodiment, each gene is cloned twice, i.e., into two master vectors. In one clone, the stop codon is removed to provide for carboxy-terminal fusions. In the other clone, the native stop codon is maintained. This is particularly important for polypeptides whose function is dependent on the integrity of their carboxy-terminus.

Genes in the repository are thus suitable prepared for analysis in activity screens and functional genomics experiments using the NAPPA array. Because of the ease of shuttling multiple genes to any expression vector en masse, these clones can be prepared in multiple array formats, such as those described herein, for a variety of functional assays.

Liu et al. (1998) Curr. Biol. 8:1300 describe a Cre-lox based site-specific recombination system for the directional cloning of PCR products. This system uses Cre-Lox recombination and a single recombination site. Here again the master clone is mixed with a target vector and recombinases. However, instead of swapping fragments, the recombination product is a double plasmid connected at the recombination site. This then juxtaposes one end of the gene (whichever end was near the recombination site) with the desired signals in the expression plasmid.

The clone can include a vector sequence and a full-length coding region of interest. The coding region can be flanked by marker sequences for site-specific recombinational cloning, e.g., Cre-Lox sites, or lambda int sites (see, e.g., Uetz et al. (2000) Nature 403:623-7). Also, the coding region can be flanked by marker sequences for homologous recombination (see, e.g., Martzen et al. (1999) Science 286:1153-5). For homologous recombination almost any sequence can be used that is present in the vector and appended to the coding region. For example, the sequence can encode an epitope or protease cleavage site. After recombination, the full-length coding region can be efficiently shuttled into a recipient plasmid of choice. For example the recipient plasmid can have nucleic acid sequences encoding any one or more of the following optional features: an affinity tag, a protease site, and an enzyme or reporter polypeptide. The recipient plasmid can also have a promoter for RNA polymerase, e.g., the T7 RNA polymerase promoter and/or regulatory sites; a transcriptional terminator; a translational enhancer e.g., a Shine-Dalgarno site, or a Kozak consensus sequence.

Pool Method

A large number of proteins can be screened in one or more passes by the following pooling method. The method uses a first array wherein each address includes a pool of encoding nucleic acid sequences. Addresses identified in a screen with the first array are optionally further analyzed by splitting the pool into different addresses in at least a second array.

Each address of the first array includes a plurality of nucleic acid sequences, each encoding a unique test amino acid sequence and an affinity tag. Thus, each address encodes a pool of test polypeptides. The pools can be random collections, e.g., fractions of cDNA library, or specific collections of sequence, e.g., each address can contain a family of related or homologous sequences, a set of sequence expressed under similar conditions, or a set of sequences from a particular species (e.g., of pathogens). Preferably, a test polypeptide is encoded at only one address of the array.

An interaction detected at a given address by the presence of the second amino acid sequence at an addresses can be further analyzed (e.g., deconvolved) by providing a second array, similar to the first, however, each address containing a nucleic acid sequence encoding a single test polypeptide, the test polypeptide being one of the plurality of test polypeptides at the given address of the first array.

However, arrays with specific collections may not require using a second array. For example, in diagnostic applications, it may suffice to merely identify a collection of sequences.

In another embodiment, an array is used to deconvolve a pool of library sequence identified in a screen that did not rely on arrays to screen initial pools. For example, Kirschner and colleagues describe an in vitro screening method to identify protein interaction partners using radioactively labeled protein pools derived from small pool cDNA libraries (Lustig et al. (1997) Methods Enzymol. 283:83-99.). Individual members of such pools can be identified using an array in which unique nucleic acid components of the pool are disposed at unique addresses on the NAPPA platform. An array of sufficient density obviates the need to iteratively subdivide the pool.

In yet another embodiment, the substrate includes a plurality of nucleic acids at each address. The plurality of nucleic acid sequence encodes a different plurality of test polypeptides from the plurality at another address. Each plurality is such that it encodes the components of a protein complex, e.g., a heterodimer, or larger multimer. Exemplary protein complexes include multi-component enzymes, cytoskeletal components, transcription complexes, and signalling complexes. The array can have a different protein complex present at each address, or variation in protein complex composition at each address (e.g., for complexes with optional components, the presence or absence of such components can be varied among the addresses). One or more members of the plurality of test polypeptides can have an affinity tag, preferably just one member has an affinity tag.

In still another embodiment, the plurality of encoding nucleic acids at each address are selected by a computer program which identifies groups of encoding nucleic acids for each address such that if an address is identified, the relevant polypeptide sequence can be determined with little or no ambiguity. For example, for MALDI-TOF detection methods, encoding nucleic acid are grouped such that masses of peptide fragments (e.g., from protease digestions) of the polypeptides encoded by the plurality are distinct, or non-overlapping. Thus, detection of a peptide mass from time-of-flight data at an address would unambiguously identify the relevant polypeptide.

Unnatural Amino Acids PCT WO90/05785 describes the use of in vitro translation extracts to include unnatural amino acids at defined positions within a polypeptide. In this method, a stop codon, e.g., an amber codon, is inserted in the nucleic acid sequence encoding the polypeptide at the desired position. An amber-suppressing tRNA with an unnatural amino acid is prepared artificially and included in the translation extract. This method allows for alteration at any given position of a polypeptide sequence to an artificial amino acids, e.g., an amino acids with chemical properties not available from the standard amino acid set.

In a preferred embodiment, the amber-suppressing tRNA has an unnatural amino acid with a keto group. Keto groups are particularly useful chemical handles as they are stable in an unprotected form in cell extracts, and able to react with hydrazide and alkoxyamines to form hydrazones and oximes (Cornish et al. (1996) JACS 118:8150). Thus, the amber codon can be used as an affinity tag to attach translated proteins to a hydrazide attached to the substrate.

Exemplary General Applications

The polypeptide arrays described herein can be used in a number of applications. Non-limiting examples are described as follows. The regulation of cellular processes, including control of gene expression, can be investigated by examining protein-protein, protein-peptide, and protein-nucleic acid interactions; antibodies can be screened against an array of potential antigens for profiling antibody specificity or to search for common epitopes; proteins can be assayed for discrete biochemical activities; and the disruption of protein-ligand interactions by synthetic molecules or the direct detection of protein-synthetic molecule interactions can aid drug discovery. Given the versatility of programming the array, elements at each address are easily customized as appropriate for the desired application.

Protein arrays can be used to characterize biomarkers and autoantibodies. For example, nucleic acids can be bound and expressed on an array surface and screened with patient serum to identify novel immunodominant antigens. A patient's immune system can produce humoral responses to antigens, these antigens may be proteins that are normally found in the body but depending on their pathophysiology there may be alterations in protein expression, mutation, degradation, or localization which may make the protein immunogenic. This can be used to evaluate subject having or suspected of having autoimmune diseases. The humoral response can also be proteins that are either pathogenic or viral in origin. Therefore by expressing potential antigens one could screen with patient sera and identify immunodominant antigens derived from tumors (breast, colorectal, prostate etc), autoimmune rheumatic diseases, pathogenic, and/or viral. The identification of immunodominant antigens with high sensitivity and specificity can be used for early detection of disease, to develop vaccines, and monitor disease progression and therapy. For some of these applications, the protein can be configured to include evaluated antigens to be used as a diagnostic tool.

Protein arrays can be used for analysis using label free systems, such as mass spectrometry, calorimetry, and/or surface plasmon resonance. Most of these applications are implemented using substates that have specific surface chemistry such as surfaces with properties with suitable conductivity and ability to generate plasmons. An exemplary protein array has been adapted to the gold surface as described above which satisfies the demands of these label free detection systems.

The arrays can be probed with complex protein mixtures such as cell lysates, tissue, patient sera, etc. In this approach, multiple binding events may take place at each feature of the array resulting in varying composition and amounts of bound material from feature to feature. Using label free systems these binding events can be measured and in some cases the identity, relative amounts and kinetics of the binding can be determined. This information can be used to generate patterns which can then be used to generate signatures that are specific to the sample. The ability to create unique signatures may help discern the presence of disease, biological agents, or changes in biological response.

On the other hand, proteins arrays can be probed with a defined query rather than a complex mixture. This avoids the need for labeling query molecules such as small molecules, peptides, nucleic acids which may affect their binding kinetics. Using this approach one can identify both specific and non specific interactions with proteins on the array. For example this could be applied to determine specificity of antibodies, small molecules, enzymes, receptors as well as any off target interactions. Moreover, fragments of the binding proteins can be expressed to identify the interacting domains.

Protein Activity Detection

A nucleic acid programmable array can be used to detect a specific protein activity. Each address of the array is contacted with the reagents necessary for an activity assay. Then an address having the activity is detected to thereby identify a protein having a desired activity. An activity can be detected by assaying for a product produced by a protein activity or by assaying for a substrate consumed by a protein activity.

Protein Interaction Detection

A nucleic acid programmable array can be used to detect protein-protein interactions. Moreover, the array can be used to generate a complete matrix of protein-protein interactions such as for a protein-interaction map (see, e.g., Walhout et al., Science 287: 116-122, 2000; Uetz et al., Nature 403, 623-631, 2000); and Schwikowski (2000) Nature Biotech. 18:1257). The matrix can be generate for the complete complement of a genome, proteins known or suspected to be co-regulated, proteins known or suspected to be in a regulatory network, and so forth.

The detection of protein-protein interactions, e.g., between a first and a second protein, entails providing at an address a nucleic acid encoding the first polypeptide and an affinity tag, and a nucleic acid encoding a second polypeptide and a recognition tag, e.g., a recognition tag described below.

In one embodiment, after translation of both nucleic acids, the array is washed to remove unbound proteins and the translation effector. Detection of an address at which the second polypeptide remains bound is indicative of a protein-protein interaction between the first and second polypeptide of that address.

In another embodiment, a third or competing polypeptide can be present during the binding step, e.g., a third encoding nucleic acid sequence lacking a tag can be included at the address.

In yet another embodiment, the stringency or conditions of the binding or washing steps are varied as appropriate to identify interactions at any range of affinity and/or specificity.

Recognition Tags

A variety of recognition tags can be used. For example, an epitope to which an antibody is available can be used as a recognition tag. The tag can be place N or C-terminal to the sequence of interest. The tag is recognized, e.g., directly, or indirectly (e.g., by binding of an antibody).

Green fluorescent protein. Coding regions of interest are taken from the FLEX repository and transferred into fusion vectors encoding either an N- or C-terminal green fluorescent protein (GFP) tag. These vectors have been made, and the backbones are similar to those encoding the poly-histidine and GST tags. The GFP-tagged proteins, the query, are co-transcribed/translated with the immobilized target proteins. Target-query complexes are allowed to form, and unbound protein is washed away. Target-query complexes are then detected by fluorescence spectroscopy (Spectra Max Gemini, Molecular Devices). The environment of a fluorophore has a strong effect on the quantum yield of fluorescence (i.e., the ratio of emitted to absorbed photons) through collisional processes and resonance energy transfer (a radiative process), so the concentration of target-query complexes that gives an acceptable signal-to-noise ratio will have to be determined experimentally.

Fluorescence polarization can be used to detect the recognition tag while circumventing the need for immobilization and wash steps to detect protein complexes. When GFP-tagged query is bound to target, the polarization of the fluorescence of GFP increases due to the reduced mobility of the complex, and this increase in polarization can be measured. Conventional fluorescence spectroscopy and fluorescence polarization methods can be used to detect protein-protein interactions. See, e.g., Garcia-Parajo et al. (2000) Proc. Natl. Acad. Sci. USA 97, 7237-7242.

Enzymatic reporters. Horseradish peroxidase (HRP) or alkaline phosphatase (AP) polypeptide sequences can be used as the recognition tag. The addition of chromogenic substrate and subsequent colorimetric readout allows for the ready detection of the retention of the second polypeptide. Luciferase can be used as a recognition tag as described in U.S. Pat. No. 5,641,641.

ELISA. In another embodiment, the second polypeptide lacks a recognition tag. Instead, an antibody is available that recognizes a small common epitope, e.g., common to all second polypeptides located on the array. Target-query complexes are detected with antibodies using enzyme-linked immunosorbent assay (ELISA) techniques as is routine in the art. This embodiment can be preferable if the second polypeptide species is constant among all the addresses, but the first polypeptide species varies.

MS (Mass Spectroscopy). In yet another embodiment, the recognition tag is a polypeptide sequence whose mass or tryptic profile, when detected by mass spectroscopy, e.g., MALDI-TOF, is indicative of the presence of the second polypeptide. The recognition tag can be a sequence endogenous to the second polypeptide, or an exogenous sequence. Preferably, the MS recognition tag is selected, e.g., using a computer system, to avoid any ambiguity with other potential polypeptide species or tryptic fragments which could be present at each address.

Multipole Coupling Spectroscopy (MCS). MCS can be used to detect interactions at different addresses of the array. MCS is described, e.g., in PCT WO 99/39190. For example, test polypeptides can be synthesized at different addresses of a molecular binding layer (MBL). The MBL can be coupled at each address of the plurality to interface transmission lines or waveguides. A test signal can be propagate to the MBL and a response detected based on the dielectric properties of the MBL as an indication of binding of a query polypeptide to a test polypeptide at an address. Further, a modulation of the test signal or a dielectric relaxation of the MBL can be detected as an indication of binding of a query polypeptide to a test polypeptide at an address.

Exemplary Protein Complexes

The following exemplary protein complexes can be used to verify or optimize methods or to provide convenient positive and negative controls, e.g., using known interactors of various affinities. Such interactors can include: the signaling proteins cdk4-p16, cdk2-p21, E2F4-p130, and the transcription factors Fos-Jun; components of the DRIP complex (vitamin D Receptor Interacting Proteins; Rachez (1999) Nature 398:824 and Rachez (2000) Mol Cell Biol. 20:2718).

Protein-DNA Screens

Transcription factors that bind to specific DNA sequences may be identified. Here DNA is the query molecule and can be fluorescently labeled. Alternatively, the DNA can be biotinylated and detected by HRP coupled to avidin.

Protein-Small Molecule Screens

An array described herein can be used to identify a polypeptide that binds a small molecule. The small molecule can be labeled, e.g., with a fluorescent probe, and contacted to a plurality of addresses on the array (e.g., prior, during, or after translation of the programming nucleic acids). The array can be washed after maintaining the array such that the small molecule can bind to a polypeptide with an affinity tag. The signal at each address of the array can be detected to identify one or more addresses having a polypeptide that binds the small molecule.

Other signal detection methods include surface plasmon resonance (SPR) and fluorescence polarization (FP). Methods for using FP are described, for example, in U.S. Pat. No. 5,800,989. Methods for using SPR are described, for example, in U.S. Pat. No. 5,641,640; and Raether (1988) Surface Plasmons Springer Verlag.

In another embodiment, the invention features a method of identifying a small molecule that disrupts a protein-protein interaction. The array is programmed with a first and a second nucleic acid which respectively encode a first and second polypeptide which interact. The first polypeptide includes an affinity tag and second polypeptide includes a recognition tag. A unique small molecule is contacted to an address of the array (e.g., prior, during, or after translation of the programming nucleic acids). The array can be washed after maintaining the array such that the small molecule, the first and the second polypeptide can interact. The signal at each address of the array is detected to identify one or more addresses having a small molecule that disrupts the protein-protein interaction.

Pre-Clinical Evaluation of Lead Compounds

An application that exploits the ability to screen for small molecule interactions with proteins could be the pre-clinical evaluation of a lead drug candidate. Drug toxicities often result not from the intended activity on the target protein, but some activity on an unrelated binding protein(s). Even when these adventitious binding proteins do not cause toxicity, they can adversely affect the drug's pharmacokinetics. A comprehensive protein array would make the pre-clinical identification of these adventitious binders rapid and straightforward.

Medicinal Chemistry

The small molecule screen could become a rapid and powerful platform by which medicinal chemistry and SAR could be performed. Chemical modifications of small molecules could be tested against the array to see if changes improve specificity. Compounds could be exposed first to hepatic lysates or other metabolic extracts that mimic metabolism in order to create potentially toxic metabolites that can also be screened for secondary targets. Recursion of this process could lead to improved specificity and tighter binding molecules.

Mass Spectroscopy

The polypeptide array can be used in conjunction with mass spectroscopy, e.g., to detect a modified region of the protein. An array is prepared as described herein with due consideration for the flatness, conductivity, registration and alignment, and spot density appropriate for mass spectroscopy.

In one embodiment, the method identifies a polypeptide substrate for a modifying enzyme. Each address is provided with a nucleic acid encoding a unique test polypeptide. Each address of the array is contacted with the modifying enzyme, e.g., a kinase, a methylase, a protease and so forth. The enzyme can be synthesized at the address, e.g., by include a nucleic acid encoding it at the address with the nucleic acid encoding the test sequence. After sufficient incubation to assay the modification step, each address is proteolyzed, e.g., trypsinized. The resulting peptide mixtures can be subject to MALDI-TOF mass spectroscopy analysis. The combination of peptide fragments observed at each address can be compared with the fragments expected for an unmodified protein based on the sequence of nucleic acid deposited at the same address. The use of computer programs (e.g., PAWS) to predict trypsin fragments is routine in the art. Thus, each address of the array can be analyzed by MALDI. Addresses containing modified peptide fragment relative to a predicted pattern or relative to a control array can be identified as containing potential substrates of the modifying enzyme.

The amount of modifying enzyme contacted to an address can be varied, e.g., from array to array, or from address to address.

For example, this approach can be used to identify phosphorylation by comparing the masses of peptide fragments from an address that having a kinase, and an address lacking the kinase. Pandey and Mann (2000) Nature 405:837 describe methods of using mass spectroscopy to identify protein modification sites.

In another embodiment, the modifying enzyme is varied at each address, and the test polypeptide, the polypeptide with the affinity tag for attachment to the substrate, is the same at each address. Both the modifying enzyme and the test polypeptide can be synthesized on the array by translation of encoding nucleic acid sequences. Mass spectroscopy is used to identify an address having a modifying enzyme with specificity for the test polypeptide as enzyme-substrate.

Mass spectroscopy can also be used to detect the binding of a second polypeptide to the target protein. A first nucleic acid encoding a unique target amino acid sequences and an affinity tag is disposed at each address in the array. A pool of nucleic acids encoding candidate amino acid sequence is also disposed at each address of the array. Each address of the array is translated and washed to remove unbound proteins. The proteins that remain bound at each address, presumably by direct interaction with the target proteins, can then be detected and identified by mass spectroscopy.

Assay to Identify Folded Proteins

The NAPPA array can be used to identify appropriately folded protein species, or proteins with appropriate stability. For example, arrays can be provided with a nucleic acid sequence encoding a random amino acid sequence, a designed amino acid sequence, or a mutant amino acid sequence at each address. Such an array can be used to analyze the results of a computer-designed polypeptide, the results of a DNA-shuffling, or combinatorial mutagenesis experiment. The array is contacted with transcription and translation effectors, and subsequently washed provide purified polypeptides at each address.

Subsequently, each address of the array is monitored for a property of the folded species. The property can be particular to the desired polypeptide species. For example, the property can be the ability to bind a substrate. Alternatively, the property can be more general, such as the fluorescence emission profile of the polypeptide when excited at 280 nm. Fluorescence, particularly of tryptophan residues is an indicator of the extent of burial of aromatic groups. Upon denaturation, the center of mass of the fluorescence of exposed tryptophans is shifted. In additional, at an appropriate detection wavelength, the intensity of fluorescence varies with the extent of folding. The array, or selected addresses of the array, can be incrementally exposed to increasing denaturing conditions, e.g., by thermal or chemical denaturation. Thermal denaturation is useful as it does not require altering solutions contacting the array. Thus, if the array contains partitions, subsequent to the washing step, binding of the affinity tag to its handle on the substrate is not required. Addresses showing cooperative folding transitions or increased stability are thus readily identified

Additional properties for monitoring folding include fluorescent detection of ANS binding, and circular dichroism,

Selection Using Display Technologies

In another aspect, the NAPPA platform is used to screen—in a massively parallel format—a first collection of polypeptides for binding to members of a second collection of polypeptides.

The first collection of polypeptides is prepared in a display format, e.g., on a bacteriophage, a cell, or as an nucleic acid-polypeptide fusion (Smith and Petrenko (1997) Chem. Rev. 97:391; Smith (1985) Science 228:1315; Roberts and Szostak (1997) Proc. Natl. Acad. Sci. USA 94:12297). For a review of display technologies see Li (2000) Nat. Biotech. 18:1251. The first collection can be obtained from any source, e.g., a source described herein. In one illustrative example, the first collection is an artificial antibody library.

The second collection of polypeptides is distributed on an array described herein For example, a nucleic acid encoding each polypeptide of the second collection can be disposed at a unique address of the array. The array is prepared as described herein.

Before, during, or after translation of the encoding nucleic acids, the first collection in display format, termed display polypeptides, is applied to the array. After translation of the encoding nucleic acid, the array is washed to remove unbound display polypeptides. Then, presence of a display polypeptide at at least one address is detected, e.g., by amplification of the nucleic acid portion of nucleic acid-polypeptide fusion; by propagation of a cell or bacteriophage displaying the display polypeptide; and so forth.

Extracellular Proteins

In one embodiment, an extracellular polypeptide or extracellular domain can be displayed on a NAPPA array, e.g., by contacting the array with conditions similar to the extracellular, endoplasmic reticulum, or Golgi milieu. For example, the conditions can be oxidizing or can have a redox potential that is optimized for extracellular protein production. The array can be additionally contacted with modifying enzymes found in the secretory pathway, e.g., glycosylases, proteases, and the like.

In another embodiment, the translation effector is applied in conduction with vesicles, e.g., endoplasmic reticular structures. The vesicles can include an affinity tag to anchor the vesicle to the array. In such an embodiment, the encoding nucleic acid need not contain an affinity tag.

An array of extracellular proteins or extracellular protein domains can be used to identify interactions with other extracellular proteins; or alteration of living cells (e.g., the adhesive properties, motility, or the secretory repertoire of a cell contacting the the extracellular protein).

Transmembrane Proteins

Transmembrane proteins can be displayed on a NAPPA array by separately producing the nucleic acids encoding the ecto- or extracellular domains, and the cytoplasmic domains. The extracellular domains and the cytoplasmic domains can be encoded at separate addresses or the same address. Alternatively, only one of the two types of domains is encoded on the array.

In another embodiment, the transmembrane domain can be excised. Ottemann et al.(1997) Proc. Natl. Acad. Sci. USA 94:11201-4 describe a method for excising a transmembrane domain to generate a soluble functional protein.

In yet another embodiment, in vitro translation on the array further includes providing vesicles derived from endoplasmic reticulum.

Contacting Array with Cells

In another embodiment, at least one address of the array, e.g., after translation of encoding amino acids, is contacted with a living cell. After contacting the array, the cell or a cell parameter is monitored. For example, polypeptide growth factors can be arrayed at different addresses, and cells assayed after contact to each address. The cells can be assayed for a change in cell division, apoptosis, gene expression (e.g., by gene expression profiling), morphology changes, differentiation, proteomics analysis (e.g., by 2-D gel electrophoresis and mass spectroscopy), and specific enzymatic activities.

In one embodiment, a test polypeptide of the array can be detached from the substrate of the array, e.g., by proteolytic cleavage at a specific protease site located between the test sequence and the tag.

In another embodiment, the test polypeptide does not have an affinity tag, but is maintained at an address by physical separation from other addresses of the plurality. The translation effector is optionally not washed from the address. Cells are assayed after being maintained at the address as described above.

Cell-Free Assay Platforms

High-throughput, genome-wide screens for protein-protein, protein-nucleic acid, protein-lipid, protein-carbohydrate, and protein-small molecule interactions can be performed on an array described herein. Each address of the array can include a polypeptide encoded by a nucleic acid clone from a repository of full-length genes, e.g., genes stored in a vector that facilitates rapid shuttling by recombinational cloning.

Kits

Kits are convenient collections of components, e.g., reagents that can be supplied to a user in order to efficiently enable the user to practice a method described herein.

Universal Primer Kit. A universal primer kit provides a simple means for amplifying a collection of encoding nucleic acid sequences in a format suitable for disposal on an array. The kit includes a 5′ universal primer and a 3′ universal primer. The kit can further include a substrate, e.g., with an appropriate binding agent attached thereto.

The 5′ primer can include the T7 promoter and a 5′ annealing sequence, whereas the 3′ primer can include a 3′ annealing sequence and sequence encoding an affinity tag. Nucleic acid coding sequences amplified with the 5′ annealing sequence and the 3′ annealing sequence are further amplified with the universal primer set. The products of this amplification are amenable for immediate disposal on the array.

Moreover, asymmetric PCR can be utilized to create an excess of the coding strand. Single-stranded DNA can be deposited on the array and annealed to a T7 promoter nucleic acid capture probe in order to provide a duplex recruitment site for T7 polymerase.

The kit can further include transcription and/or translation effectors, reagents for amplification, and buffers.

Recombinational Cloning Kit. A recombinational cloning kit provides tools for shuttling multiple encoding nucleic acid sequences, preferably en masse, into a vector having suitable regulatory sequences, and affinity tag-encoding sequence for the NAPPA platform. The kit includes a substrate with multiple addresses, each addressing having a binding agent attached to the substrate. The kit also includes a vector having sequences for generating encoding nucleic acid with affinity tags. Once a nucleic acid sequence is cloned into the vector, the nucleic acid of the vector with the insert is suitable for programming the array.

The vector can include a recombination site, e.g., a site-specific recombination site, or a homologous recombination site. Alternatively, the vector can include unique restriction sites, e.g., for 8-bp cutters, in order to facilitate subcloning sequence encoding test polypeptides. These features facilitate the rapid, and parallel construction of multiple coding nucleic acids for programming the array. Thus, a complex array having many unique polypeptide sequences can be easily produced.

For example, a repository of cloned full-length coding sequences of interested flanked by recombination sites is constructed. Multiple sequences in the repository are shuttled into the vector using in vitro site-specific recombination and enhanced selection techniques (see description of Recombinational cloning above, and The Gateways Manual, Invitrogen, Calif.). Robotics and microtiter plates can be used to rapidly producing the multiple coding nucleic acids for programming the array.

The kit can further include a second vector having recombination sites, appropriate regulatory sequences, and a recognition tag, such as a recognition tag described herein. The user can thus shuttle a nucleic acid encoding a sequence of interest into both a vector with an affinity tag, and a vector with a recognition tag. This compatibility facilitates the generation of protein-protein interaction matrices.

A Network Architecture for Providing a NAPPA Array

A user system 14 and a request server 20 are connected by a network 12, e.g., an intranet or an internet. For example, the user system and the request server can be located within a company, the user system in a research department, and the request server in an applications department. Alternatively, the user system 14 can be located within one company, e.g., in a diagnostics division, and the request server 20 can be located in a second company, e.g., a protein microarray provider. The companies can be connected by a network, e.g., by the Internet, a proprietary network, a dial-up connection, a wireless connection, an intermediary, or a customized procurement network. A network within a company can be protected by a firewall 19.

The request server 20 is connected to a database server 22. The database server 22 can contain one or more tables with records to amino acid sequences of polypeptides (e.g., a relational database). For example, each record can contain one or more fields for the following: the amino acid sequence; the location of a nucleic acid clone encoding the nucleic acid in a repository or clone bank; category field; binding ligands of the polypeptide; co-localizing and/or binding polypeptides; links (e.g., hypertext links to other resources); and pricing and quality control information. The database can also contain one or more tables for classes and/or subsets of amino acid sequence. For example, a class can contain entries for amino acid sequences expressed in a particular tissue, correlated with a condition or disease, originating from a species, having homology to a protein family, related to a biological (e.g., physiological or cellular) process, and so forth.

The request server 20 sends to the user 14 one more choices for amino acid sequence to include on a microarray. The choices are provided in a user-friendly format e.g., a hypertext page with forms (e.g., selection boxes). The choices can be hierarchical, e.g., a first list of choices to determine general user needs, and subsequent choices e.g., of a class of amino acid sequence, or of individual amino acid sequences. The choices can also include pre-designed microarrays, as well as individually customized designs. The server can also recommend appropriate negative and positive control amino acid sequence to include depending on previous selections. Alternatively, the system can be voice based, the queries and selections are transmitted across a telecommunications network, e.g., a telephone, a mobile phone, etc.

The user indicates selections, e.g., by clicking on a form provided on a web page. The request server forwards the selections, e.g., the location of nucleic acid encoding a selected amino acid sequence in a clone bank, to a clone bank robot controller. The robot controller 26 mobilizes a robot to access the clone bank and obtain the desired encoding nucleic acid. Optionally, the nucleic acid can be shuttled from a repository vector into an expression vector using recombinational cloning techniques. In another possible implementation, the nucleic acid stored in the repository is already in an appropriate expression vector for nucleic acid programmable protein microarray production. In still another possible implementation, the nucleic acid is amplified with primers which contain the requisite flanking sequence for disposal on the microarray. For example, one or more primers can include a T7 promoter, and/or an affinity tag.

Once obtained, the nucleic acid is provided to an array maker. The array processing server 24 is also interfaced with the request server 20 and the robot controller 26. The nucleic acid is deposited onto one or more array substrates, e.g., using a method described herein. The array production controller selects one or more addresses at which the nucleic acid is deposited, and records the addresses in a table associated with the array being produced. The array production controller can also vary the amount and method of deposition for any particular sample or address. Such variables and additional quality control information is also stored in the table.

For example, if multiple identical arrays are produced in parallel, one or more arrays can be used for a quality control testing. For example, transcription and translation effectors can be contacted to the array at the production facility. The presence of selected or control proteins is verified by contacting the array with specific antibodies for such proteins, and detecting the binding.

Once produced, an array is prepared for shipping, for example, contacted with a preservative solution, dessicated, and/or coated in an emulsion, film, or plastic wrap. The request server 20 interfaces with a courier system 34, e.g., to track shipment and delivery of the array to the user. The request server also notifies the user of the status of the array production and shipment throughout the procurement process, e.g., using electronic mail messages.

The request server interfaces with a business-to-business server to initiate appropriate billing and invoicing as well as to process customer service requests.

Diagnostic Assays

A variety of polypeptide microarrays can be provided for diagnostic purposes. The array can be used as a screening tool to look for antibodies that bind to specific proteins. This could be applied for the generation of monoclonal antibodies in a high-throughput setting or in the context of measuring immune responses in a patient. ELISA techniques can be used for detection.

Antigen Arrays. One class of such arrays is an array of antigens, displayed for the purpose of determining the specificity of antibodies in a subject. The array is programmed such that each address represents a different antigen of a pathogen or of a malady (e.g., antigens significant in allergies; transplant rejection and compatibility testing; and auto-immune disorders).

In one embodiment, the array has antigens from a plurality of bacterial organisms. Computer programs can be optionally used to predict likely antigens encoded by the genome of an organism (Pizza et al. (2000) Science 287:1816). In a preferred embodiment, each address has disposed thereon a unique antigen. In another preferred embodiment, each addresses has a plurality of antigens, all being from the same species. Thus, for example, binding of a subject's antibody to an address indicates that the subject has been exposed to a pathogen represented by the address.

In another preferred embodiment, the array is used to track the progression of complex diseases. For example, diseases with antigenic variation (e.g., malaria, and trypanosomiasis) can be accurately diagnosed and/or monitored by identifying the repertoire of specific antibodies in a subject.

In another embodiment, the array can be used to detect the specific target of an autoimmune antibody. For example, isolated antibodies or serum from a subject having type I diabetes are contacted to an array having islet-cell specific proteins present at different addresses of the array.

Antigen arrays also provide a convenient means of monitoring vaccinations and disease exposure, e.g., in epidemiological studies, veterinary quarantine, and public health policy.

Antibody Arrays. A second class of diagnostic arrays is arrays of antibodies. A variety of methods are available for identifying antibodies. Monoclonal antibodies against a variety of antigens are identified. The nucleic acids encoding such antibodies are sequenced from the genome of hybridoma cells. The nucleic acid sequence is used to engineer single-chain variants of the antibody. Thus, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). The encoding nucleic acid sequence can be recombined into an appropriate vector, e.g., a vector described above with promoter and affinity tag encoding sequences.

In addition, the antibody sequence can be engineered to remove disulfides (Proba K (1998) J Mol. Biol. 275:245-53). Alternatively, after translation and washing of the array, the array is subject to oxidizing conditions, e.g., by contacting with glutathione. The antibodies can be coupled to the array with streptococcal protein G, or S. aureus protein A. Further, specialized antibodies such as modified or CDR-grafted version of naturally occurring antibodies devoid of light chains can be used. The antibodies of camel (e.g., Camelus dromedaries) are naturally devoid of light chains (Hamers-Casterman C (1993) Nature 363:446-8; Desmyter et al. Nat Struct Biol September 1996; 3(9):803-11).

A patient sample can then be contacted to the array. Non-limiting examples of patient samples include serum proteins, proteins extracted from a biopsy obtained from the patient, and so forth. In addition, cells themselves can be contacted to the array in order to query for antigens displayed on the cell surface.

In one embodiment, the sample is modified with a compound prior to being contacted to the array. For example, the sample can be biotinylated. Addresses that bind proteins in the sample are then identified by contacting the array with labeled streptavidin or labeled avidin. In another embodiment, the sample is unlabelled. MALDI, SPR, or another techniques are used to identify if a protein is bound at each address. Arrays can be designed to identify proteins associated with various maladies, e.g., to detect antigens associated with cancer at various stages (for example, early, and pre-metastatic stages) or to provide a prediction (for example, to quantitate the abundance of an antigen correlated with a condition).

Proteins can be used as biomarkers. For example, antigens that are associated with a particular condition can be considered a biomarker. Examples of antigens include CEA, CA-125 and PSA. PSA, for example, can be used to evaluate risk or presence of prostate cancer. Biomarkers can be evaluated, e.g., by contacting a sample from a subject to an array that includes proteins that bind (e.g., specifically bind) to one or more of biomarker proteins. A wide range of analyte specific reagents can be used (e.g., aptamers, antibodies, and minibodies). The array can be an array described herein or prepared by a method described herein. Accordingly, in one aspect, the disclosure features an array that includes a plurality of capture reagents (e.g., analyte specific reagents such as aptamers, antibodies, and minibodies). The array can be used to evaluate a sample, e.g., a sample obtained from a subject.

In addition to detecting protein biomarkers, it is useful to evaluate a subject to detect their antibody or antibody responses. For example, the presence of an antibody can be an indicator of a disorder, e.g., an autoimmune disorder or a neoplastic disorder. Abundance of certain antibodies or biomarkers can be correlated with tumor burden.

Cancer patients may spontaneously produce antibodies against “tumor antigens.” These antigens are frequently proteins that are shed by tumors and that are not encountered by the immune system. Thus, auto-antibodies can be produced against them. These auto-antibodies against tumor antigens may predate clinical cancer presentation by some time or even years. Further, antibodies can persist in circulation despite potential fluctuations of antigen (e.g., diurnal cycles). Antibodies also tend to be more protease resistant and are easily detectable.

Methods for evaluating biomarkers and antibodies have a variety of applications including diagnostics and for monitoring disease progression. The use of multiplexing also enables increased confidence in the result.

An alternative format to using an array of capture reagents is to use a reverse phase protein blot. Multiple samples (e.g., of complex nature, e.g., obtained from multiple different subjects) can be disposed on an array. The samples can also include different fractions of an original sample, e.g., an original sample obtained form a subject.

Another format for analyzing a sample is to resolve the sample into fractions using one or more methods (e.g., chromatography methods such as ion exchange, hydrophobic interaction, and size exclusion; gel resolution, e.g., isoelectric focusing, PAGE). If plural methods are used, the sample can be subject to a first and second dimension. The fractions can be printed onto multiple substrates, e.g., to provide replicate arrays. Samples (e.g., sera), e.g., from patients and optionally controls, can be contacted to the substrate to characterize the patients samples and/or the fractions.

Vaccine Development

The NAPPA arrays provide an improved method for developing a vaccine. One preferred embodiment includes identifying possible antigens for use in a vaccine from the sequenced genome of a pathogen. Pizza et al. (2000) Science 287:1816 describe routine computer-based methods for identifying ORFs which are potentially surface exposed or exported from a pathogenic bacteria. The method further includes making 1) a nucleic acid that serves as a DNA vaccine for expressing each candidate antigen, and 2) a nucleic acid encoding the ORF and an affinity tag in order to program an array. The recombination cloning methods described herein are amenable for generating such a collection of nucleic acids.

The nucleic acids serving as a DNA vaccine can be assembled into multiple random pools and used to immunize a plurality of subjects, e.g., mice. Subsequently, each immunized subject is challenged with the pathogenic organism. Serum is collected from subjects with improved immunity.

An array is provided with a unique encoding nucleic acid at each address. The array is translated and then contacted with the serum from a subject with improved immunity. Binding of a serum antibody to an address are indicative of the address having a polypeptide that is an antigen useful for vaccination against the pathogen.

In another embodiment, a DNA vaccine is substituted with conventional injection of antigens, e.g., as described in Pizza et al., supra.

Network for Diagnostic Assay

A network links health care providers, subjects, and an intermediary server for the purpose of providing results of diagnostic NAPPA arrays. Health care providers can include a primary care physician; and a specialist physician, e.g., infectious disease specialist, rheumatologist, hematologist, oncologist, and so forth; and pathologists. Within a health care institution, such providers can be linked by an internal network attached to an external network by a firewall. Alternatively, the providers can be located on different internal networks that can communicate, e.g., using secure and/or proprietary protocols. The external network can be the Internet or other well-distributed telecommunications network.

The subject can be a human patient, an animal, a forensics sample, or an environmental sample (e.g., from a waste system).

A sample, e.g., of blood, cells, biopsy, serum, or bodily fluid, provided by the subject is delivered to the array diagnostic service, for example by a courier. Tracking provided by the courier system can monitor delivery. The delivered sample is analyzed according to instructions, e.g., accompanying the sample, or provided across the network. The instructions can indicate suspected disorders and/or requested assays.

The array is programmed such that after translation, each address will contain a different antigen or antibody (e.g., as described above). For common diagnostics, NAPPA arrays can be prepared in bulk at the same or another facility.

The sample is optionally processed and then is contacted to a nucleic acid programmable array, e.g., before or after translation to the encoding nucleic acid. Sample handling and detection can be controlled automatically by the array diagnostic server which is interfaced with robotic and detection equipment. The binding of the sample to the array is then detected by the array diagnostic server. Addresses wherein binding of the sample to the array is detected are recorded, e.g., in a table that is store in a database server. An intermediary server is used to transmit results, e.g., securely, back to the health care providers, e.g., the primary care physicians, and the specialist. Optionally, the patient or subject can be directly notified if results are available.

The results can be stored in the database server 58 and/or transmitted to one or more of the physicians, and health-care providers. The results also may be made available e.g., for meta-analysis by public health authorities and epidemiologists.

Informatics

A computer system, containing a repository of observed interaction is also featured. The computer system can be networked to receive data, e.g., raw data or processed data, from a data acquisition apparatus, e.g., a microchip slide scanner, or a fluorescence microscope.

The computer system includes a relational database. The database houses all data from multiple screens, e.g., using different arrays. One table contains table rows for each experiment, e.g., describing the microarray production number, experiment date, experimental conditions, and so forth. The raw data from a GFP-based interaction microarray experiment, for example, is stored in a second table with table rows for each address on the array. The second table has fields for observed fluorescence, background fluorescence, the amino acid sequences present at the microarray address, other annotations, links, cross-references and so forth.

Thus, the database provides a comprehensive catalog of biomolecular interactions. The system is designed to facilitate digital access to the data in order to interface the experimental results with predictive models of interactions. The system can be accessed in real time, e.g., as microarray data is acquired, and from multiple network stations, e.g., multiple users within a company (e.g., using an Intranet), multiple customers of a data provider (e.g., using secure Internet communication protocols), or multiple individuals across the globe (e.g., using the Internet).

Clustering algorithm can be applied to records in the database to identify addresses which are related. See, e.g., Eisen et al. ((1998) Proc. Nat. Acad. USA 95:14863) and Golub et al. ((1999) Science 286:531) for methods of clustering microarray data.

Example

In one embodiment, the following components are used to construct a protein array:
Expression vector—pANT7_cGST and pANT7_nHA which express C-terminal tags GST and an N-terminus HA, respectively. These vectors have a T7 promoter and a ˜500 bp IRES signal which provides optimal expression in rabbit reticulocyte lysate.
Biotin-psoralen conjugate and avidin—To modify the cDNA and the cDNA immobilize on the array.
Aminosilane coated glass slide to bind avidin and capture the cDNA molecules.
Rabbit reticulocyte lysate and T7 polymerase for coupled transcription and translation that produces the target proteins
Anti-tag antibodies (e.g., anti-GST or anti-HA) to detect expressed proteins
Tyramide signal amplification (TSA) system for fluorescent detection.
   Preparation of DNA

Plasmid DNA is grown in 300 mL-500 mL in DH5α bacterial cultures. DNA is purified using standard alkaline lysis protocol from Molecular cloning (Sambrook et al). The prep is then pre-cleared using 96-well filter plates from Qiagen TURBO™ or REAL DNA™ miniprep kits. The DNA is then de-salted using either Millipore plasmid plate or MICRON™ tubes from Amico. Psoralen biotin conjugate (0.11 μg) is added to 100 μL of DNA (˜1-2 mg/mL) in a UV flat bottom plate from Co-Star. The plate is placed on ice and exposed to UV light (365 nm) for 20 min. Upon UV exposure, the sample is extracted twice with two volumes of water saturated butanol. Top layer (organic layer) is discarded and the bottom layer (aqueous) can be used for arraying or stored for future use.

Arraying

A master mix (3 μL) containing avidin (33 mg/mL), anti-GST antibody (1:100 of stock from Amersham Pharmacia) and a NHS ester based linker (2 mM, BS3, Pierce) is added to the biotinylated DNA (20 μL). Array sample is mixed till a white precipitate forms and then briefly spun down (e.g., to remove excess avidin). Currently a GMS427™ arrayer is used to array these samples on a standard amino coated glass slide at 1 mm spacing.

Developing NAPPA

The slides are incubated in a humid chamber at 4° C. overnight. Arrays at this point are stable at room temperature for weeks. The arrays are then blocked with either 5% milk or 1% BSA or SUPERBLOCK™ (Pierce), these blocking solutions are supplemented with 0.2% Tween. Blocking buffer is gently rinsed with de-ionized water, and dried. A hybridization chamber such as a HYBRIWELL™ (Grace Biolabs) is placed on the slide before adding the cell free expression system (100 μL). The slides are incubated at 30° C. for 1.5 hr and then at 15° C. for 30 hrs (the cooling step can be eliminated). The slides are removed and the cell free expression lysate is rinsed with blocking buffer of choice. The slide is further blocked for ˜1 hr in fresh blocking buffer. Primary antibody is added to slide for 1 hr. The slide is rinsed with blocking buffer before secondary antibody (anti-mouse conjugated to horse radish peroxidase HRP) is added to the slide. TSA (100 mL, substrate for HRP) is added to each slide for fluorescence detection. Signals can be detected using standard DNA microarray scanners.

Example

Protein microarrays provide a powerful tool for the study of protein function. This example describes, inter alia, methods of providing protein microarrays by disposing cDNAs onto glass slides and then translating target proteins, e.g., with mammalian reticulocyte lysate. This method can be used to obviate the need to purify proteins, avoid protein stability problems during storage and capture sufficient protein for functional studies. The versatility of this technology was demonstrated in one instance by mapping pairwise interactions among 29 human DNA replication initiation proteins, recapitulating the regulation of Cdt1 binding to select replication proteins, and mapping its geminin binding domain.

In one embodiment, our approach to address these concerns entails programming cell free protein expression extracts with cDNAs to express the proteins at the time of the assay without the need for advanced purification. This strategy substitutes using purified proteins with cDNAs encoding the target proteins at each feature of the array. The proteins are then transcribed/translated by a cell-free system and immobilized in situ using epitope tags fused to the proteins. For example, a simplified version of this was accomplished manually using reticulocyte lysate to express various proteins tagged with GST in a microtiter plate coated with anti-GST antibody, but is applicable to other formats, e.g., glass slides. This approach eliminates the need to express and purify proteins separately and produces proteins “just-in-time” for the assay, abrogating concerns about protein stability during storage. This chemistry also has the advantage that mammalian proteins can be expressed in a mammalian milieu, providing access to vast collections of cloned cDNAs.

We developed a version that included several additional features. First, a high density format that minimized the use of cell free extract would allow the simultaneous examination of many proteins at a lower cost per protein. Second, we wished to use a readily available matrix (such as standard glass microscope slides) that did not require specially micro-machined wells and which utilized the widely accessible existing technology for printing and reading DNA microarrays. This design would avoid the need to create specialized equipment to produce and print the arrays and would therefore ensure broad accessibility of the technology.

The array also was designed to provide sufficient protein at each spot to study function, despite more than a 1000 fold reduction in sample volume relative to a microtitre well. The second was identifying an efficient printing chemistry for DNA on glass microscope slides that supported transcription/translation in situ. In addition, once translated, this chemistry had to display rapid, efficient and specific protein capture, without high background signal and without spot-to-spot diffusion or crosstalk.

Printing chemistry

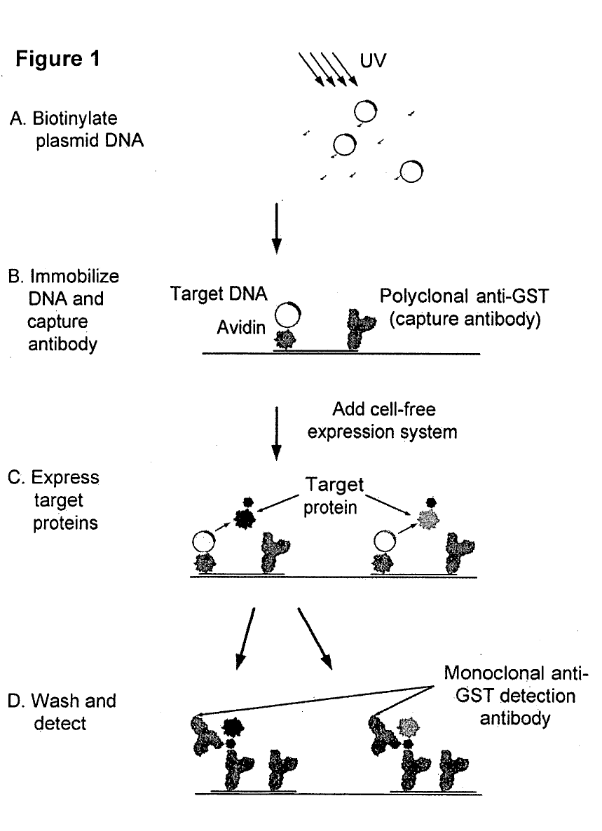

Printing methodology can be selected to balance efficiency of DNA binding and maintenance of a conformation that supported efficient transcription/translation. One efficient strategy included coupling a psoralen-biotin conjugate to the expression plasmid DNA using UV light, and then capturing the modified plasmid DNA on the surface by avidin (FIG. 1).

The addition of a C-terminal GST tag to each protein enabled its capture to the array through an anti-GST antibody printed simultaneously with the expression plasmid in a 15 fold molar excess over the DNA. Other protein fusion tags and capture molecules can be substituted easily for the GST fusion and anti-GST antibodies used here (data not shown). Other useful molar ratios of DNA to binding agent (e.g., antibody) include at least 1:5, 1:10, 1:50, 1:100, 1:200, 1:500, and 1:1000, e.g., between 1:5-1:250. The resulting array was dried and stored at room temperature.

To activate and use the array, a cell-free, coupled transcription/translation system (such as reticulocyte lysate containing T7 polymerase) was added as a single continuous layer covering the arrayed cDNAs on the microscope slide. This unitary application enabled array production without a separation barrier between the features of the array while delivering the expression system. (If desired, one may still use such barriers, e.g., between different sets of addresses, or between each address).

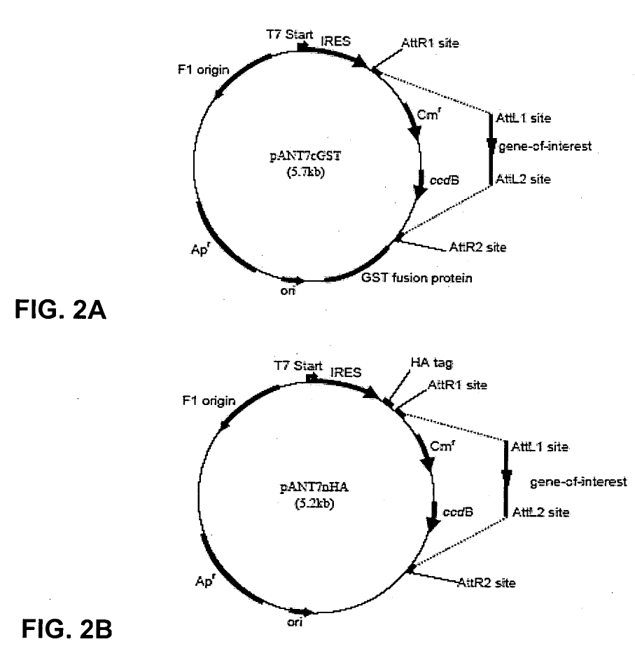

Once printing and expression conditions were established, we tested them on a small set of genes. Expression plasmids encoding eight genes were immobilized onto an array at a density of 512 spots per slide (900 μm spacing). Expression of target protein was confirmed using anti-GST antibody (different from the capture GST antibody) and the signals were measured using a standard glass slide DNA-microarray scanner (FIG. 2a).

Exemplary biotinylation of plasmid DNA and exemplary expression protocol includes: Biotinylation-Psoralen-Biotin (AMBION) is added to DNA at 1:1000 (w/w) and crosslinked with UV (365 nm) for 20 mins. Excess biotin was extracted using 2 vol of water saturated butanol. Expression—Samples were prepared in a 384-well plate (GENETIX) and arrayed using a AFFYMETRIX 427™ arrayer at 60% humidity. Arrayed slides were incubated at 4° C. overnight, blocked with 5% milk (0.2% Tween®-20) prior to expression. Rabbit reticulolysate (100 μL) was added to the slide pre-fitted with a HYBRIWELL™ (GRACE BIOLABS). Expression and immobilization was carried out at 30° C. for 1.5 hr followed by 15° C. incubation for 2 hrs in a chilling incubator (Torrey Pines). Slides were blocked for 1 hr with 5% milk (0.2% TWEEN20) before treatment with primary antibody. Primary antibody for detection of target proteins was anti-GST (Cell Signaling Technologies), and for detection of query proteins was anti-HA (12CA5). Slides were then treated with secondary antibody, anti-mouse conjugated to HRP (Amersham), and developed using Tyramide Signal Amplification system (TSA, PerkinElmer). Developed slides were imaged using a ScanArray 5000XL and quantitated using SCANALYZE™.

We observed an easily detectable signal for all proteins (average S/N ratio=53±14), demonstrating that 100 μL of reticulocyte lysate is sufficient to support protein expression in all 512 spots of the array simultaneously. Signal-to-noise ratio (S:N) and Coefficient of Variation (CV), S/N—Signal is the measured spot intensity, minus the average of the background spots; noise is 1.65 times the standard deviation of the background spots; and the background spots are locations within the same grid that were not printed. CV—Corrected signals for 64 spots for each of 8 proteins were averaged; the average of the 8 means is 4763 and the standard deviation of the 8 means is 1141, for a coefficient of variation of 24%.

There was modest variation in protein expression from gene to gene (Coefficient of Variation=˜24%), but these variations can often be corrected by adjusting the amount of printed plasmid template. By comparing signal intensities to control spots containing purified GST, we estimated that approximately 10 femtomoles (˜675 pg) of protein are produced and captured at each spot.

To verify that the detected proteins were the expected target proteins, and to confirm that there was no crosstalk across the slide, we used target protein-specific antibodies. As expected, anti-Jun and anti-p21 antibodies detected the relevant proteins in the predicted locations, with no detectable diffusion between spots.

Protein-protein interactions. A powerful and straightforward application of NAPPA is the detection of protein-protein interactions. In this application, both the target proteins (affixed to the array by a tag) and the query protein (lacking a tag that interacts with the array) can be transcribed and translated in the same extract. The query protein, in this case Jun, was tagged with an HA epitope and co-expressed with the target proteins. The interaction was visualized using an anti-HA antibody which revealed Jun query protein bound to the Fos target (K_{d}˜50 nM, J. R. Newman, A. E. Keating, Science 300, 2097-101 (Jun 27, 2003). To determine if the binding selectivity observed resembled that observed in biochemical settings, we tested the Cdk inhibitor p16, which is known to bind selectively to Cdk4 and Cdk6 but not the closely related Cdk2.

Application of NAPPA to a Biological System

To further evaluate an implementation of NAPPA in a well-studied biological system, we mapped binary interactions among proteins that participate in the initiation of human DNA replication. This system includes a moderate number of known proteins that form partially characterized complexes including known interactions that acted as positive controls.

Experiments in yeast, Xenopus, and human cells have led to a detailed model for the initiation of eukaryotic DNA replication. Origins of replication are “licensed” in the G1 phase of the cell cycle when the Origin Replication Complex (ORC) recruits the initiation factors, Cdt1 and Cdc6, as well as the mini chromosome maintenance complex (MCM2-7). Together, these factors comprise the pre-replication complex (pre-RC). In S phase, the pre-RC is converted into an active replication fork by the protein kinases Cdc7 and Cdk2, a process that involves origin binding of at least two additional initiation factors, MCM10 and Cdc45 leading to DNA synthesis.

We cloned and sequence verified 29 human genes involved in DNA replication initiation and recombined them into the target and query expression vectors. All 29 target DNAs (plus Fos and Jun as positive controls) were immobilized and expressed in a microarray format. Each gene was expressed in duplicate, and showed high reproducibility between the duplicates. Signals were readily detected for all of the target proteins, ranging from 270 pg (4 fmols) to 2600 pg (29 fmols), a seven-fold range that falls well within the range observed in protein-spotting protein microarrays (10 pg-950 pg, H. Zhu et al., Science 293, 2101-2105 (Sep. 14, 2001)). Included were also two protein registration markers, whole mouse IgG to monitor slide-to-slide variation and purified recombinant GST to assess target protein expression. Each of the query proteins was used to probe a pair of duplicate arrays to generate a 29×29 protein interaction matrix.

We found 110 interactions among the proteins in the replication complex, averaging 7.7 interactions per protein (range 3-16). We detected 47 interactions previously identified in our literature survey, and 63 apparently novel interactions. We compared these results to known interactions that had been demonstrated biochemically using purified proteins. We detected 17 out of the 20 such interactions corresponding to a success rate of 85%; we did not detect interactions between cyclin A1-Cdk2, Cdt1-MCM6, and ORC2-ORC3. We also detected 19 of the 36 interactions (53%) that have been reported based upon co-immunoprecipitation (IP). Because this implementation of NAPPA was designed only to detect binary interactions, it is expected to overlook some interactions detected by IP, which may be indirect and include interactions mediated by bridging proteins. These latter interactions would be suggested by a network in which two proteins shared a common binding partner. Indeed, we could identify a common binding protein for each of the 17 IP interactions not detected by the method. Some of the interactions were detected in only one query-target direction, which may reflect potential steric effects of the GST and/or HA tags.

The human replication complex interaction map. A variety of biochemical experiments have identified two stable complexes, ORC and MCM2-7, in the pre-RC of many species including yeast, Xenopus, Drosophila, mouse and human. Consistent with this, the microarray experiments detected many interactions (28% of all detected interactions) within and between these two complexes. We have identified 10 unique interactions among the six ORC subunits, consistent with a stable complex, and in agreement with the current ORC model. Similarly we observed most known interactions within the MCM complex except those involving MCM6, which was among the proteins evidencing low expression as both target and query.

The contact points among Cdc6, Cdt1 and the ORC proteins required for pre-RC formation are not well understood. Here we find that Cdc6 interacts directly with all of the ORC proteins except ORC4 and that Cdt1 interacts specifically with ORC1 and ORC2.

In S phase, the loading of Cdc45 to the chromatin is postulated to activate the helicase activity of the bound MCM2-7 complex. Interestingly, we did not observe any direct interactions between Cdc45 and the MCM2-7 proteins. Cdc45 interacted with MCM10 which in turn interacted with several MCM2-7 proteins, suggesting that MCM10 could act to recruit Cdc45 to the MCM2-7 complex. Recent experiments showed that MCM10 is indeed required for Cdc45 binding to chromatin; however, it is not clear if this effect involved direct interaction between Cdc45 and MCM10, suggesting the need for further experiments. Still other experiments can include translation of factors encoding enzymes, e.g., CDK-cyclin complexes.

Functional Studies on a Microarray Format

Cdc6 and Cdt1 are both necessary to recruit the MCM2-7 complex onto chromatin. We detected many interactions among these proteins but none between Cdt1 and the MCM2-7 proteins, although they co-immunoprecipitate. We noted that Cdt1 and MCM2 both share Cdc6 as a binding partner, suggesting that Cdc6 could bridge Cdt1 to the MCM2-7 complex. The open format of NAPPA supports the expression of proteins in addition to the target and query, allowing the examination of multi-protein complexes and their regulation. By exploiting this feature, we demonstrated MCM2 binding to Cdt1 only in the presence of co-expressed Cdc6, but not in its absence. Thus, it is likely that Cdc6 acts as a bridging protein, although enzymatic or allosteric effects cannot be ruled out. In any case, this experiment illustrates that regulatory interactions can be detected by the protein microarray format.

To further examine Cdt1 protein function, we focused on its interaction with geminin. Geminin is thought to bind to Cdt1 in the S and G2 phases to prevent the re-loading of the MCM complex onto origins of DNA replication that have already fired. Previous work had suggested that geminin binds somewhere within a relatively large domain of Cdt1 (177-380 aa). Given the importance of the geminin-Cdt1 interaction, we chose to map more precisely the binding domain of geminin on human Cdt1 using NAPPA. This was accomplished by generating a series of end deletion fragments of Cdt1, recombining them into pANT7_cGST, expressing the partial length proteins on the array and probing the array with HA-geminin as query protein. Using this approach we localized a ˜14aa sequence (198-212aa) that was necessary for binding.

We then tested a 77 amino acid fragment (135aa-212aa) containing this sequence and demonstrated that it was sufficient for geminin binding, albeit somewhat more weakly. We have mapped the geminin binding domain on Cdt1 to include a core 14 amino acid sequence (198-212aa) and demonstrated that a short polypeptide containing this domain is sufficient for binding.

The use of NAPPA offers a number of advantages in this regard. This method obviates the need to express and purify the proteins separately, offering great versatility in creating arrays. Designing a new array is as simple as selecting a new set of cDNAs to print. Moreover, proteins can be expressed in their natural milieu, such as expressing mammalian proteins in a reticulocyte lysate. Lastly, the synthesis of target proteins “just-in-time” for the assay allows them to remain continuously in an aqueous state avoiding denaturation.

The printing chemistry described here extends the application of in vitro synthesis of proteins from a macroscopic tool to one that can be executed at high density on a standard microscope slide. The resulting arrays can achieve much greater throughput, be stored dry at room temperature for weeks without loss of signal, and the reagent costs are minimal.

To evaluate this implementation NAPPA, we have verified several canonical protein-protein interactions, including Fos-Jun, and Cdks with the appropriate cyclins. When we performed a 29×29 NAPPA interaction matrix using a set of 29 known eukaryotic replication initiation factors, we identified 110 interactions. The results here compare favorably to other protein interaction methods.

Note that NAPPA can be readily adapted to assess the binding selectivity of small molecules to a family of related proteins (e.g., kinases) or to a mutant series of a single protein, to screen for immune responses to a large panel of antigens, or to screen for substrates for an active enzyme. The increasing availability of large repositories of protein-expression ready cDNA clones in recombinational vectors will provide a rich content source that will amplify the power of this technique to study protein function.

### FIGURES

FIG. 1: Exemplary NAPPA chemistry. (A) Biotinylation of DNA. Plasmid DNA is crosslinked to a psoralen-biotin conjugate using UV light. (B) Printing the array. Avidin (1.5 mg/mL, Cortex), polyclonal GST antibody (Amersham, 50 μg/mL) and Bis(sulfosuccinimidyl) suberate (2 mM, Pierce) are added to the biotinylated plasmid DNA. Samples are arrayed onto glass slide treated with 2% 3-aminopropyltriethoxysilane (Pierce) and 2 mM dimethyl suberimidate.2HCl (Pierce). (C) In situ expression and immobilization. Microarrays were incubated with 100 μL per slide rabbit reticulocyte lysate with T7 polymerase (Promega) at 30° C. for 1.5 hr then 15° C. for 2 hrs in a programmable chilling incubator (Torrey Pines). (D) Detection. Target proteins are expressed with a C-terminal GST tag and immobilized by the polyclonal GST antibody. All target proteins are detected using a monoclonal anti-GST antibody (Cell Signaling Technology) against the C-terminal tag ensuring detection of full length protein.

Expression of target proteins on a NAPPA microarray format. (A) 8 target plasmid DNAs encoding C-terminal GST fusion proteins in pANT7_cGST were immobilized onto the glass slide at a density of 512 spots per slide (900 um spacing). The target proteins were expressed with 100 μL rabbit reticulocyte lysate supplemented with T7 polymerase. Signals were detected using anti-GST antibody and TSA reagent (PerkinElmer). To cross-evaluate, (B) Jun and (C) p21 were also detected using protein specific antibodies. The 8 genes were queried for potential interactors with D) Jun and E) p16. Query DNA encoding an N-terminal HA tag was added to the reticulocyte lysate prior to expressing the target proteins. Target and query proteins were co-expressed and detected with an anti-HA antibody (12CA5). The bar graphs in D-E show average intensity (±S.D.) from 64 samples for each interaction. Images were quantified using SCANALYZE™ software. The signals were corrected for local background.

Expression of human DNA replication proteins (A) Target DNAs representing 29 human DNA replication proteins and 2 positive controls were immobilized and expressed on the array in duplicate. Expression of all target proteins was confirmed by anti-GST antibody (left panel). Two protein registration markers, purified recombinant GST (22 μg/ml, Sigma) and whole mouse IgG (550 μg/mL, Pierce), were also printed as registration spots and to monitor protein expression and slide variation (inset, bottom). (B) Replicate slides from (A) were probed with each member of the DNA replication proteins expressed as HA-tagged query proteins, repeating each query protein on two slides. Slides were probed with (i) HA-Fos, (ii) HA-ORC3 and (iii) HA-MCM2. Interactions were detected using anti-HA antibody and quantified using SCANALYZE™. The signal was calculated by subtracting local background and then standardized using the intensity of whole mouse IgG registration marker. Interactions were considered positive when the signal was greater than 3 times the standard deviation of the background for all instances of the interaction. Interaction map (C) Interactions among the ORC and MCM complex are shown in blue (lines+oval) and green (lines+oval) respectively. Inter-complex interactions are shown in blue-green. Interactions with proteins involved in the formation of pre-RC and pre-IC are shown in red while additional regulatory proteins are shown in brown. All other interactions are shown in orange. The arrows of the connector show the direction (from target to query) of the interaction and the weight given to the connector depicts the strength of the signal.

Characterization of Cdt1. (A) Cdt1 interactions. Interactions among Cdt1, Cdc6, Geminin and the MCM proteins as demonstrated by NAPPA. Interactions in red were used, to study the regulation of Cdt1 binding to the MCM complex. Cdt1 regulation. Target proteins Cdc45, MCM5 and Cdt1 were expressed in duplicate and confirmed by anti-GST antibody. The target proteins were probed with either HA-MCM2 alone (left panel) or in the presence of co-expressed His-Cdc6. The binding of MCM2 was detected using an anti-HA antibody. Cdt1 deletion mapping. Fragments from various regions of Cdt1 were generated by PCR and cloned into target expression vectors. The partial or full-length polypeptides were expressed and detected on the array using anti-GST antibody. To identify the binding region of geminin, the array was queried with HA-geminin and developed using anti-HA antibody. To show sufficient binding a Cdt1 deletion fragment (132aa-212aa) was expressed along with full length Cdt1, which was again queried with geminin.

NAPPA concept in a macroscopic format. Microtiter wells coated with α-GST antibody contained cell free expression mix (T7 coupled rabbit reticulolysate) and a plasmid, pANT7_cGST to express a target protein with a C-terminus GST fusion. Each row is programmed to express a different target protein which is then immobilized in the α-GST coated wells. After removing the unbound proteins, each column is treated with a protein-specific antibody to confirm that the target proteins have been expressed and captured.

Optimization of NAPPA chemistry. Plasmid DNA expressing Jun-GST was used as a control to optimize for arraying conditions. The amount of biotin (0-1:300 Biotin:DNA), length of UV exposure (0-60 minutes) and the amount of avidin (0-4.5 mg/mL) were varied to optimize the conditions required to immobilize and express the plasmid DNA. Amount of DNA immobilized on the array was determined by treating the slide for 5 minutes with PicoGreen (1:600, Molecular Probes), and visualized using a microarray scanner. Target protein expression was detected using a monoclonal GST antibody and a secondary anti-mouse antibody conjugated to HRP. The images were developed using chemiluminescent reagent (ECL, Pierce).

Vector maps of expression plasmids. Plasmids used to express the (i) target protein with a C-terminal GST fusion, pANT7_cGST (FIG. 2A), and (ii) query protein with a N-terminal HA tag, pANT7_Nha (FIG. 2B).

Example

Protein arrays can be made in a miniaturized format for displaying hundreds or thousands of purified proteins in close spatial density that provide a powerful platform for the high throughput assay of protein function.

One implementation for producing protein arrays includes spotting plasmid DNA encoding proteins onto an array. The plasmid DNAs are then transcribed and translated by a cell-free system. The expressed proteins are captured and oriented at the site of expression by a capture reagent that targets a tag incorporated into the protein by the plasmid DNA construct. The tag can be either at the N- or C-terminus of the protein or located internally. Instead of a tag, a capture reagent that recognizes some other feature of the encoded proteins can also be used.

Protein arrays permit many biochemical activities to be studied simultaneously. Such arrays can be used to identifying interact proteins, examine the selectivity of drug binding, find substrates for active enzymes and detect for unintended drug interactions. In some implementations, the array is probed with a labeled query molecule to identify interactions with proteins on the array. For example, a labeled candidate kinase inhibitor might be used to screen an array of kinases to determine the affinity of the inhibitor for the different kinases. Such an evaluation can indicate the specificity and preferences of the inhibitor.

Many factors are relevant for protein array production. Some include:

Availability of array content Protein arrays can be produced from collections of cDNAs in protein expression-ready formats. The methods described herein obviate the need to individually produce and purify each protein.

In some embodiments, the proteins are translated in an extract that is from the same species, order, or phylum as the origin of the protein itself. For example, if most proteins on the array are mammalian, a mammalian extract can be used.

The use of the protein translation enables the array to be prepared by disposing nucleic acids at one stage and then to be stored. Translation can then be performed at a later stage, thereby avoiding issues of protein instability and degradation during the storage period. Once translated, the protein array can be used shortly thereafter.

Array surface chemistry. Factors to consider include:

Generality of binding—Ability to bind all proteins that will be spotted on the array.

Binding capacity—Maximum amount of protein captured per feature.

Efficiency of capture—Fraction of spotted protein that is captured on the array.

Orientation—specific vs. random orientation—Proteins can be immobilized either in an orientation specific manner (e.g., by binding via either an N-terminus or a C-terminus tag) or in random orientations (e.g., by chemical attachment at a variety of positions).

Distance from surface—Some attachment methods allow for a spacer (e.g., a large polypeptide tag) that separates the protein from the array surface; other methods (e.g., chemical attachment) bring the proteins in direct contact with the array surface. Increasing the distance between the protein and the array surface reduce any residual steric hindrance caused by the surface and increase accessibility to the protein.

Native or denatured protein—Surface chemistry can be formulated to contain hydrophobic or hydrophilic residues. Given that many proteins have a hydrophilic exterior and a hydrophobic interior, the choice of the surface chemistry could support the binding of non-denatured or denatured protein. (Mrksich, M., and Whitesides, G. M. 1996. Annu Rev Biophys Biomol Struct 25:55-78.)

To circumvent the need to express, purify and spot the protein, this approach prints the plasmids bearing the genes on the array and the proteins are synthesized in situ. The genes are configured such that each encoded protein contains a polypeptide tag used to capture the protein to the array surface. The proteins are expressed using a cell free transcription/translation extract, which can be selected to match the source of the genes (e.g., rabbit reticulocyte lysate for mammalian genes), thus enabling the proteins to be expressed in a more native milieu. The use of appropriate cell-free extracts helps to encourage natural folding and, at least in the case of reticulocyte lysate, is highly successful at expressing most proteins. In addition, some natural post-translational modifications occur in these extracts and/or can be induced by using supplemented lysates. (Starr, C., and Hanover, J. 1990. J Biol Chem. 265:6868-6873.; Walter, P., and Blobel, G. 1983. Methods Enzymol. 96:84-93.)

Arranging the genes so that each has an appropriate capture tag is facilitated by using vectors with recombinational cloning sites. Coding regions inserted in recombinational cloning systems, such as the Invitrogen GATEWAY™ system or Clontech CREATOR™ system, can be readily moved into expression vectors that append the appropriate tag(s) to the coding regions. The transfer reactions themselves are simple, highly efficient, error free and automatable. The assembly of large collections of genes in these systems is currently in progress. (Braun, et al. 2002. Proc Natl Acad Sci USA 99:2654-2659.)

A significant advantage of this embodiment of the NAPPA approach is that it avoids concerns about protein stability. Proteins on the array are not produced until the array is ready for use in experiments; that is, they are made just-in-time. Prior to activation with the cell free transcription/translation extract, the arrays are stable and can be stored dry on the bench for months.

Using this approach in a recent study, 30 human DNA replication proteins were expressed and captured on NAPPA microarrays. The yield of captured protein was 400-2700 pg/feature, which was 1000 fold more than some protein spotting arrays that have 10-950 fg/feature (Zhu, et al. 2001. Science 293:2101-2105). Arrays were used to determine protein-protein interactions (recapitulating 85% of the previously known interactions), to map protein interaction domains by using partial-length proteins, and to assemble multi-protein complexes.

### 2. MATERIALS

Equipment that can be used: Arrayer with solid pins, humidity control; Microarray scanner; Programmable chilling incubator; SpeedVac; Centrifuge: Sorvall RC12, Eppendorf 5417C, IEC Centra GP8; UV light, UVP UVLMS-38, set at 365 nm

2.1. Preparation of the Slides

1. Glass slides (VWR 48311-702).

2. Solution of 2% aminosilane (Pierce 80370) in acetone. Make up 300 mL just before use.

3. Stainless steel 30-slide rack (Wheaton), handle removed.

4. Glass staining box (Wheaton).

5. LOCK & LOCK™ 1.5 cup boxes (Heritage Mint Ltd., ZHPL810).

6. Prepare a 50 mM Dimethyl Suberimidate.2 HCl (DMS) stock solution: 1 g of DMS linker (Pierce 20700) in 40 mL DMSO. Store at −20° C.

7. To coat slides with linker only (for implementations in which avidin/streptavidin is disposed on the array with plasmid DNA and anti-GST antibody): 2 mM DMS in PBS, pH 9.5.

OR

8. To coat slides with avidin/streptavidin (for implementations in which plasmid DNA and anti-GST antibody is disposed on the array without avidin/streptavidin): 2 mM DMS, plus avidin (Cortex CE0101) at 1 mg/mL or streptavidin (Cortex CE0301) at 3.5 mg/mL, in PBS, pH 9.5. For either material 7 or 8, generally make fresh at the time of coating otherwise the DMS linker may hydrolyze over time.

9. Coverslips (VWR 48393-081).

10. Bioassay dishes with dividers (Genetix x6027).

2.2. DNA Preparation

1. The plasmid DNA is prepared in 300 mL cultures grown usually in Terrific Broth media. The DNA preparation is derived from Sambrook, J., Fritsch, E. F., and Maniatis, T. 1989. Molecular Cloning. A laboratory manual. and is summarized below.

2. Prepare Solution 1 (GTE): 50 mM Glucose, 25 mM Tris pH 8.0, 10 mM EDTA (8.0), and 0.1 mg/mL RNAse. Store at 4° C.

3. Prepare Solution 2: 0.2 N NaOH with 1% SDS.

4. Prepare Solution 3: 3M KOAC; add glacial acetic acid until pH is 5.5.

5. 250 mL conical Coming centrifuge bottle.

6. Glass fiber 0.7 micron filter plate, long drip (Innovative Microplate F20060). 7. 96-well deepwell block (Marsh AB-0661).

2.3. Preparation of Samples and Arraying 1. Plasmid DNA (prepared above in 2.2) 2. MICROCON™ YM-100 (100 kDa) tube (Millipore), or DNA binding plate: 100 kDa 96-well filter plate (Millipore plasmid plate).

3. BRIGHTSTAR™ Psoralen-biotin kit (Ambion 1480). Just before use, prepare psoralen-biotin: dissolve the contents (4.17 ng) of the kit in 50 μL DMF (also in kit).

OR

4. EZ-LINK™ Psoralen-PEO-Biotin (Pierce 29986). Prepare stock solution of 5 mg/mL in water and store at −20° C.

5. UV-transparent 96-well plate (Coming 3635).

6. SEPHADEX™ G50 (Sigma-Aldrich).

7. 1.2 μm glass fiber filter plate, long drip (Innovative Microplate F20021).

8. Collection plate, round bottom (Coming 3795).

9. 384 well plate for arraying (Genetix x7020).

10. Polyclonal anti-GST antibody (Amersham Biosciences 27457701).

11. Purified GST protein (Sigma G5663). Prepare stock solution of 0.03 mg/mL in PBS.

12. Whole mouse IgG antibody (Pierce 31204). Prepare stock solution of 0.5 mg/mL in PBS.

13. BS3 (Bis[sulfosuccinimidyl] suberate) linker (Pierce 21580).

14. Bioassay dish dividers to be used as slide racks (GENETIX™ x6027) and deeper bioassay dishes (e.g. CORNING 431111 or 431272; do not use “low profile” dishes).

2.4. Expression of Proteins

1. HYBRIWELL™ gaskets (GRACE BIO-LABS HBW75).

2. Cell free expression system (Rabbit reticulocyte lysate) (PROMEGA L4610).

3. RNASEOUT™ (Invitrogen 10777-019).

4. SUPERBLOCK™ blocking solution in TBS (Pierce 37535).

5. Milk blocking solution: 5% Milk in PBS with 0.2% TWEEN®-20 (Sigma).

2.5. Detection and Analysis

1. Primary AB solution: mouse anti-GST (Cell Signaling 2624) 1:200 in SUPERBLOCK™ (Pierce 37535). Store at 4° C.

2. Primary AB solution: mouse anti-HA (Cocalico) 1:1000 in SUPERBLOCK™. Store at 4° C.

3. Secondary AB solution: HRP-conjugated anti-mouse (Amersham NA931) 1:200 in SUPERBLOCK™. Store at 4° C.

4. Tyramide Signal Amplification (TSA) stock solution: use TSA reagent (PerkinElmer SAT704B001EA). Prepare per kit directions. Keep this solution at 4° C.

5. Milk blocking solution: 5% Milk in PBS with 0.2% Tween20 (Sigma).

6. Coverslips (VWR 48393-081). 7. PicoGreen (Molecular Probes P11495) stock solution: to the 100 μL/vial that comes, add 200 μL TE buffer. Before use do a 1:600 dilution in SUPERB LOCK™.

### 3. METHODS

These examples include efficient immobilization of plasmid DNA onto a solid surface without compromise to integrity. Proteins translated from the plasmid DNA are rapidly captured. In order to immobilize the plasmid, we use a psoralen-biotin bis-functional linker that attaches to the plasmid DNA. Under long wave UV (365 nm), psoralen intercalates into the DNA, creating a biotinylated plasmid. The reaction is robust over a wide range of pH and salt concentrations. The biotinylated plasmid is tethered to the array surface by high-affinity binding to either avidin or streptavidin. In addition to the plasmids, target protein capture molecules are also immobilized on the slide.

In one implementation, plasmids are constructed to express target proteins with a C-terminal glutathione-S-transferase (GST) protein. A polyclonal anti-GST antibody is bound to the array as the capture molecule to immobilize the expressed fusions of target proteins. The presence of the C-terminal fusion tag can later be confirmed by incubating the slides with an antibody that recognizes a different epitope on the tag than the antibody used for capture. The presence of the C-terminal tag indicates that the full-length protein was expressed.

To make this chemistry robust and reproducible, we have used high affinity capture reagents that are well characterized and stable throughout arraying and storage. Moreover, the schemes outlined above can be altered by the user to accommodate different immobilization chemistries and attachment methods for the plasmid DNA and/or target proteins.

3.1. Preparation of the Slides

1. Prepare 300 mL of aminosilane coating solution (2% aminosilane reagent in acetone).

2. Put slides in metal rack (30-slide Wheaton rack).

3. Treat glass slides in the aminosilane coating solution, ˜1-15 min in glass staining box on shaker. Rinse with acetone in rack using wash bottle. Briefly rinse with MILLIQ™ water. Spin dry in SPEEDVAC™ or dry using 0.2 μm filtered air cans or use house air with 2×0.25 μm filters. It is important to use clean air to dry slides in order to prevent contaminating debris from binding to the surface.

4. Store at room temperature in metal rack in LOCK & LOCK™ box.

5. Just before use, prepare linker solution as per instructions on 2.1.7 or 2.1.8 depending on array strategy.

6. Set slides on divider in bioassay dish, with water in the bottom of the tray. Treat each slide with 150-200 μL linker solution and coverslip. Incubate for 2-4 hours at room temperature or overnight in coldroom.

7. Wash with MILLIQ™ water.

8. Put slides in metal rack. Spin dry in SPEEDVAC™.

9. Store at room temperature in metal rack in LOCK & LOCK™ box.

3.2. DNA Preparation

1. Grow 300 mL culture: in a 2 L culture flask, make a 300 mL culture of TB with 10% KPI. Add 300 μL 100 mg/mL ampicillin stock solution. Add 0.5 μL glycerol stock. Put it on a shaker for 16-24 hours at 37° C., 300 rpm.

2. Pellet in 450 mL centrifuge bottle: spin 15 min at 4000 rpm (Sorvall RC12).

3. Add 30 mL of solution 1 and resuspend.

4. Add 60 mL of solution 2 and swirl, no more than 5 minutes.

5. Add 45 mL of solution 3 and shake briefly.

6. Spin at 4700 rpm 15 min.

7. Pass through cheesecloth into 250 mL conical Corning centrifuge bottles.

8. Add 75 mL of isopropanol and shake.

9. Spin at 4700 rpm 15 min (Sorvall RC12).

10. Pour off supernatant.

11. Dissolve pellet in 2 mL in Tris-EDTA buffer (pH 8) and transfer to a 2 mL microfuge tube. Plasmid DNA yield from this preparation is ˜0.5-1.5 μg/μL.

12. Add 200-250 μL to each well of the long drip glass fiber 0.7 micron filter plate (F20060). Stack on top of a deepwell block.

13. Spin at 2000 rpm 20 minutes (IEC Centra GP8).

14. Store the filtrate in the deepwell block at −20° C., or in individual microfuge tubes.

3.3. Preparation of Samples and Arraying 1. Either spin 200 μL of DNA (0.5-1.5 μg/μL) in a MICROCON 100 kDa tube at 1000 g for 20 minutes, or spin 200 μL of DNA in a 100 kDa 96-well filter plate, stacked on top of a discard plate, for 20 minutes at 2000 rpm (EPPENDORF 5417C).

2. Resuspend in 100 μL water. DNA concentration should be 1-2 μg/μL. The goal is to achieve 100 μL of roughly 1 μg/μL of plasmid DNA. This is because the following UV exposure conditions for biotinylation of the plasmid have been optimized for a 100 μL volume. Increasing or decreasing the volume is feasible but the height of the liquid in the well may affect the UV dose. This may require a re-optimization of UV time and biotin dose to achieve efficient intercalation of the psoralen.

3. Just before use, prepare the BRIGHTSTAR™ psoralen-biotin (2.3.3): dissolve the contents (4.17 ng) of the kit in 50 μL DMF (also in kit) or for EZ-LINK™ Psoralen-PEO-Biotin (2.3.4) prepare a 0.25 mg/mL solution in water.

4. Add the resuspended DNA into a UV plate for UV crosslinking. Add 1.3 μL of BRIGHTSTAR™ psoralen-biotin or 2 μL of 0.25 mg/mL EZ-LINK™ Psoralen-PEO-Biotin solution per 100 μL DNA.

5. Crosslink for 20 minutes for BRIGHTSTAR™ psoralen-biotin or for 30mins for EZ-LINK™ Psoralen-PEO-Biotin with 365 nm UV, with the plate right up to the light; plate on ice; entire set-up covered with foil. (The light covers 5 columns of the plate, so use only 5 columns of wells.) Note, 30 minutes with this set up corresponds to 8000 mJ/cm².

6. Prepare SEPHADEX™ slurry, 25-50 mg/mL in water. Add 200 μL of slurry to a 1.2 μm glass fiber filter plate. Spin briefly (1000 rpm for 1 minute, IEC Centra GP8) into a discard plate. Add 100 μL of water to the filter plate for the SEPHADEX™ to swell. Add 100 μL of DNA and spin briefly again into the collection plate. Add 100 μL water to the filter plate and spin briefly into the collection plate again.

7. Add eluate (μ250 μL) to either a MICROCON™ 100 kDa tube, or a 100 kDa 96-well filter plate stacked on top of a discard plate. For the MICROCON™ tube, spin at 1000 g for 20 minutes (Eppendorf 5417C). For the filter plate, spin for 20 minutes at 2000 rpm (IEC Centra GP8).

8. Resuspend in 50 μL water (2 μg/μL plasmid DNA). For example, DNA is prepared so that OD 260 at 1:300 dilution is approximately 0.6 (the absorbance reading is only applicable with the above mentioned method of DNA preparation; different DNA preparation methods yield different purity with different absorbance). Note: the desired final plasmid DNA concentration depends on the level of expression for the particular gene of interest. Final plasmid DNA concentration may vary from about 0.5 μg/μL for genes with high expression capacity (e.g., from 0.1 μg/μL to 0.5 μg/μL or 0.5 μg/μL to 0.8 μg/μL) to about 3 μg/μL for genes with poor expression capacity (e.g., from 1 μg/μL to 3 μg/μL or 2 μg/μL to 5 μg/μL).

9. Prepare spotting mix in arraying plate: 10 μL DNA+1.5 μL of master mix.

Master mix: For linker-only slides: GST polyclonal AB (0.5 mg/mL)+BS3 crosslinker (2 mM)+avidin (1 mg/mL) or streptavidin (3.5 mg/mL). For avidin/streptavidin coated slides: GST polyclonal AB (0.5 mg/mL)+BS3 crosslinker (2 mM).

10. GST registration spots: 0.03 mg/mL in water or PBS.

11. Mouse IgG registration spots (whole mouse IgG antibody): 0.5 mg/mL in water or PBS.

12. Spin down plate, 1 min at 1000 rpm (IEC Centra GP8).

13. Array, using humidity control at 40-60%.

14. Store spotted slides in cold room with water in the bottom of the tray, at least overnight. The bioassay dish divider should be placed in a deeper bioassay dish, so that the slides can be placed face-up on the rack without hitting the cover. Water in the bottom of the tray maintains high humidity.

15. Store slides the next day at room temperature. Storage conditions have been tested at room temperature to −80° C. in the dark for up to 2 months without loss in expression and capture.

3.4. Expression of Proteins

1. Block slides for ˜1 hr at room temperature or 4° C. overnight in the coldroom with SUPERBLOCK™ or milk. Use ˜30 mL in a pipette box for 4 slides. The slides need to be shaken during this initial step to wash away unbound NAPPA reagents (plasmid, avidin/streptavidin, capture antibody).

2. Quickly rinse with MILLI-Q™ water. Dry with filtered compressed air. Avoid letting the slides stand to dry to avoid water marks that may increase background.

3. Prepare in-vitro transcription/translation (IVT) mix. For 1 slide, 100 □L is needed: 4 μL TNT buffer; 2 μL T7 polymerase; 1 μL of -Met; 1 μL of -Leu or -Cys; 2 μL of RNaseOUT; 40 μL of DEPC water.

4. Apply a HYBRIWELL™ gasket to each slide. Use the wooden stick to rub the areas where the adhesive is to make sure it is well stuck all around.

5. Add IVT mix from the non-specimen end. Pipette the mix in slowly; it's okay if it beads up temporarily at the inlet end. Gently massage the HYBRIWELL™ to get the IVT mix to spread out and cover all of the area of the array. Apply the small round port seals to both ports.

6. Incubate for 1.5 hr at 30° C. for protein expression (30 is key; 28 or 32 gives reduced yield), followed by 30 min at 15° C. for the query protein to bind to the immobilized protein.

7. Remove the HYBRIWELL™; wash with milk 3 times, 3 minutes each, in pipette box on a shaker. Use about 30 mL per wash.

8. Block with SUPERBLOCK™ or milk overnight at 4° C. or room temperature for 1 hour.

3.5. Detection and Analysis

1. Apply primary AB (mouse anti-GST or mouse anti-HA) by adding 150 μL to the non-specimen end of the slide, then apply a coverslip. Incubate for 1 hour at room temperature; wash with milk (3 times, ˜5 min). Drain.

2. Apply secondary AB (anti-mouse HRP) by adding 150μL to the non-specimen end of the slide, then apply a coverslip. Incubate for 1 hour at room temperature; wash with PBS (3 times, ˜5 min). Then do a quick rinse with MILLI-Q™ water. Drain.

3. Before applying TSA solution, make sure slides are not too wet, but don't let them fully dry. Apply TSA mix and place coverslip. Incubate for 10 minutes at room temperature. Rinse in MILLI-Q™ water; dry with filtered compressed air.

4. Scan in microarray scanner, using settings for Cy3.

As a quality check, select a couple of slides per arraying batch, and detect the arrayed DNA:

1. Block with SUPERBLOCK™ 1 hour.

2. For a single slide: apply 150 μL PicoGreen mix, and apply coverslip. Let sit for 5 minutes at room temperature. For 4 slides, add 20 mL in a box and shake for 5 minutes.

3. Wash with PBS (3 times, ˜5 min). Then do a quick rinse with Milli-Q water.

4. Dry with filtered compressed air.

5. Scan, using Cy3 settings.

Part of the slide preparation process involves coating the slide with an activated NHS ester crosslinker (DMS). In some cases, coating of a glass slide with a crosslinker reduces background.

We have used both streptavidin and avidin to immobilize the DNA onto the array surface. We have also coated the slides with avidin or streptavidin instead of adding it to the array mixture. In some implementations, streptavidin is preferred as is including the biotin-binding reagent (e.g., avidin or streptavidin) in the mixture with the DNA prior to spotting onto the array.

In one spotting method, amounts of biotin ranged from 0.1, 0.3, 1, 3, 10, 30, 80, 250, 740, 2000, 7000, and 20 000 ng (nanograms). Amounts of plasmid DNA (e.g., about 5.5-6.5 kb in size) that can be used include 0.23, 0.69, 2.1, 6.2, 18, 55, 166, and 500 ng. Similar molar quantities of other nucleic acids and anchoring agents can also be used. Molar ratios of DNA to biotin that can be used include 1: 1, 1:3, 1:9, 1:26, and 1:77, e.g., a ratio of one to between about 0.5 to 10 or a ratio of one to between about 10 to 50.

It is often key during processing of slides to avoid allowing them to air dry. Air drying under some conditions leaves water marks which will result in high background. A clean air source can be used to quickly dry the slides. Slides can be rinsed in clean filtered water before drying especially if the arrays have been incubating in salt or protein solutions.

It is advisable to test a small sample of your prepared lysate for expression using the positive control provided in the kit.

Other embodiments are within the following claims.

1. A method of providing an array substrate, the method comprising:
disposing, on a substrate, one or more nucleic acids that comprise a coding region and an anchoring agent, the substrate comprising a plurality of addresses,
maintaining the substrate under conditions which enable the anchoring agent of each disposed nucleic acid to stably attached to the substrate, and
contacting the substrate with a transcription and/or translation effector.

2. The method of claim 1 wherein the coding region encodes a polypeptide that comprises a first amino acid sequence and a tag that can interact with a binding agent, and the method further comprising disposing, on the substrate, the binding agent.

3. The method of claim 2 wherein the binding agent and the nucleic acid are disposes contemporaneously.

4. The method of claim 1 wherein the disposing comprises disposing a solution that includes the nucleic acid attached to the anchoring agent, and the binding agent.

5. The method of claim 4 wherein the solution further includes a crosslinker.

6. The method of claim 5 wherein the solution is maintained under conditions that permit aggregates to form.

7. The method of claim 1 wherein the nucleic acid is a circular plasmid.

8. The method of claim 7 wherein the nucleic acid is supercoiled.

9. The method of claim 1 wherein the anchoring agent comprises biotin bound to a biotin binding protein.

10. The method of claim 1 wherein the substrate comprises a linker.

11. A method comprising:
providing a plurality of coding nucleic acids,
modifying each nucleic acid of the plurality to include an anchoring agent, and
disposing each nucleic acid of the plurality at an address on a substrate.

12. The method of claim 11 wherein each coding nucleic acid encodes a polypeptide that comprises a first amino acid sequence and an affinity tag.

13. The method of claim 11 wherein each address further comprises a binding agent that recognizes the affinity tag.

14. The method of claim 11 wherein each nucleic acid of the plurality is disposed at a different address.

15. The method of claim 11 wherein some nucleic acids of the plurality are disposed at the same address.

16. The method of claim 11 wherein some nucleic acids of the plurality are disposed at at least two different addresses.

17. The method of claim 11 wherein the step of providing at least one coding nucleic acid of the plurality comprises extending a source nucleic acid using a polymerase and a tagged nucleotide.

18. The method of claim 17 wherein the tagged nucleotide comprises a biotin or digoxygenin moiety.

19. A method comprising:
providing a plurality of coding nucleic acids,
stably attaching each nucleic acid of the plurality at an address on a substrate, and
translating each nucleic acid of the plurality with a translation.

20. The method of claim 19 wherein the substrate comprises positively charged groups that can interact with negative charges on nucleic acid.

21. The method of claim 19 wherein the nucleic acids of the plurality are stably attached by formation of a concatamer with a nucleic acid anchored to the surface.

22. A method of providing an array substrate:
providing a substrate that comprises a plurality of addresses, each addresses comprising (i) a binding agent and (ii) a nucleic acid that comprises (1) a coding region and (2) an anchoring agent that stably attaches the nucleic acid to the substrate, wherein the coding region encodes a polypeptide that comprises a first amino acid sequence and a tag that can interact with the binding agent, and contacting the substrate with a transcription and/or translation effector.

23. A method comprising:
contemporaneously depositing (i) a binding agent that can interact with a tag and (ii) a nucleic acid that can be stably attached to a substrate and that comprises a sequence encoding a first amino acid sequence and the tag onto a substrate.

24. The method of claim 23 wherein the step of depositing comprises providing a mixture that comprises the binding agent and the nucleic acid.

25. The method of claim 23 further comprising repeating the depositing for a plurality of nucleic acids, each being disposed at a different address on the substrate.
